(19) United States
(12) Patent Application Publication
(10) Pub. No.: US 2019/0269792 A1
(43) Pub. Date: Sep. 5, 2019

(54) NANOPARTICLE COMPOSITIONS COMPRISING CD38 AND METHODS OF USE THEREOF

(71) Applicants: Abdel Kareem Azab, St. Louis, MO (US); Maria del Pilar de la Puente Garcia, St. Louis, MO (US)

(72) Inventors: Abdel Kareem Azab, St. Louis, MO (US); Maria del Pilar de la Puente Garcia, St. Louis, MO (US)

(73) Assignee: Washington University, St. Louis, MO (US)

(21) Appl. No.: 16/345,132

(22) PCT Filed: Oct. 25, 2017
(86) PCT No.: PCT/US17/58324

§ 371 Date: Apr. 25, 2019

Related U.S. Application Data
(60) Provisional application No. 62/412,518, filed on Oct. 25, 2016.

(51) Int. Cl. A61K 47/69; A61P 35/00; C07K 16/28; A61K 9/51; A61K 47/68; A61K 31/69

(52) U.S. Cl. CPC A61K 47/6939; A61P 35/00; A61K 31/69; A61K 9/5161; A61K 47/6849; C07K 16/2896

(57) ABSTRACT

The present disclosure discloses compositions, and methods of making and using nanoparticles to treat multiple myeloma.

### CROSS REFERENCE TO RELATED APPLICATIONS

This application claims the benefit of U.S. Provisional Application No. 62/412,518, filed Oct. 25, 2016, the disclosure of which is hereby incorporated by reference in its entirety.

### FIELD OF THE INVENTION

Nanoparticles for the treatment of multiple myeloma.

### BACKGROUND OF THE INVENTION

Multiple myeloma (MM) is the second most common hematological malignancy and represents approximately 20% of deaths from hematological malignancies. Despite the introduction of novel therapies, more than 90% of MM patients relapse, due to therapy-resistant stem-cell-like MM cells and minimal residual disease (MRD). The main limiting factor for the effective use of chemotherapies in MM is the serious side effects caused by these drugs. The use of proteasome inhibitors bortezomib and carfilzomib has led to a significant improvement in the survival of MM patients. However, treatment with bortezomib is limited by its neurotoxicity, especially in the peripheral nerves, which leads to painful sensory axonal neuropathy. Carfilzomib is a second generation proteasome inhibitor, but the safety data from a meta-analysis reported thrombocytopenia, anemia, fatigue, nausea, and diarrhea as the most common adverse events, with dose-limiting neutropenia or peripheral neuropathy.

Immunomodulatory drugs are emerging promising therapies in MM which show synergistic effects when combined with current treatments. Nevertheless, one-fourth of patients discontinued immunomodulatory drugs such as thalidomide because their of their toxicity, including peripheral neuropathy, constipation, somnolence, and fatigue as common side effects. Moreover, cutaneous adverse neutropenia, deep vein thrombosis, infection, and hematologic cancer were observed in patients treated with lenalidomide. Dose limiting neutropenia, thrombocytopenia, neuropathy, and deep vein thrombosis were common adverse effects observed in patients treated with pomalidomide.

Therefore, a new strategy to specifically target MM cells which will increase the efficacy of the treatment, while reducing the side effects is needed.

### SUMMARY OF THE INVENTION

In an aspect the disclosure provides a composition for treating multiple myeloma (MM), the composition comprises chitosan nanoparticles, at least one MM cell targeting antibody conjugated to the surface of the nanoparticles, and at least one therapeutic agent encapsulated in the nanoparticles. The chitosan nanoparticles are crosslinked nanoparticles, wherein chitosan is crosslinked with sodium tripolyphosphate (TPP). The MM cell targeting antibody may be an anti-CD38 antibody, wherein the chitosan nanoparticles conjugated to the anti-CD38 antibody target MM cells expressing CD38.

In an aspect the disclosure provides a method of making therapeutic chitosan nanoparticles, the method comprising producing chitosan nanoparticles, encapsulating at least one therapeutic agent in the chitosan nanoparticles, and conjugating the chitosan nanoparticles to a targeting antibody.

In an aspect the disclosure provides a method of making anti-CD38 bortezomib (BTZ) loaded chitosan nanoparticles, the method comprising producing chitosan nanoparticles, encapsulating BTZ in the chitosan nanoparticles, and conjugating the chitosan nanoparticles with anti-CD38 antibody.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-G show the characterization of anti-CD38 chitosan nanoparticles (NPs). (FIG. 1A) Crosslinking reaction showing ionotropic gelation of chitosan with TPP anions (Chem Draw Professional 15.1 was used for the chemical drawings). (FIG. 1B) Scheme of the crosslinking of chitosan with sodium tripolyphosphate (TPP) to produce chitosan nanoparticles from soluble chitosan. (FIG. 1C) Schematic illustration of the anti-CD38-targeted chitosan nanoparticles loaded with drugs for specific targeting of MM cells. (FIG. 1D) Scheme representing anti-CD38 chitosan NPs loaded with bortezomib by conjugation of polymeric chitosan NPs with streptavidin and further coupling with biotinylated anti-CD38 monoclonal antibody. (FIG. 1E) Effect of TPP crosslinker (0.25-1 mg/ml) on size. (FIG. 1F) Effect of TPP crosslinker (0.25-1 mg/ml) on stability of anti-CD38 chitosan NPs. (FIG. 1G) The effect of TPP concentration on the z-potential of the chitosan nanoparticles.

FIG. 2A-F shows the characterization of anti-CD38 chitosan NPs. (FIG. 2A) BTZ HPLC detection peak with retention time 2 minutes, λ=270 nm. (FIG. 2B) TZ calibration curve formed by plotting the AUC of BTZ HPLC peak for the concentration range of bortezomib (0 to 1.5 mM). (FIG. 2C) Effect of intermediate (50 μM) and high-dose (1 mM) BTZ on size. (FIG. 2D) Effect of intermediate (50 μM) and high-dose (1 mM) BTZ on stability of anti-CD38 chitosan NPs. (FIG. 2E) Effect of time preservation on size. (FIG. 2F) Effect of time preservation on stability of empty or BTZ loaded anti-CD38 chitosan NPs.

FIG. 3A-E illustrate in vitro drug release from anti-CD38 chitosan NPs to different environments. (FIG. 3A) The effect of tumor environment on the release of doxorubicin from chitosan nanoparticles. (FIG. 3B) pH of non-conditioned media and conditioned media from PB MNCs and MM cells after 72 h in culture (n=3). (FIG. 3C) Effect of conditioned media pH on drug release from anti-CD38 chitosan NPs. (FIG. 3D) Corrected pH of conditioned media from PB MNCs and MM cells after 72 h in culture (n=3). (FIG. 3E) Effect of conditioned media with corrected pH on drug release from anti-CD38 chitosan NPs.

FIG. 4A-D show the in vitro drug release from anti-CD38 chitosan NPs to different environments. (FIG. 4A) pH of non-conditioned media and conditioned media from 3 independent PB MNCs patient samples and 3 MM cell lines after 72 h in culture. (FIG. 4B) Effect of conditioned media pH on drug release from anti-CD38 chitosan NPs. (FIG. 4C) Corrected pH of conditioned media from 3 independent PB MNCs and 3 MM cell lines after 72 h in culture. (FIG. 4D) Effect of conditioned media with corrected pH on drug release from anti-CD38 chitosan NPs.

FIG. 5A-H show the kinetics of binding of anti-CD38 chitosan NPs to MM cells. FIG. 5A-C show the representative histograms of CD38 expression measured as fold of MFI of anti-CD38 to isotypes controls in MM.1S (FIG. 5A), H929 (FIG. 5B), and RPMI (FIG. 5C) MM cell lines. (FIG. 5D) Correlation of anti-CD38 NPs binding at 2 h with CD38 expression in MM cells. (FIG. 5E) Kinetics of binding of anti-CD38 chitosan NPs over a period of time of 24 h to MM cells. (FIG. 5F-H) Fluorescent imaging of AF633 anti-CD38 chitosan NPs after 2 h of binding to MM1s cells shown in Bright field (BF) (FIG. 5F); AF633, Red (FIG. 5G) and merged (FIG. 5H). Scale bar=10 μm. (FIG. 5I) Kinetics of dissociation of anti-CD38 chitosan NPs over a period of time of 24 h to MM cells after 2 h of binding.

FIG. 6A-D show specificity and variability of binding of anti-CD38 chitosan NPs. Binging of anti-CD38 chitosan NPs, non-targeted chitosan NPs and unstained control NPs to (FIG. 6A) MM cell lines (MM1S, H929, OPM1, RPMI, U266), (FIG. 6B) CD138+ cells isolated from MM patients (n=5), (FIG. 6C) mononuclear cells isolated from the peripheral blood of normal subjects (PB MNCs, n=5), (FIG. 6D) and normal plasma cells isolated from the BM of normal subjects (BM MNCs, n=5) analyzed by flow cytometry.

FIG. 7A-E show the specificity of binding of anti-CD-38 chitosan NPs to MM cells. (FIG. 7A) The effect of hypoxia, in vitro, on the expression of plasma and B-cell markers. (FIG. 7B) The specificity of the binding of non-targeted and anti-Cd38-targeted chitosan nanoparticles to MM1s cells and normal mononuclear cells. (FIG. 7C) Comparison of specificity of binging of anti-CD38 chitosan NPs versus non-targeted chitosan NPs to MM cell lines (Av, n=5), CD138+ cells isolated from MM patients (Av, n=5), normal mononuclear cells isolated from the peripheral blood of normal subjects (PB MNCs, Av, n=5) and normal plasma cells isolated from the BM of normal subjects (BM MNCs, Av, n=5) analyzed by flow cytometry, *p<0.05. (FIG. 7D) Mechanism of binding of the anti-CD38 targeted and non-targeted NPs by blocking or not with free anti-CD38 antibody, *p<0.05. (FIG. 7E) Comparison of the biodistribution of anti-CD38 chitosan NPs, non-targeted chitosan NPs and free deactivated AF633 to MM cells (GFP+) in different organs: femurs, blood, heart, kidney, liver, lung and spleen, analyzed by fluorescent signal (fold of MFI of AF633) by flow cytometry, *p<0.05. (n=5).

FIG. 8A-B show the effect of bortezomib-loaded anti-CD38 chitosan NPs on proliferation, cell cycle, and apoptosis. The effect of vehicle (control), BTZ as a free drug (5 nM), empty chitosan NPs, non-targeted chitosan NPs loaded with BTZ equivalent amounts to 5 nM, and anti-CD38 chitosan NPs loaded with BTZ equivalent amounts to 5 nM for 48 h on: (FIG. 8A) proliferation of MM1s, H929, RPMI cells and PB MNCs analyzed by MTT, *p<0.05. (FIG. 8B) Cell cycle of H929 cells measured by Propidium Iodide stained of DNA, *p<0.05.

FIG. 9A-D show the effect of bortezomib-loaded anti-CD38 chitosan NPs on proliferation, cell cycle, and apoptosis. The effect of vehicle (control), BTZ as a free drug (5 nM), empty chitosan NPs, non-targeted chitosan NPs loaded with BTZ equivalent amounts to 5 nM, and anti-CD38 chitosan NPs loaded with BTZ equivalent amounts to 5 nM for 48 h on: (FIG. 9A) proliferation of MM cells and PB MNCs analyzed by MTT, *p<0.05; (FIG. 9B) % Sub-G1 population of H929 cells measured by Propidium Iodide stained of DNA, *p<0.05; (FIG. 9C) Apoptosis of H929 cells by FITC-Annexin-V and Propidium Iodide, *p<0.05. (FIG. 9D) Survival-associated molecules (pERK and pAKT) after 6 h of treatment, cell cycle-associated molecule (pRB), as well as, apoptotic-associated molecules (cleaved PARP and cleaved caspase 3) after for 12 h of treatment, in H929 cells were measured by immunoblotting.

FIG. 10A-B show the enhanced bortezomib uptake and proteasome inhibition by anti-CD38 chitosan NPs. (FIG. 10A) Cell pellet boron concentration analyzed by ICP-OES after no treatment (control), BTZ as a free drug (100 nM), and anti-CD38 chitosan NPs loaded with BTZ equivalent amounts to 100 nM for 1.5 h, *p<0.05. (FIG. 10B) The effect of no treatment (control), BTZ as a free drug (5 nM), empty chitosan NPs, non-targeted chitosan NPs loaded with BTZ equivalent amounts to 5 nM, and anti-CD38 chitosan NPs loaded with BTZ equivalent amounts to 5 nM for 2 h on proteasome activity, *p<0.05.

FIG. 11A-D shows the effect of macropinocytosis and endocytosis inhibitors on survival of MM cells. (FIG. 11A) Incubation with macropinocytosis inhibitor Cytochalasin D (0-1.5 μM) for 3 0 min. (FIG. 11B) Incubation with early endocytosis chlathrin-mediated inhibitor Chlorpromazine (0-3 μM) for 30 min. (FIG. 11C) Incubation with early endocytosis caveolae-mediated inhibitor Nystatin (0-1 μg/ml) for 30 min. (FIG. 11D) Incubation with late endocytosis inhibitor EGA (0-5 μM) for 30 min.

FIG. 12A-I show the enhanced proteasome activity inhibition by anti-CD38 chitosan NPs endocytic internalization. FIG. 12A-F show confocal micrographs of the MM cells showing intracellular location of AF633 anti-CD38 in red, early endosomes expressing GFP (rab5+), and merged images showing co-localization of the red NPs in the green early endosomes at 6 and 24 hours; Scale bar=10 μm. (FIG. 12A) Early endosomes expressing GFP (rab5+) at 6 hours, (FIG. 12B) intracellular location of AF633 anti-CD38 in red at 6 hours, and (FIG. 12C) merged images showing co-localization of the red NPs in the green early endosomes at 6 hours. (FIG. 12D) Early endosomes expressing GFP (rab5+) at 6 hours, (FIG. 12E) intracellular location of AF633 anti-CD38 in red at 6 hours, and (FIG. 12F) merged images showing co-localization of the red NPs in the green early endosomes at 24 hours. (FIG. 12G) Anti-CD38 chitosan NPs uptake in the presence of macropinocitosys and endocytosis inhibitors. H929 cells were pre-incubated with the following inhibitors cytochalasin D, chlorpromazine, nystatin, and EGA for 30 min. Two control samples were used with no inhibitors (No inhibition) at either 37° C. or 4° C., *p<0.05. (FIG. 12H) The effect of anti-CD38 chitosan NPs loaded with BTZ equivalent amounts to 5 nM uptake in the presence of macropinocitosys and endocytosis inhibitors on proteasome activity, *p<0.05. (FIG. 12I) The effect of BTZ as a free drug (5 nM) in the presence of macropinocitosys and endocytosis inhibitors on proteasome activity, *p<0.05.

FIG. 13A-G show the effect of bortezomib-loaded anti-CD38 chitosan NPs on drug resistance. (FIG. 13A-B) The effect of vehicle (control), BTZ as a free drug (5 nM), empty chitosan NPs, non-targeted chitosan NPs loaded with BTZ equivalent amounts to 5 nM, and anti-CD38 chitosan NPs loaded with BTZ equivalent amounts to 5 nM for 48 h on: (FIG. 13A) cell adhesion-mediated drug resistance in co-culture with MSP-1 (myeloma-derived stroma); and (FIG. 13B) hypoxia-mediated drug resistance, *p<0.05. (FIG. 13C) Effect of vehicle (control), BTZ as a free drug (10 nM), empty chitosan NPs, non-targeted chitosan NPs loaded with BTZ equivalent amounts to 10 nM, and anti-CD38 chitosan NPs loaded with BTZ equivalent amounts to 10 nM for 48 h on survival of MM cells cultured in 3DTEBM, *p<0.05. (FIG. 13D) shows confocal microscopy images of MM cells cultured (green) in 3DTEBM after 24 h treatment with AF633 anti-CD38 chitosan NPs (red), shown by a Z-Stack rotated view, and images at different depths of the z-stack to show the co-localization (white arrows) of NPs and MM cells in yellow; (FIG. 13E) Top Frame_5, (FIG. 13F) Middle Frame_48, and (FIG. 13G) Bottom frame_90; Scale bar=50 μm.

FIG. 14A-H shows the inhibition of of tumor progression and reduction of the side effects of bortezomib-loaded anti-CD38 chitosan NPs in vivo. The effect of vehicle, BTZ as a free drug (1 mg/kg once a week), non-targeted chitosan NPs loaded with BTZ equivalent amounts to 1 mg/kg (once a week), and anti-CD38 chitosan NPs loaded with BTZ equivalent amounts to 1 mg/kg (once a week) on: (FIG. 14A) shows tumor progression shown by quantification of BLI*p<0.05; (FIG. 14B) shows number of MM cells on circulation, *p<0.05; FIG. 14C shows survival shown by Kaplan-Meier survival curves (p value targeted compared to other treatments); FIG. 14D shows % weight loss, and FIG. 14E-H shows hair loss with representative images of one mice from each group, (FIG. 14E) vehicle, (FIG. 14F) BTZ (1 mg/kg), (FIG. 14G) BTZ non-targeted NPs, and (FIG. 14H) BTZ anti-CD38 NPs.

FIG. 15A-F shows an evaluation for histological tissue damage. The effect of vehicle, BTZ as a free drug (1 mg/kg once a week), non-targeted chitosan NPs loaded with BTZ equivalent amounts to 1 mg/kg (once a week), and anti-CD38 chitosan NPs loaded with BTZ equivalent amounts to 1 mg/kg (once a week) on histological tissue damage on femur, spinal cord, liver, spleen, kidney, intestine after 25 days of treatment. (FIG. 15A) Histological image of femur of vehicle, BTZ as a free drug, non-targeted chitosan NPs loaded with BTZ, and anti-CD38 chitosan NPs loaded with BTZ group. (FIG. 15B) Histological image of spinal cord of vehicle, BTZ as a free drug, non-targeted chitosan NPs loaded with BTZ, and anti-CD38 chitosan NPs loaded with BTZ group. (FIG. 15C) Histological image of liver of vehicle, BTZ as a free drug, non-targeted chitosan NPs loaded with BTZ, and anti-CD38 chitosan NPs loaded with BTZ group. (FIG. 15D) Histological image of spleen of vehicle, BTZ as a free drug, non-targeted chitosan NPs loaded with BTZ, and anti-CD38 chitosan NPs loaded with BTZ group. (FIG. 15E) Histological image of kidney of vehicle, BTZ as a free drug,. non-targeted chitosan NPs loaded with BTZ, and anti-CD38 chitosan NPs loaded with BTZ group. (FIG. 15F) Histological image of intestine of vehicle, BTZ as a free drug, non-targeted chitosan NPs loaded with BTZ, and anti-CD38 chitosan NPs loaded with BTZ group.

FIG. 16A-B are schematic representations of BTZ cellular uptake by normal and MM cells. (FIG. 16A) Free BTZ penetrates into all cells by passive diffusion and preferentially inhibits the overexpressed proteasome in MM cells, even at a low drug concentration. (FIG. 16B) BTZ-loaded anti-CD38 chitosan NPs entered CD38+ MM cells, at least in part, via the endocytic pathway (inhibited by 4° C. incubation). Clathrin- (inhibited by chlorpromazine, chlorp.), caveolae-mediated endocytosis (inhibited by nystatin) incorporated anti-CD38 chitosan NPs into early endosomes, which will be transformed into late endosomes (inhibited by EGA). From the late endosomes, BTZ is presented at a higher drug concentration to the proteasome.

### DETAILED DESCRIPTION OF THE INVENTION

The present disclosure relates to compositions of a nanoparticle delivery system, methods of making a nanoparticle delivery system, and methods of using a nanoparticle delivery system to deliver active agents specifically to target cells. A nanoparticle delivery system of the disclosure may comprise crosslinked chitosan nanoparticles that encapsulate an active agent that is specifically delivered to cells. The active agent may be a therapeutic agent or a diagnostic agent. The surface of the chitosan nanoparticles is conjugated to specific antibodies that recognize and bind to antigens on the surface of target cells. Nanoparticles that are bound specifically to the target cells may be taken into the cells by an active process such as endocytosis. Compositions and methods of the nanoparticle delivery system are described below.

### Composition

The present disclosure provides for a nanoparticle delivery system. The composition comprises nanoparticles that have at least one cell targeting molecule on the surface of the nanoparticles and at least one active agent encapsulated in the nanoparticles. A composition of the present disclosure may also comprise a suitable pharmaceutically acceptable carrier known in the art.

As used herein, the term nanoparticle refers to a particle that has a diameter of less than 1 um (1000 nm). Nanoparticles may be substantially spherical in shape and the diameter of a group of nanoparticles may be represented by the average diameter of the nanoparticles in the group.

As used herein “cell targeting” refers to a property of the nanoparticles of the disclosure, to home to and bind specific cells of interest that may express a specific molecule, such as an antigen, to which a targeting molecule associated with a nanoparticle binds.

As used herein, “target” refers to the cell of interest to which a targeting molecule of a nanoparticle of the present disclosure binds. The term “target” also encompasses a cell of interest to which delivery of an active agent is desired.

In an aspect a nanoparticle of the disclosure may have a size that ranges from about 20 nm to about 100 nm. In various aspects, a nanoparticle of the present disclosure may have a diameter from about 15 nm to about 30 nm, from about 25 nm to about 40 nm, from about 35 nm to about 55 nm, from about 45 nm to about 75 nm, from about 70 nm to about 95 nm, from about 90 nm to about 115 nm, from about 105 nm to about 125 nm. In a preferred aspect, the size of a nanoparticle of the present disclosure is from about 35 nm to about 65 nm. In some aspects, the size of a nanoparticle of the present disclosure may be about 50±11 nm.

The effective surface electric charge, the zeta potential (ζ-potential) of a nanoparticle, plays a role in the stability of a nanoparticle. A higher ζ-potential leads to a higher stability of the nanoparticle. In an aspect, a nanoparticle of the disclosure may have a ζ-potential that ranges from about 30 mV to about 54 mV. In various aspects, a nanoparticle of the present disclosure may have a ζ-potential from about 15 mV to about 25 mV, from about 20 mV to about 35 mV, from about 25 mV to about 50 mV, from about 35 MV to about 60 mV, or from about 45 mV to about 75 mV. In a preferred aspect, the ζ-potential of a nanoparticle of the present disclosure is from about 50 mV to about 55 mV. In some embodiments, the ζ-potential of a nanoparticle of the present disclosure is about 53 mV.

Nanoparticles of the present disclosure may be constructed by a variety of materials. Non-limiting examples of the materials a nanoparticle may be constructed from may include polymers, lipids, inorganic substances, and biological materials. In an aspect, a nanoparticle of the present disclosure may be constructed of a polysaccharide such as a chitosan. In a preferred aspect, a nanoparticle of the disclosure is a chitosan-tripolyphosphate nanoparticle, e.g. chitosan molecules crosslinked with sodium tripolyphosphate(TPP) molecules.

Chitosan is a natural, non-toxic, biodegradable polysaccharide. In solution the free amino groups on its polymeric chains can protonate, giving it a positive charge. Chitosan nanoparticles may be formed by incorporating or crosslinking a polyanion, such as TPP, into a chitosan solution under constant stirring. Chitosan has poor solubility at a pH above 6.5; therefore chitosan nanoparticles are stable at higher pH levels. In contrast, chitosan is soluble in acidic conditions, so at lower pH levels, chitosan nanoparticles of the present invention may disintegrate. The solubility of chitosan particles in acidic conditions may be used to release therapeutic agents that are carried by chitosan nanoparticles of the present invention specifically in acidic conditions. For instance, tumor cells that produce acidic metabolites are generally known to develop acidic microenvironments. For example, a chitosan nanoparticle particle encapsulating a therapeutic agent may specifically release the encapsulated therapeutic agent in an acidic microenvironment of tumor cells.

In an aspect, a nanoparticle delivery system of the present disclosure may be used to deliver an active agent to a cell or site of interest. In an aspect, a nanoparticle of the disclosure may encapsulate an active agent. As used herein “active agents” may be therapeutic agents, diagnostic agents, or a combination thereof. Non-limiting examples of an active agent may include proteasome inhibitors, histone deacetylase inhibitors, chemotherapeutic agents, immunomodulating agents, or other agents that may be toxic to or kill cancer cells. Non-limiting examples of proteasome inhibitors may include bortezomib, carfilzomib, marizomib, ixazomib, or MLN9708. Non-limiting examples of a histone deacetylase inhibitor may be panobinostat, vorinostat, zolinza, romidepsin, or Istodax. Non limiting examples of chemotherapeutic agents may be doxorubicin, melphalan, vincristine, cyclophosphamide, etoposide, or bendamustine. Non-limiting examples of immunomodulating agents may be thalidomide, lenalidomide, or pomalidomide.

In an aspect, the active agent encapsulated in a nanoparticle of the present invention may be bortezomib. In another aspect, the active agent encapsulated in a nanoparticle of the present invention may be doxorubicin. In yet another aspect, an active agent encapsulated in a nanoparticle may be a combination of bortezomib and doxorubicin.

A nanoparticle of the present disclosure may release an active agent inside a cell of interest or at a site of interest. In an aspect, a nanoparticle may have controlled release properties, that is, be able to release an active agent inside a cell of interest or at a site of interest over a period of time. In some aspects, disclosed nanoparticles may substantially immediately release the active agent, in the cell or site of interest. The release of an active agent from a nanoparticle depends, in part, on the pH of the environment of the nanoparticle.

As used herein, the term “targeting molecule” refers to a molecule that may bind to a specific molecule on a target, and that directs a nanoparticle that comprises an active agent to a particular location or cell. . . . A targeting molecule may be attached to the surface of a nanoparticle through covalent, non-covalent, or other associations. . . . Non-limiting examples of targeting molecules may include synthetic compounds, natural compounds or products, macromolecular entities, and bioengineered molecules, and may include antibodies, antibody fragments, polypeptides, lipids, polynucleotides, and small molecules such as neurotransmitters, ligands, and hormones.

In an aspect, the targeting molecule may be an antibody. As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring).

In an alternate aspect of the disclosure, in addition to a targeting molecule, a nanoparticle may be attached to other molecules that may facilitate or enhance the therapeutic efficiency, efficient of delivery and uptake by cells of interest.

In an aspect, the targeting molecule specifically targets an antigen expressed on the “target,” for instance, a cell of interest. Non-limiting examples of suitable targets may include cells of MM, acute myeloid leukemia, non-Hodgkin's lymphoma, chronic lymphocytic leukemia, colorectal cancer, or non-small-cell lung cancer. Non-limiting examples of additional targets may include CD38, CD56, CD138, CD20, CD52, CD33, CD20, epidermal growth factor receptor, epithelial cell adhesion molecule, human epidermal growth factor receptor, lewis antigen, or carcinoembryonic antigen. In a preferred aspect, nanoparticles of the present disclosure may target cells expressing CD38, such as MM cells.

A nanoparticle of the disclosure may carry an active agent. The active agent, which may be a therapeutic agent may be associated with the surface of, encapsulated within, surrounded by, or dispersed throughout the nanoparticle. In a preferred aspect of the disclosure, an active agent is encapsulated within the core of a nanoparticle.

A pharmaceutical composition of the invention may also comprise one or more nontoxic pharmaceutically acceptable carriers, adjuvants, excipients, and vehicles as desired. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with a nanoparticle of the invention, use thereof in the compositions is contemplated. Supplementary active compounds may also be incorporated into the compositions.

A pharmaceutical composition of the invention may be formulated to be compatible with its intended route of administration. Suitable routes of administration may include parenteral, oral, pulmonary, transdermal, transmucosal, and rectal administration. The term parenteral, as used herein, includes subcutaneous, intravenous, intramuscular, intrathecal, or intrasternal injection, or infusion techniques.

Solutions or suspensions used for parenteral, intradermal, or subcutaneous application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH may be adjusted with acids or bases, such as hydrochloric acid or sodium hydroxide. The parenteral preparation may be enclosed in ampoules, disposable syringes or multiple dose vials made of glass or plastic.

Oral compositions generally may include an inert diluent or an edible carrier. Oral compositions may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents and/or adjuvant materials may be included as part of the composition. The tablets, pills, capsules, troches, and the like, may contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressured container or dispenser which contains a suitable propellant, e.g., a gas such as carbon dioxide, or a nebulizer.

In preferred embodiments, a pharmaceutical composition of the invention is formulated to be compatible with parenteral administration. For instance, pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.), or phosphate buffered saline (PBS). In exemplary embodiments, a pharmaceutical composition of the invention is formulated with phosphate buffered saline (PBS).

In all cases, a composition may be sterile and may be fluid to the extent that easy syringeability exists. A composition may be stable under the conditions of manufacture and storage, and may be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it may be preferable to include isotonic agents, for example, sugars, polyalcohols such as mannitol, sorbitol, or sodium chloride, in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Systemic administration may also be by transmucosal or transdermal means. For transmucosal or transdermal administration, penetrants appropriate to the barrier to be permeated are used in the formulation. Such penetrants are generally known in the art, and may include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. The compounds may also be prepared in the form of suppositories (e.g., with conventional suppository bases such as cocoa butter and other glycerides) or retention enemas for rectal delivery.

In one embodiment, a nanoparticle of the present invention may be prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art.

Additional formulations of pharmaceutical nanoparticle compositions may be in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. A suitable pharmaceutically acceptable carrier to maintain optimum stability, shelf-life, efficacy, and function of the nanoparticles would be apparent to one of ordinary skill in the art.

### Methods of Making Nanoparticles

In an aspect, nanoparticles of the disclosure may be prepared by crosslinking chitosan molecule with lower molecular weight molecules, such as TPP. In an aspect, chitosan units may be crosslinked with TPP units by an ionic interaction between the positively charged amino groups of chitosan and the negatively charged phosphate groups of TPP resulting in the chitosan polymeric nanoparticles. In some embodiments, other crosslinking compounds with negative charged groups may be used instead of TPP, or in addition to TPP.

In an aspect, chitosan nanoparticles may be obtained by the ionic crosslinking of chitosan with TPP, using about 1 mg/ml to 5 mg/ml chitosan solution and about 0.25 mg/ml to about 1 mg/ml of TPP. In various aspects, chitosan nanoparticles may be obtained by the ionic crosslinking of chitosan with TPP, using from about 0.5 mg/ml to about 1.5 mg/ml chitosan solution, from about 1 mg/ml to about 2.5 mg/ml chitosan solution, or from about 1.5 mg/ml to about 3 mg/ml chitosan solution. In various aspects, chitosan nanoparticles may be obtained by the ionic crosslinking of chitosan with TPP, using from about 0.15 mg/ml to about 0.3 mg/ml TPP solution, from about 0.25 mg/ml to about 0.5 mg/ml TPP solution, from about 0.4 to about 0.8 mg/ml TPP solution, or from about 0.75 mg/ml to about 1.25 mg/ml TPP solution,

In a preferred aspect, chitosan nanoparticles of the disclosure are prepared by ionic crosslinking of an about 2 mg/ml chitosan solution dissolved with a TPP solution (about 0.25-about 1 mg/ml). In a preferred aspect, chitosan nanoparticles of the disclosure are prepared by ionic crosslinking of an about 2 mg/ml chitosan solution dissolved with a TPP solution of about 0.25 mg/ml.

In an aspect, chitosan nanoparticles of the present disclosure are prepared by adding a chitsan solution dropwise into a TPP solution, to reach a chitosan to TPP ratio of from approximately 5:1 to approximately 4:1. In various aspects of the disclosure the ratio of chitosan to TPP ranges from about 7:1 to about 5:1, from about 6:1 to about 4:1, and from about 5:1 to about 3:1. In a preferred aspect of the disclosure, the ratio of chitosan to TPP is approximately 5:1.

The nanoparticle may be associated with an active agent at the surface of, encapsulated within, surrounded by, or dispersed throughout the nanoparticle. In an aspect, an active agent is encapsulated within the core of the nanoparticle. In an aspect, an active agent may be encapsulated before crosslinking chitosan and TPP, by adding an active agent into the crosslinking TPP solution, before the addition of a chitosan solution. In an alternate aspect, the active agent may be encapsulated after crosslinking by incubating nanoparticles in a solution of active agent.

In an aspect, nanoparticles may encapsulate active agents, using a concentration of active agent ranging from about 50 uM to about 1 mM. In an aspect, nanoparticles may encapsulate BTZ at the concentration of about 50 uM to about 1 mM. In various aspects, concentrations of BTZ ranging from about 25 uM to about 250 uM, from about 200 uM to about 500 uM, from about 250 μm to about 750 uM, or from about 700 uM to about 1.25 mM, may be used.

In an aspect, a targeting molecule that binds to a molecule expressed on a cell of interest may be attached to the surface of a nanoparticle of the disclosure. In an aspect, a nanoparticle may have at least one targeting molecule linked to its surface. A targeting molecule may be linked covalently, noncovalently, or coordinately to the surface of the nanoparticle. A targeting molecule may be linked directly or indirectly to a nanoparticle surface. For example, a targeting molecule may be linked directly to the surface of a nanoparticle or indirectly through an intervening linker. Methods of directly or indirectly linking a targeting molecule to a nanoparticles, linker design and linker synthesis are well known in the art.

In an aspect, a targeting molecule may be an antibody conjugated to a nanoparticle of the disclosure. As used herein, the term conjugated when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated. In a preferred aspect, a targeting molecule is conjugated to a nanoparticle of the disclosure by a streptavidin-biotin conjugation.

### Method of Administration the Nanoparticle Delivery System

In an aspect, the present invention encompasses administering a therapeutically effective amount of a nanoparticle composition to a subject in need thereof. As used herein, the phrase “a subject in need thereof” refers to a subject in need of preventative or therapeutic treatment. A subject may be a rodent, a human, a livestock animal, a companion animal, or a zoological animal. In one embodiment, a subject may be a rodent, e.g., a mouse, a rat, a guinea pig, etc. In another embodiment, a subject may be a livestock animal. Non-limiting examples of suitable livestock animals may include pigs, cows, horses, goats, sheep, llamas and alpacas. In still another embodiment, a subject may be a companion animal. Non-limiting examples of companion animals may include pets such as dogs, cats, rabbits, and birds. In yet another embodiment, a subject may be a zoological animal. As used herein, a “zoological animal” refers to an animal that may be found in a zoo. Such animals may include non-human primates, large cats, wolves, and bears. In a preferred embodiment, a subject is a mouse. In another preferred embodiment, a subject is a human.

A nanoparticle composition of the invention is formulated to be compatible with its intended route of administration. Suitable routes of administration include parenteral, oral, pulmonary, transdermal, transmucosal, and rectal administration. In preferred embodiments, a pharmaceutical composition of the invention is administered by injection.

One of skill in the art will recognize that the amount and concentration of the composition administered to a subject will depend in part on the subject and the reason for the administration. Methods for determining optimal amounts are known in the art.

Compositions of the invention are typically administered to a subject in need thereof in an amount sufficient to provide a benefit to the subject. This amount is defined as a “therapeutically effective amount.” A therapeutically effective amount may be determined by the efficacy or potency of the particular composition, the disorder being treated, the duration or frequency of administration, the method of administration, and the size and condition of the subject, including that subject's particular treatment response. A therapeutically effective amount may be determined using methods known in the art, and may be determined experimentally, derived from therapeutically effective amounts determined in model animals such as the mouse, or a combination thereof. Additionally, the route of administration may be considered when determining the therapeutically effective amount. In determining therapeutically effective amounts, one skilled in the art may also consider the existence, nature, and extent of any adverse effects that accompany the administration of a particular compound in a particular subject.

### Method of Treatment

In preferred aspects, a method of the invention is used to treat a neoplasm or cancer. The neoplasm may be malignant or benign, the cancer may be primary or metastatic; the neoplasm or cancer may be early stage or late stage. A cancer or a neoplasm may be treated by delivering nanoparticles carrying a therapeutic agent to at least one cancer cell in a subject. The cancer or neoplasm may be treated by slowing cancer cell growth or killing cancer cells.

In some aspects, the nanoparticle delivery system of the disclosure may treat a cancer or a neoplasm by delivering a therapeutic nanoparticle to a cancer cell in a subject in vivo. Non-limiting examples of neoplasms or cancers that may be treated with a method of the invention may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-cell lymphoma (cutaneous), T-cell leukemia and lymphoma, testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, or Wilms tumor (childhood). In a preferred embodiment, a method of the invention may be used to treat T-cell leukemia and lymphoma. In an exemplary embodiment, a method of the disclosure is used to treat MM in a subject.

In other aspects, a nanoparticle delivery system of the disclosure may deliver a therapeutic nanoparticle to a cancer cell in vitro. A cancer cell may be a cancer cell line cultured in vitro. In some alternatives of the embodiments, a cancer cell line may be a primary cell line that is not yet described. Methods of preparing a primary cancer cell line utilize standard techniques known to individuals skilled in the art. In other alternatives, a cancer cell line may be an established cancer cell line. A cancer cell line may be adherent or non-adherent, or a cell line may be grown under conditions that encourage adherent, non-adherent or organotypic growth using standard techniques known to individuals skilled in the art. A cancer cell line may be contact inhibited or non-contact inhibited.

In some embodiments, the cancer cell line may be an established human cell line derived from a tumor. Non-limiting examples of cancer cell lines derived from a tumor may include the MM cell lines MM.1S, H929, and RPMI, osteosarcoma cell lines 143B, CAL-72, G-292, HOS, KHOS, MG-63, Saos-2, or U-2 OS; the prostate cancer cell lines DU145, PC3 or Lncap; the breast cancer cell lines MCF-7, MDA-MB-438 or T47D; the myeloid leukemia cell line THP-1, the glioblastoma cell line U87; the neuroblastoma cell line SHSY5Y; the bone cancer cell line Saos-2; the colon cancer cell lines WiDr, COLO 320DM, HT29, DLD-1, COLO 205, COLO 201, HCT-15, SW620, LoVo, SW403, SW403, SW1116, SW1463, SW837, SW948, SW1417, GPC-16, HCT-8, HCT 116, NCI-H716, NCI-H747, NCI-H508, NCI-H498, COLO 320HSR, SNU-C2A, LS 180, LS 174T, MOLT-4, LS513, LS1034, LS411N, Hs 675.T, CO 88BV59-1, Co88BV59H21-2, Co88BV59H21-2V67-66, 1116-NS-19-9, TA 99, AS 33, TS 106, Caco-2, HT-29, SK-CO-1, SNU-C2B or SW480; B16-F10, RAW264.7, the F8 cell line, or the pancreatic carcinoma cell line Panc1. In an exemplary embodiment, a method of the disclosure may be used to contact a cell of a MM cell line.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Experiment 1. Characterization of Anti-CD38 Chitosan NPs.

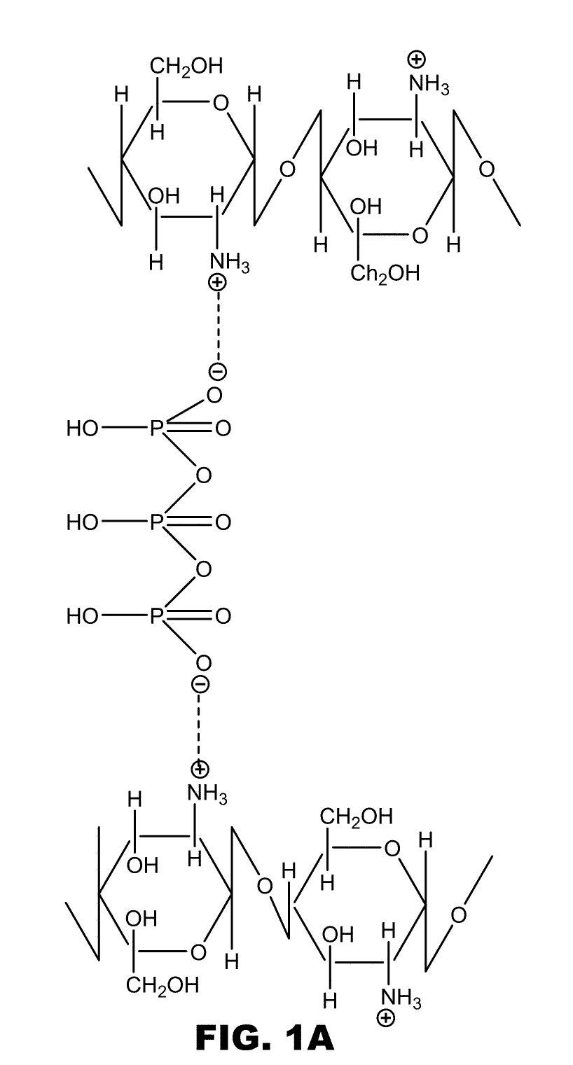

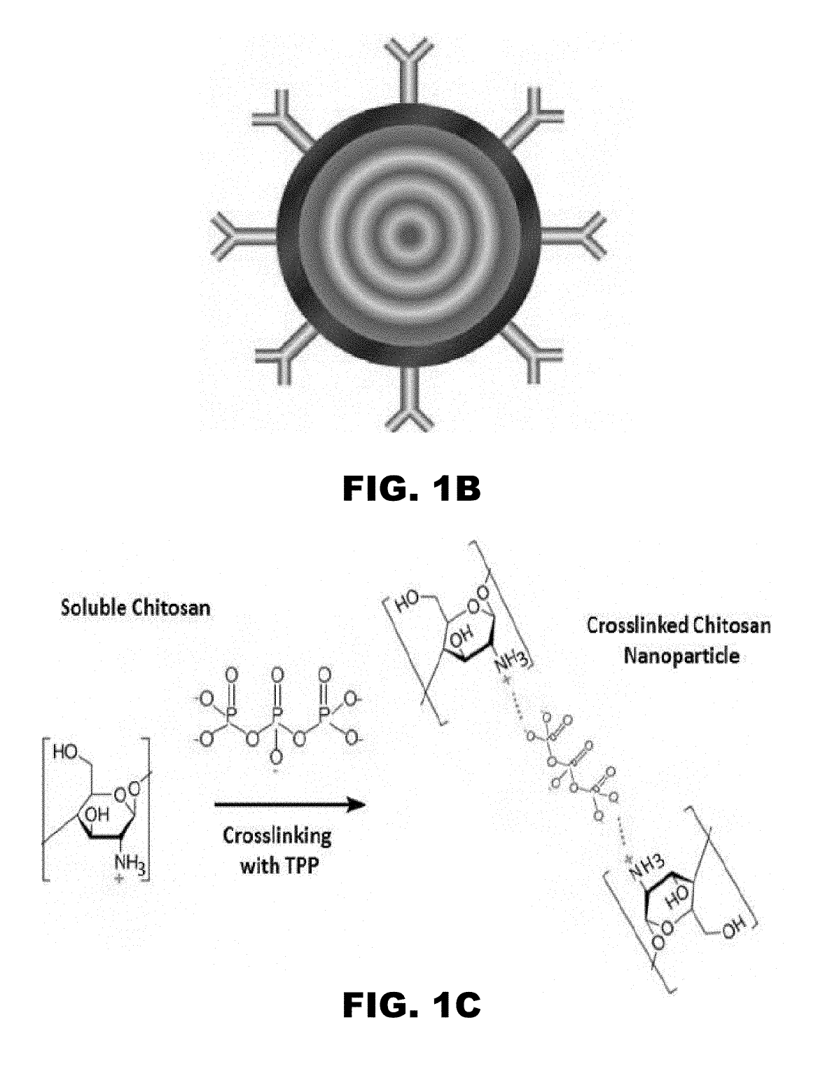

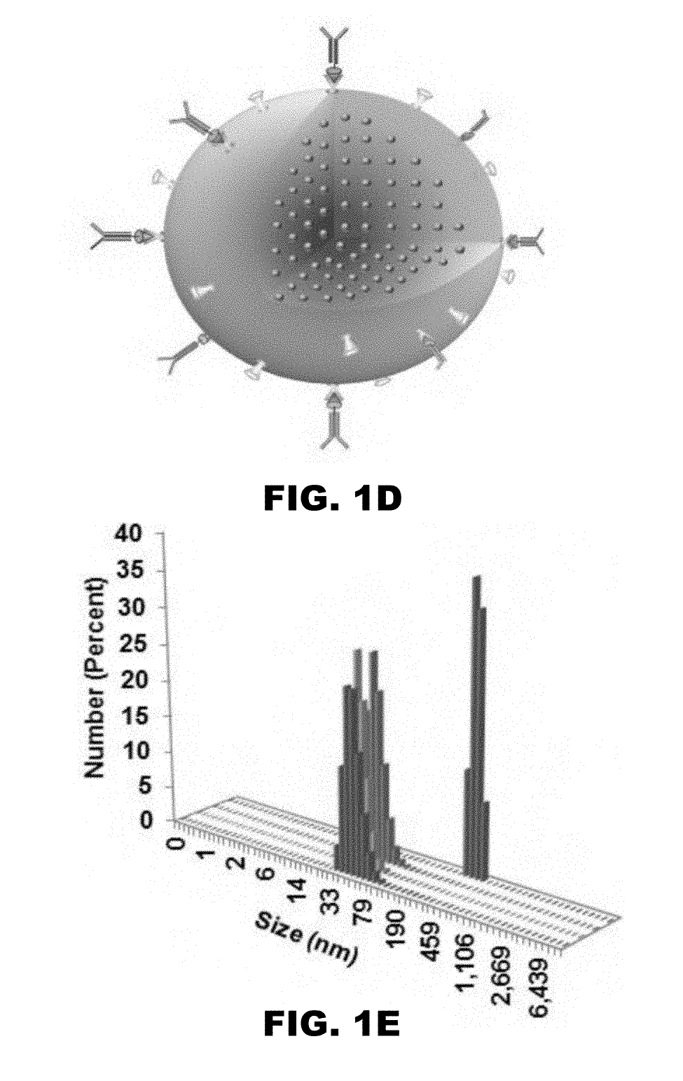

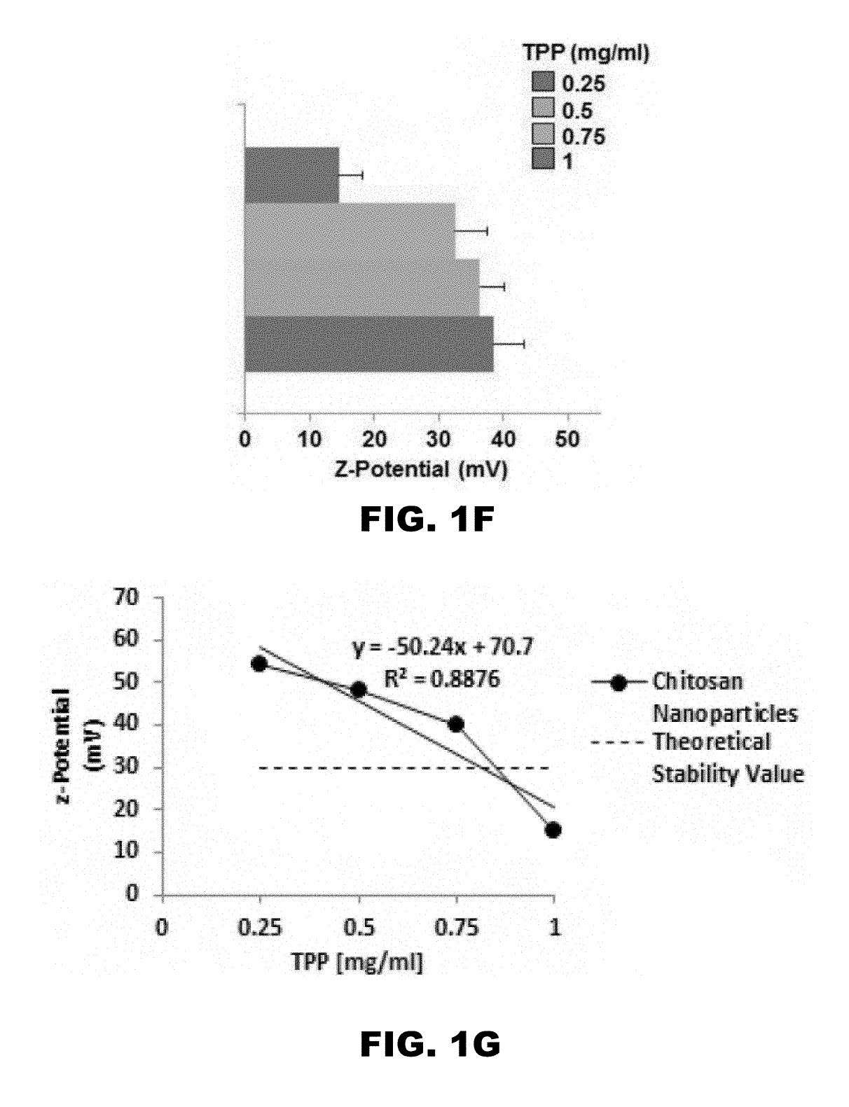

Chitosan NPs were prepared using ionotropic gelation technique in which the crosslinking reaction involves ionic interactions between the positively charged amino groups of chitosan and the negatively charged phosphate groups of TPP resulting in polymeric NPs (FIG. 1A). Targeting with anti-CD38 antibody was obtained by conjugation of chitosan NPs with streptavidin and followed by incubation with biotinylated-anti-CD38 antibody (FIG. 1D). We investigated the effect of the concentration of the crosslinker (TPP) on the size and the stability of the resulting NPs. The particle size and ζ-potential were determined by DLS. We found that increasing the concentration of the crosslinker increased the size of the particle (FIG. 1E) and it decreased the stability of the particles (FIG. 1F). A concentration of 0.25 mg/ml led to the highest ζ-potential, while 1 mg/ml reduced it dramatically to (15.1 mV) a level less than the theoretical value needed to produce stable particles (30 mV). These results showed that the crosslinking using 0.25 mg/ml maintained the highest stability of the nanoparticle with ζ-potential of 53.3 mV and the size of the particles obtained was 50±11 nm. We used these conditions for all the further studies.

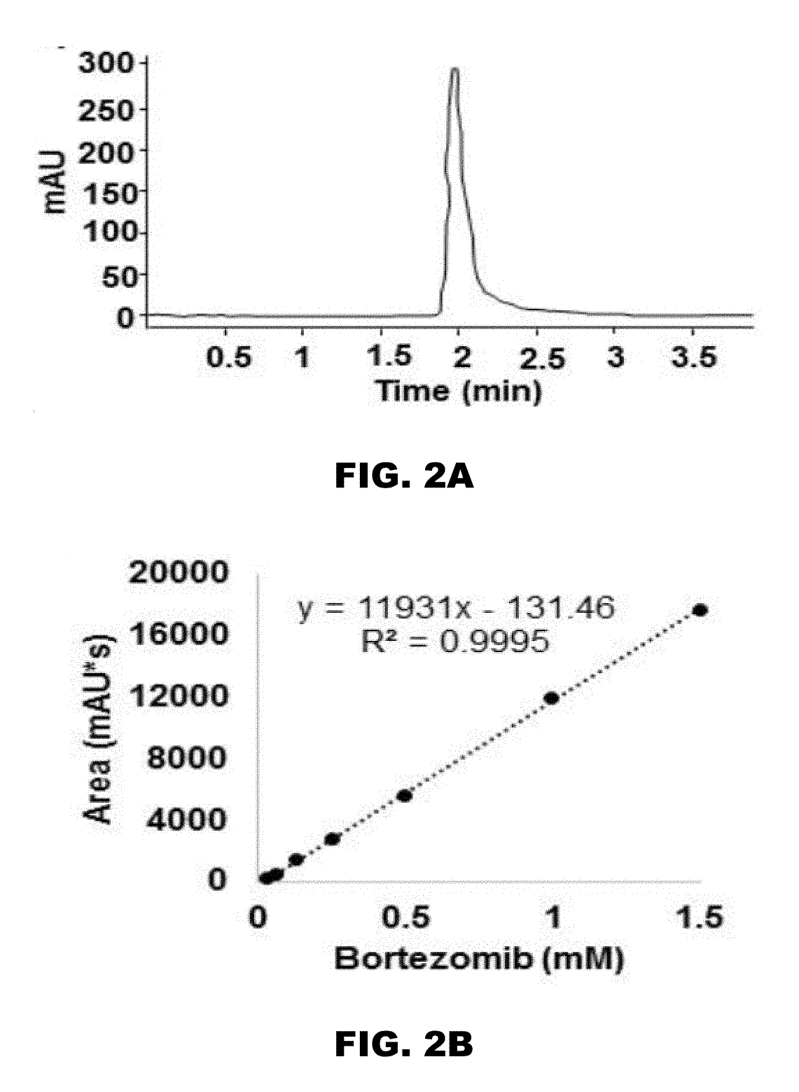

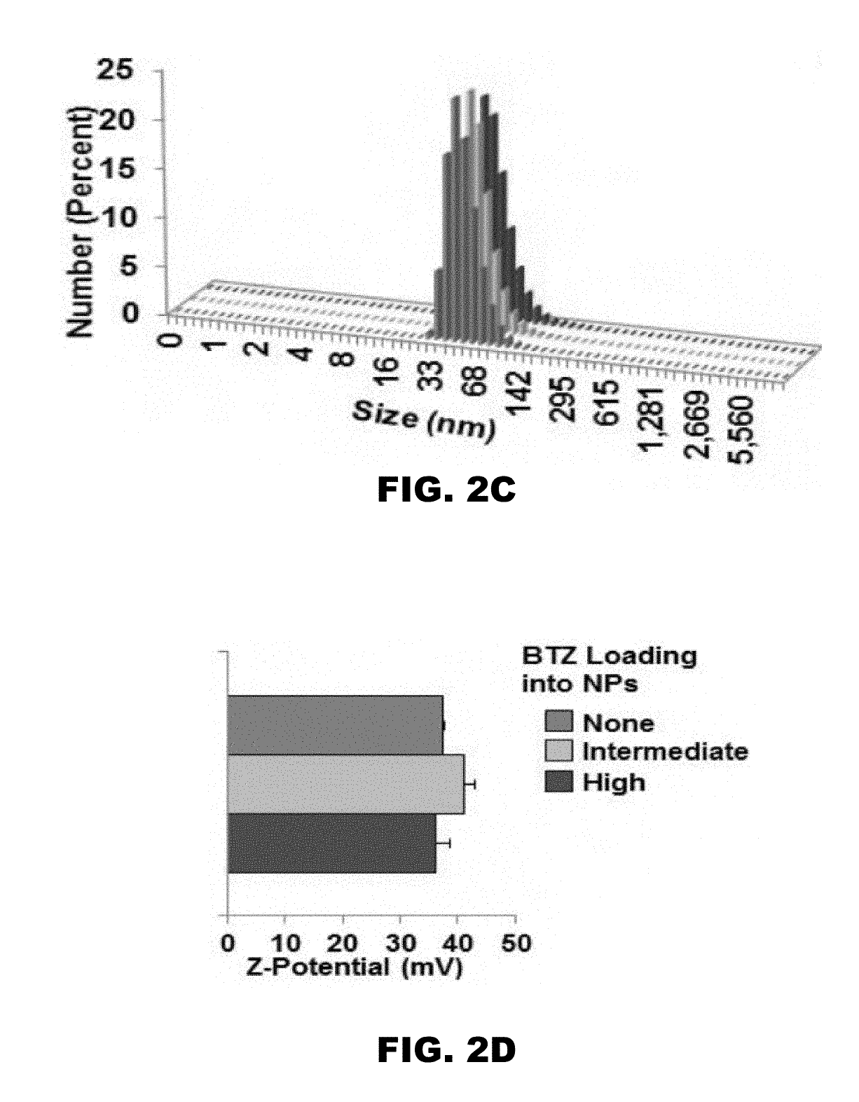

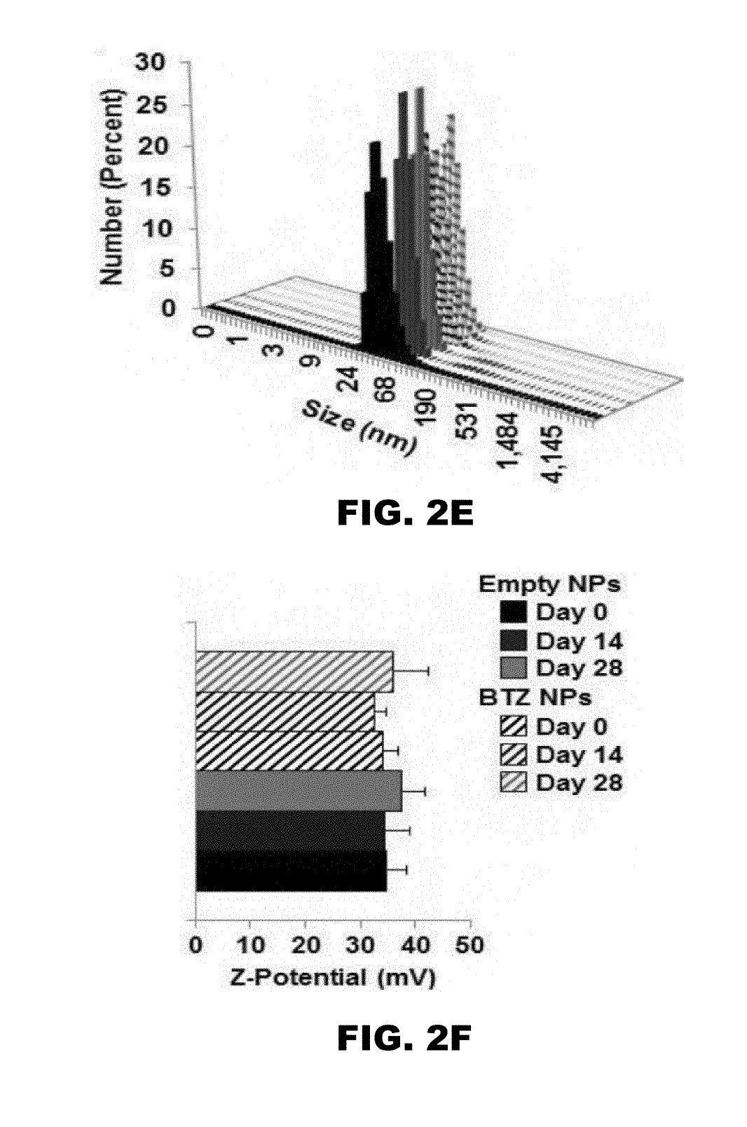

For evaluating the encapsulation efficiency of BTZ, a reverse phase HPLC assay was developed. BTZ had a retention time of 2 min (FIG. 2A) and illustrated a linear dynamic range in the concentration range between 0 and 1.5 mM with a R2=0.9995 (FIG. 2B). Anti-CD38 chitosan NPs were incubated for 1 h with intermediate (50 μM) or high-dose (1 mM) BTZ. Encapsulation efficiency was calculated using the AUC values for BTZ-loaded chitosan NPs. The encapsulation efficiency of intermediate and high-dose BTZ was 10.57±4.90 and 9.67±2.45 when incorporated after NPs crosslinking, and 85.31±6.24 and 83.32±5.22 when incorporated previous crosslinking in the crosslinker solution, respectively. In addition, the effect of BTZ loading inside of anti-CD38 chitosan NPs on size and stability was measured and we found that neither size (FIG. 2C) nor ζ-potential (FIG. 2D) were affected by intermediate or high BTZ loading. We further tested the size and the stability of the particles as a function of storage time to optimize their stability for biological use, and we found that the size of empty and BTZ loaded NPs did not change over a month when particles were kept at 4° C. (FIG. 2E), and the stability (FIG. 2F) was not changed in the same period of time.

Experiment 2. In Vitro Drug Release from Anti-CD38 Chitosan NPs to Different Environments.

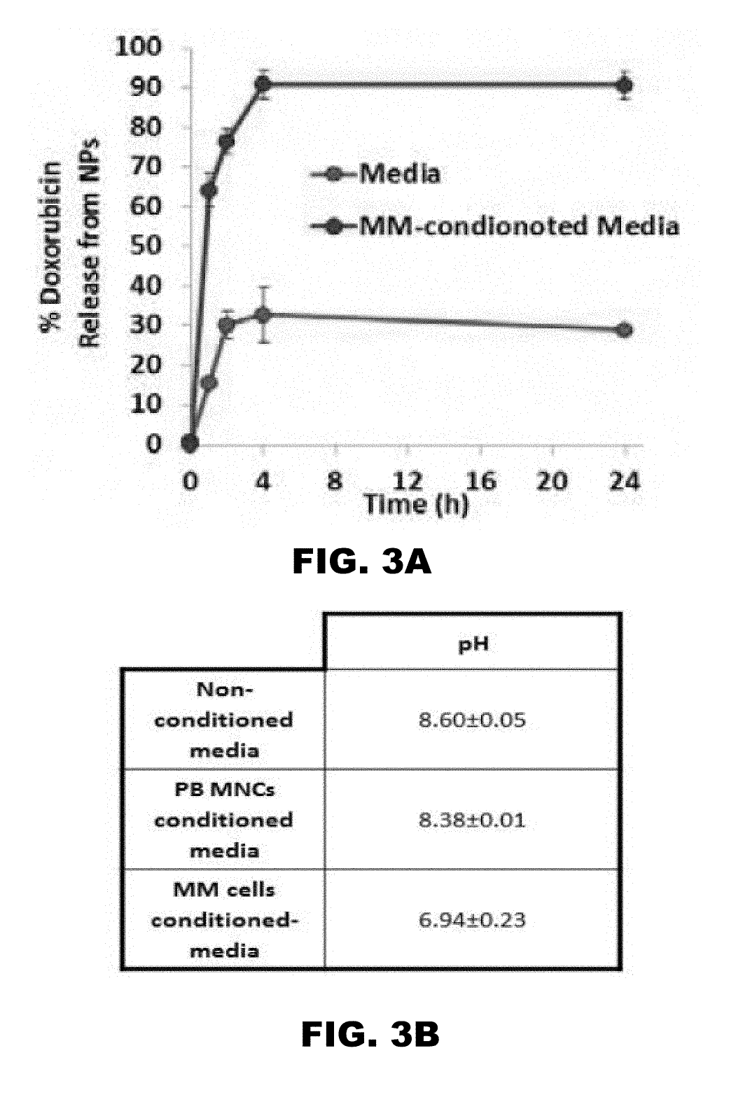

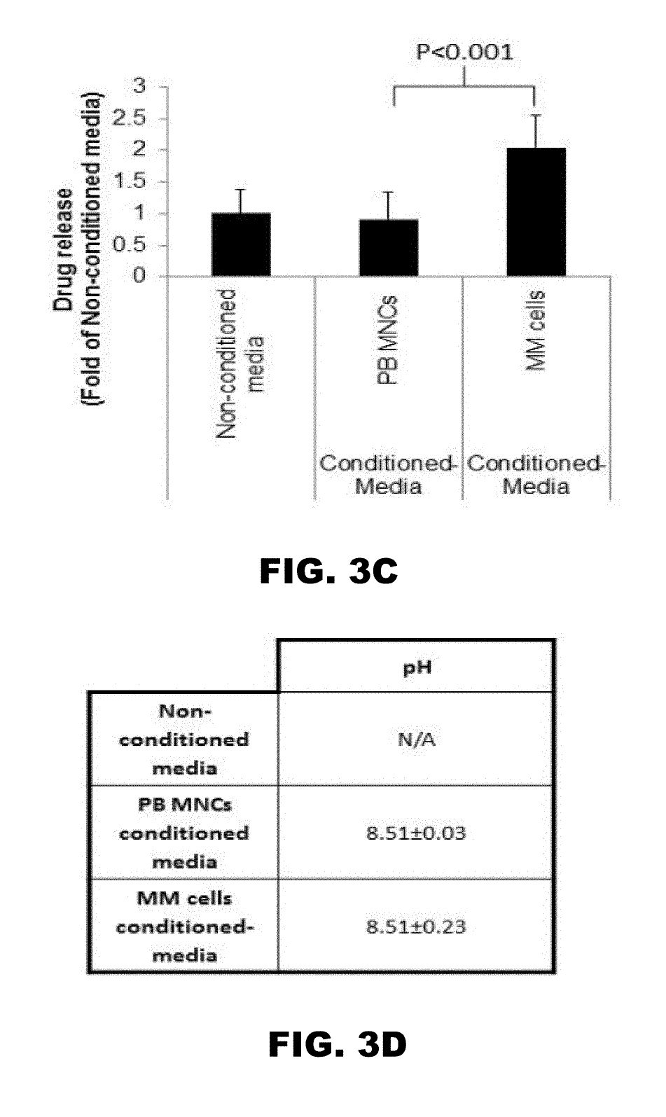

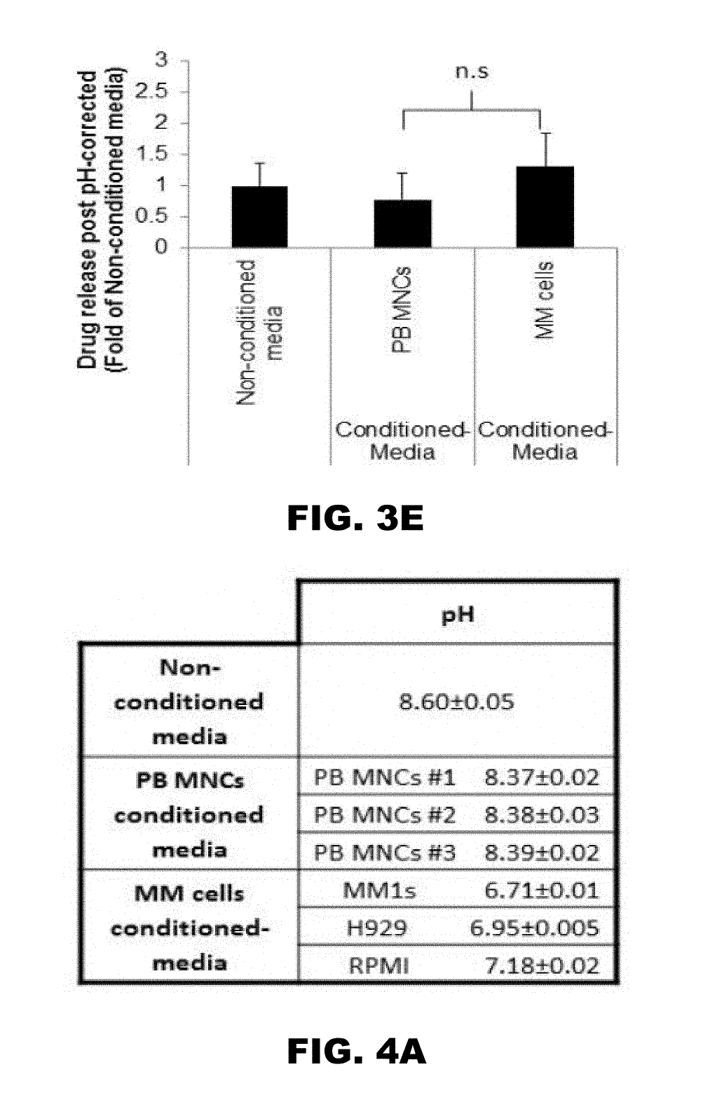

We hypothesize that the acidic tumor environment induces a rapid swelling of the chitosan NPs due to the free amine groups in the chitosan and faster release of the drug compared to neutral pH in the regular media (representing blood and normal tissues). First, pH of MM cell conditioned media was tested at different time points. We found that 72 h of MM cell culture generate acidic tumor microenvironment close to the pH tumor environment cited in the literature around 6.5-7.1 [27-29]. The pH of the conditioned media was measured after 72 h of culture and found a significant decreased in pH in MM-conditioned media (6.94±0.23) compared to normal PB MNCS-conditioned media (8.38±0.01) and non-conditioned media (8.6±0.05) (FIG. 3B). The in vitro release of a model fluorescent drug (doxorubicin) from anti-CD38 NPs was analyzed in the conditioned media of MM cells and normal PB MNCs. The acidic tumor microenvironment induced a 2-fold increase release of drug from the anti-CD38 NPs compared to non-conditioned media and normal PB MNCS-conditioned media (FIG. 3C).

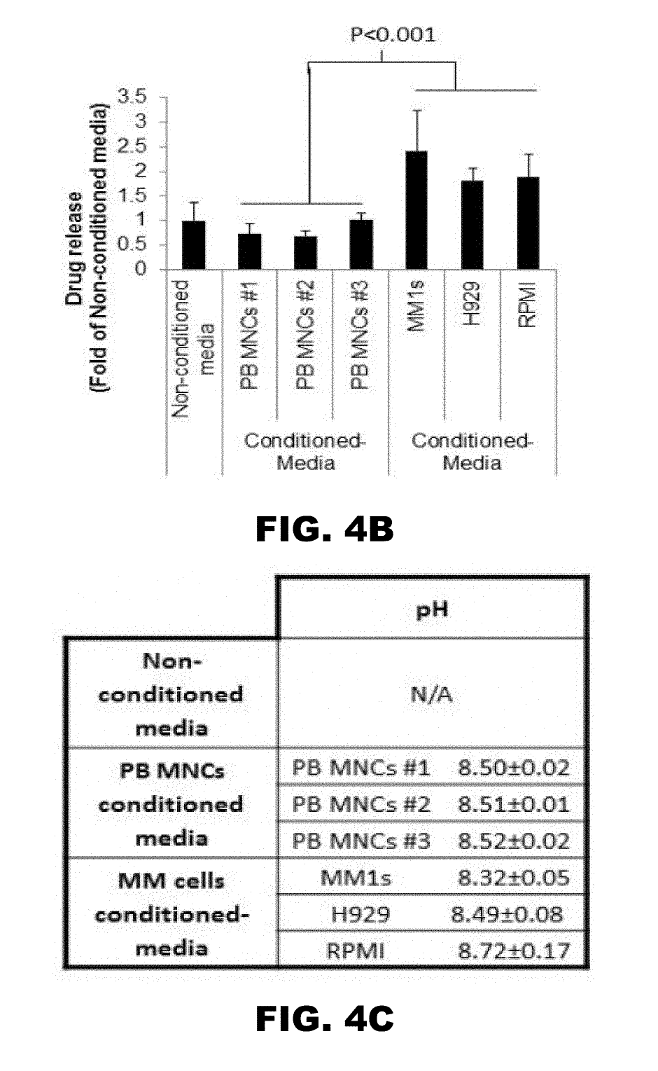

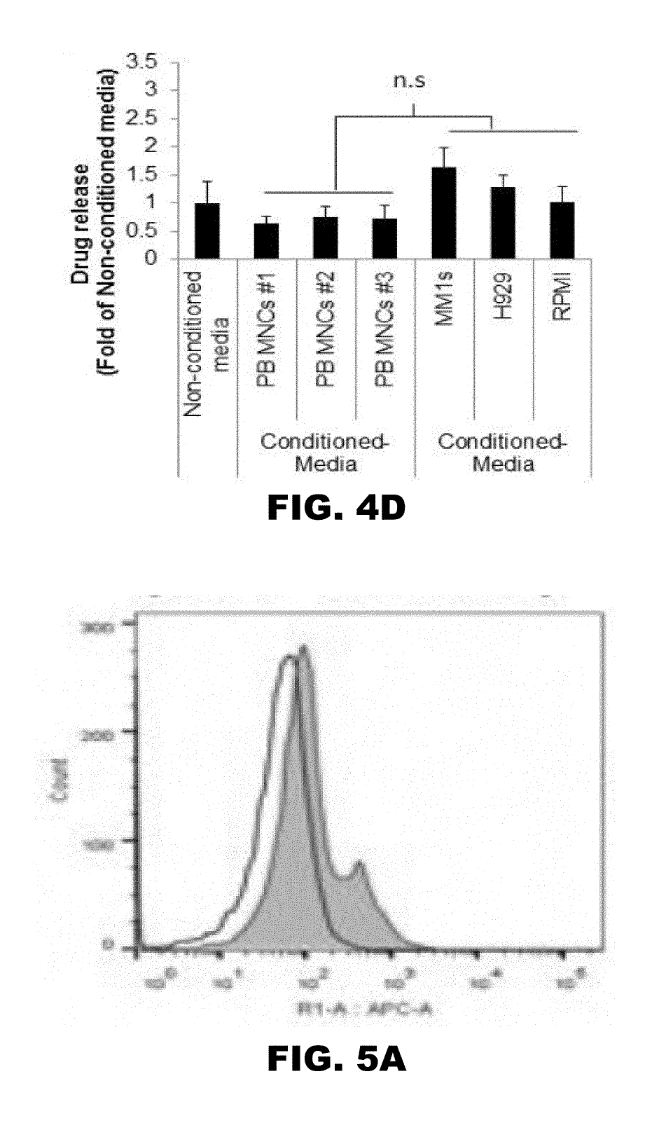

To corroborate that the increase drug release was due to the change in pH, all the conditioned media were corrected to the pH of non-conditioned media and drug release was evaluated again. Note that the pH-corrected was around 8.51 for both MM-conditioned media and normal PB MNCS-conditioned media (FIG. 3D). The release in the pH-corrected conditioned media (MM and PB-MNCs) were non-significantly different than in non-conditioned media (FIG. 3E), reflecting that the acidic tumor environment induced faster release of the drug compared to neutral-basic pH (8.5). In addition, it's important to note that while the conditioned media from PB MNCs was very consistent (8.38), the conditioned media from MM cells showed more variability in different cell lines (FIG. 4A), with indirect correlation where the lower the pH the more drug is released from the anti-CD38 NPs (FIG. 4B). An effect that can be reversed by correcting the pH of the media (FIG. 4C), with proportional reduction of drug release to a level of non-significant difference compared to the conditioned media of normal PB MNCs (FIG. 4D).

### Experiment 3. Kinetics of Binding of Anti-CD38 Chitosan NPs to MM Cells.

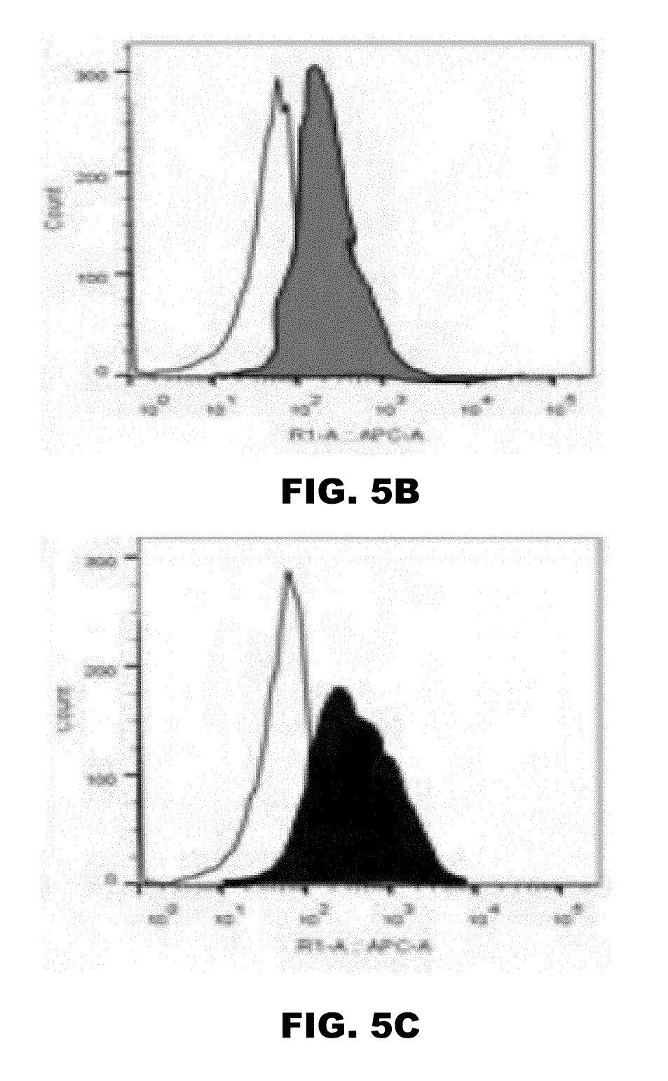

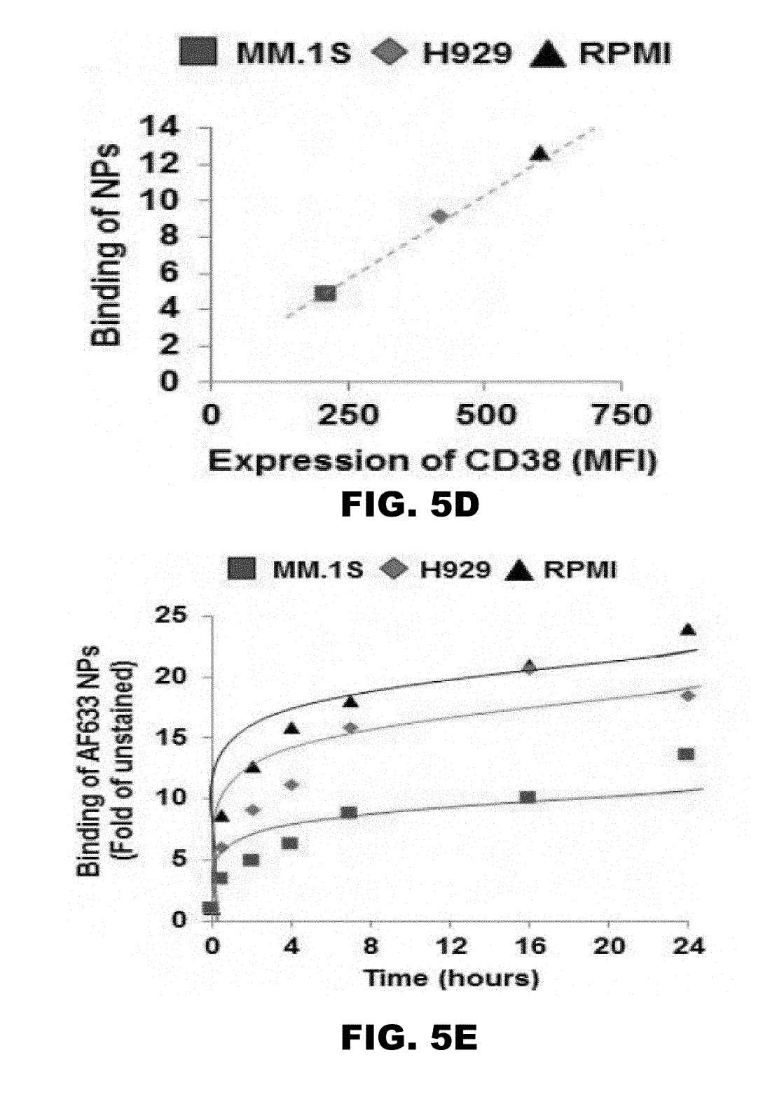

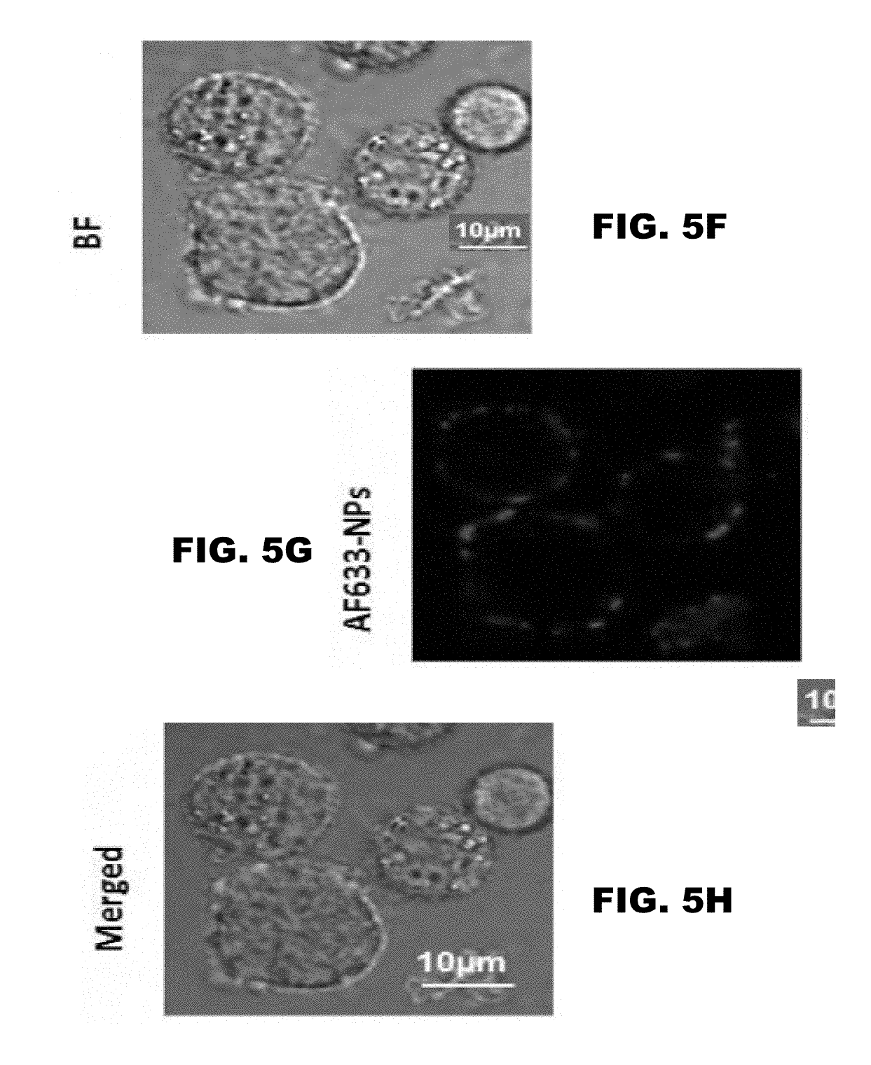

First, we tested the expression of CD38 in three MM cell lines and found that although all the cell lines showed a high expression compared to the corresponding isotype, the different cell lines showed different CD38 expression (FIG. 5A-C). Then, we tested the binding of anti-CD38 chitosan NPs at 2 h with the CD38 expression in these cell lines, and found that the binding of the particles was directly correlated with the level of expression of CD38 (FIG. 5D).

Next, we determined the kinetics of binding for the anti-CD38 chitosan NPs in the three MM cell lines over a period of time of 24 h. We found that the binding is very fast and in a short period of a round 2-4 h the binding reach a plateau (FIG. 5E). FIG. 5F-H demonstrated the binding of the anti-CD38 chitosan NPs (red) to the surface MM.1S cells at 2 h post-treatment. In addition, we study the potential dissociation of the anti-CD38 chitosan NPs from the MM cells after binding, and found that the particles did not dissociate from the MM cells over 24 h of incubation (FIG. 5I).

### Experiment 4. Specificity of Binding of Anti-CD38 Chitosan NPs to MM Cells In Vitro.

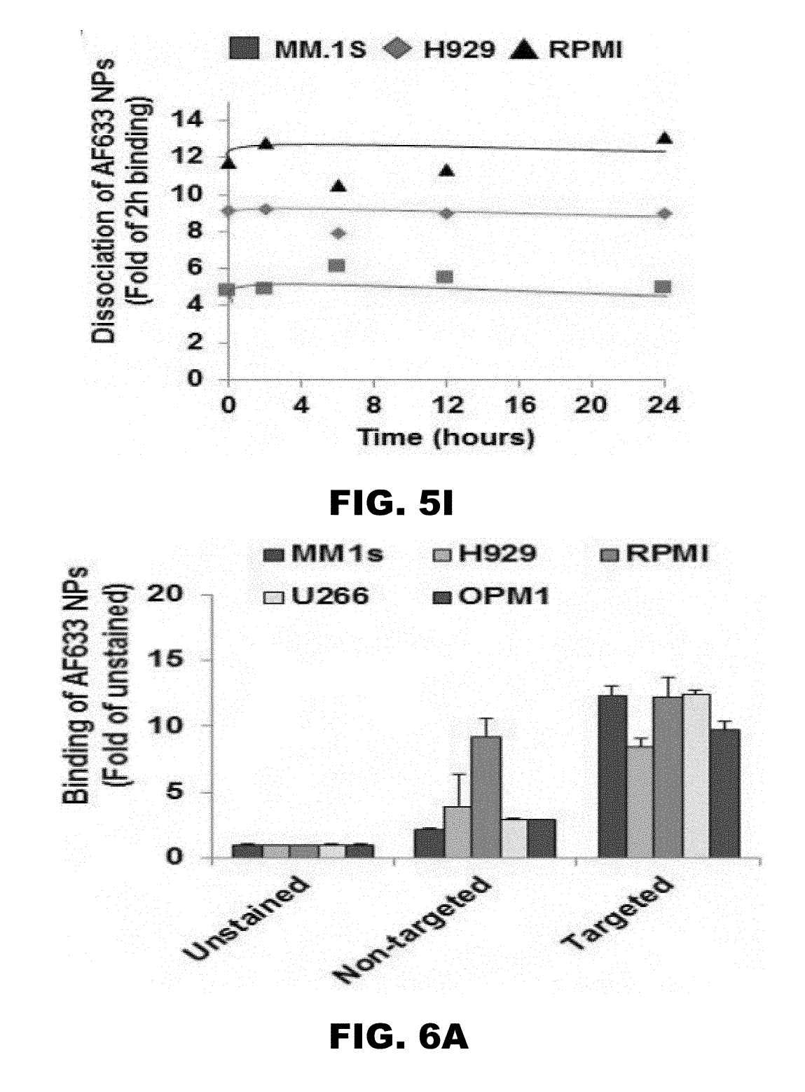

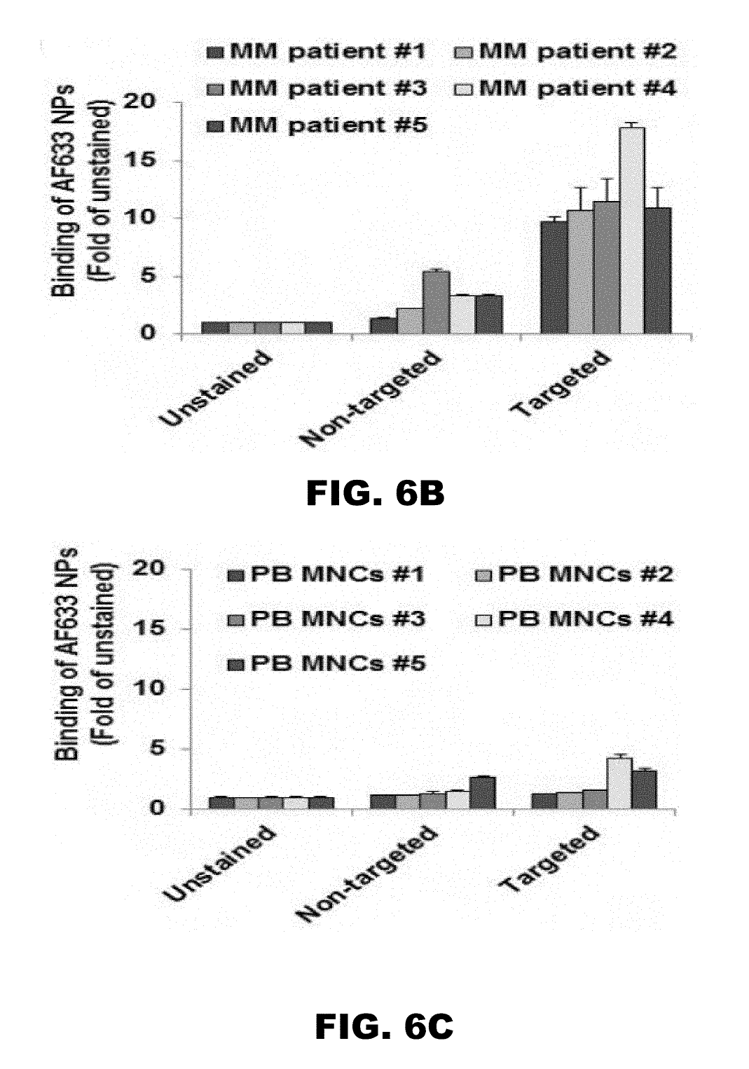

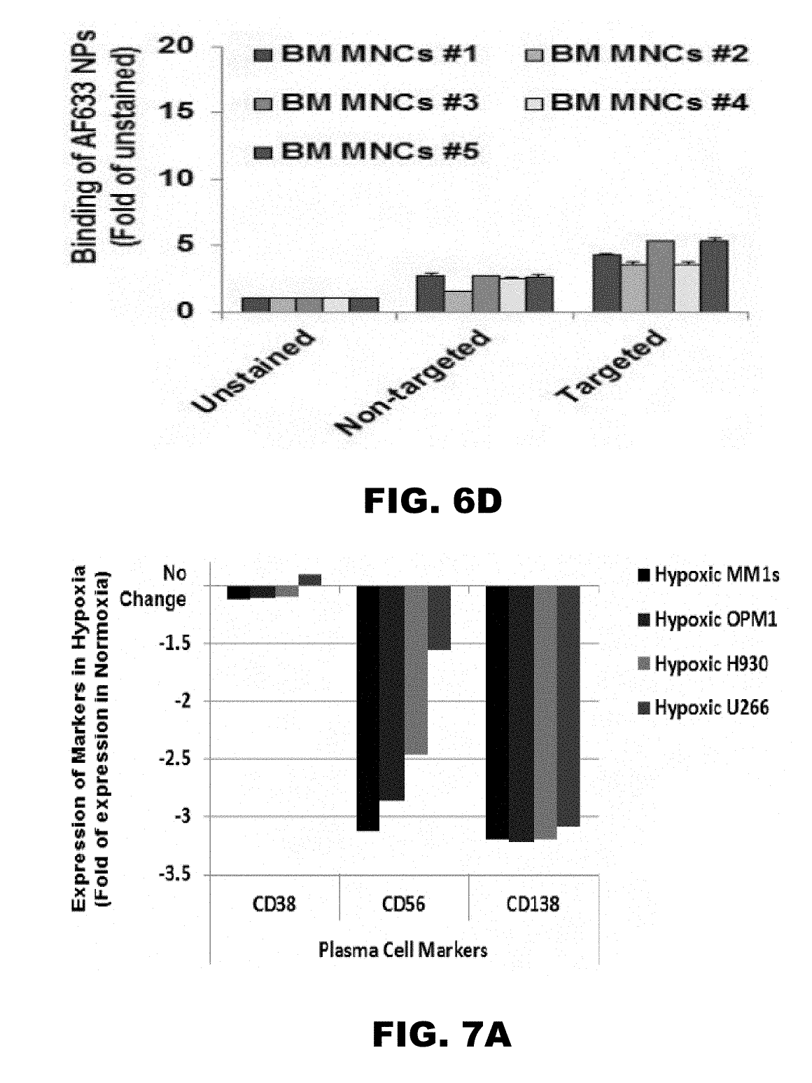

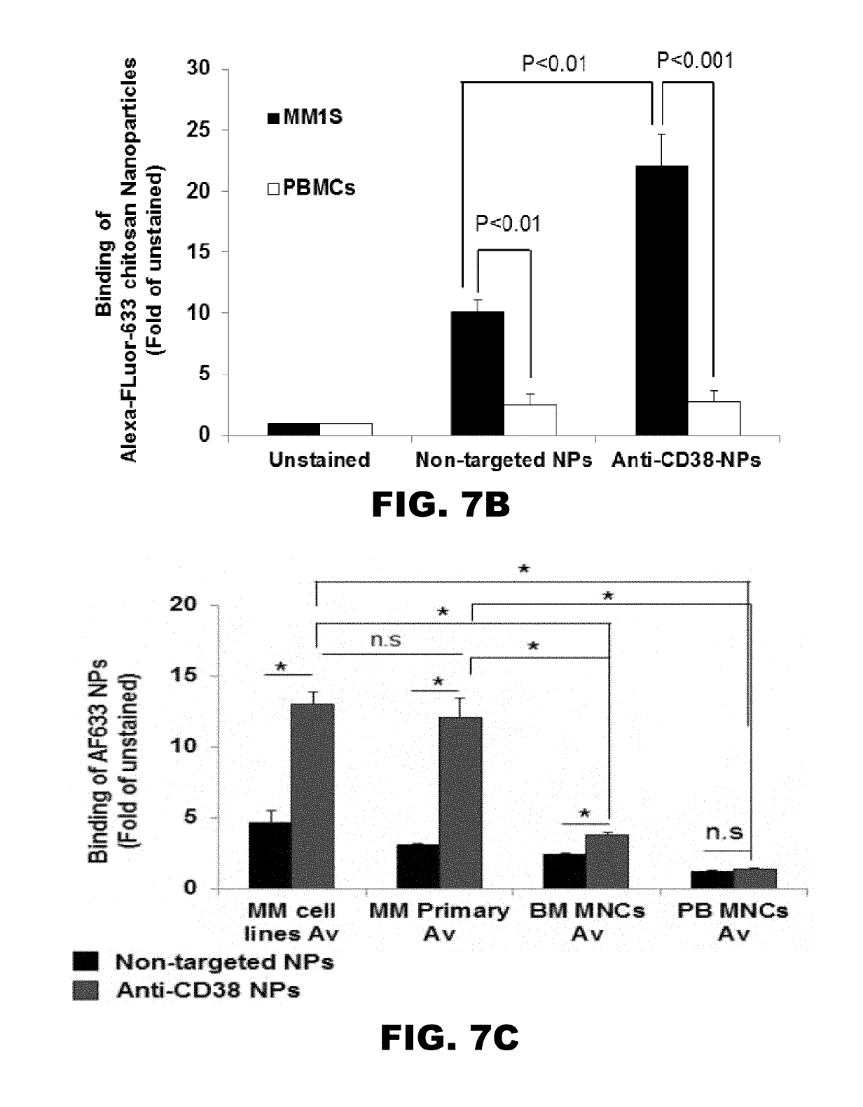

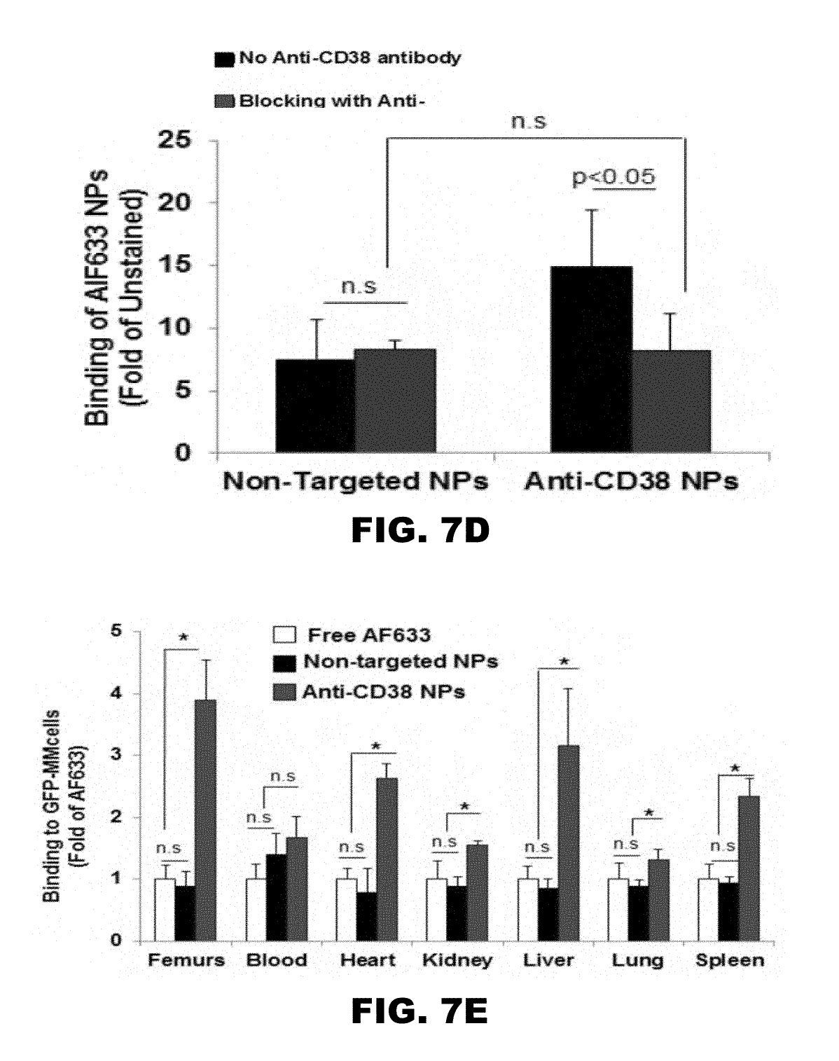

The binding of anti-CD38 chitosan NPs to five MM cell lines and primary MM cell isolated from five MM patients was significant (3-fold) higher than the binding in non-targeted NPs (FIG. 7C). The binding of the anti-CD38 chitosan NPs was highly consistent in the cell lines and the primary samples, while the binding of the non-targeted NPs to the different cell lines was more variable (FIGS. 6A and B). For normal controls, we used mononuclear cells isolated from the peripheral blood of five normal subjects (PB MNCs,) and from the BM of five normal subjects (BM MNCs). We found that the binding of the anti-CD38 chitosan NPs to the normal PB MNCs and BM MNCs was significantly (4-folds and 10-folds, respectively) lower than the binding to MM cells (FIG. 7C). The binding of both the anti-CD38 chitosan NPs and the non-targeted NPs was constantly low with little variability between samples of PB MNCs (FIG. 6C) and BM MNCs (FIG. 6D).

### Experiment 5. Specificity of Binding of Anti-CD38 Chitosan NPs to MM Cells In Vivo.

We further study the specificity of anti-CD38 NPs binding to MM cells in vivo. MM-bearing mice were injected with free AF633 dye, non-targeted AF633-NPs and anti-CD38 AF633-NPs, and its binding to MM cells was tested in different organs. We found that free AF633 and non-targeted particles have the same random distribution (no significance differences) in the MM cells (GFP+) of all the organs. However, the binding of the anti-CD38 chitosan NPs was significantly higher in MM cells in the BM (femurs) and other extramedullary organs (heart, kidney, liver, lung and spleen), with around 4.5-fold increase of targeted compared to non-targeted NPs in the femurs and 1.5 to 3-fold increase in the other organs (FIG. 7E). In the blood, we found that there was no significance difference between the binding of the anti-CD38 chitosan NPs compared to non-targeted and free AF633, probably due to same accessibility of NPs (targeted and non-targeted) to the cells in the circulation.

### Experiment 6. Effect of Bortezomib-Loaded Anti-CD38 Chitosan NPs on Proliferation, Cell Cycle, and Apoptosis in MM Cells.

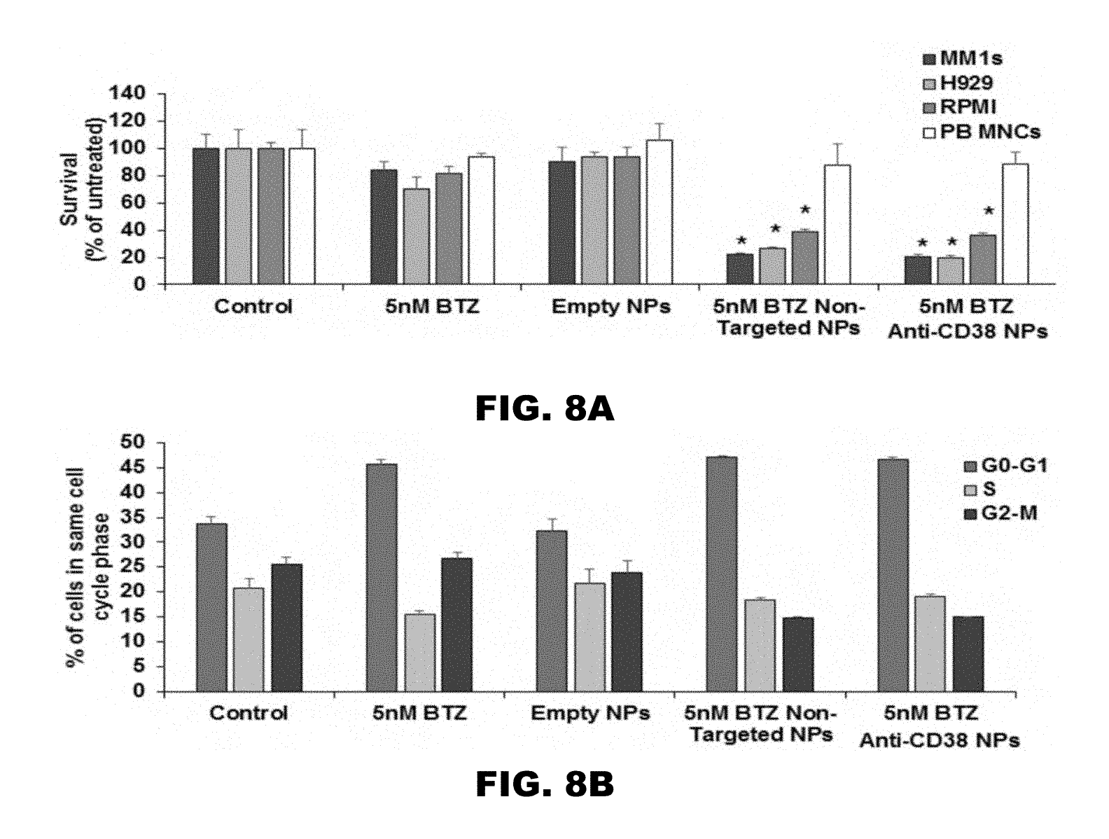

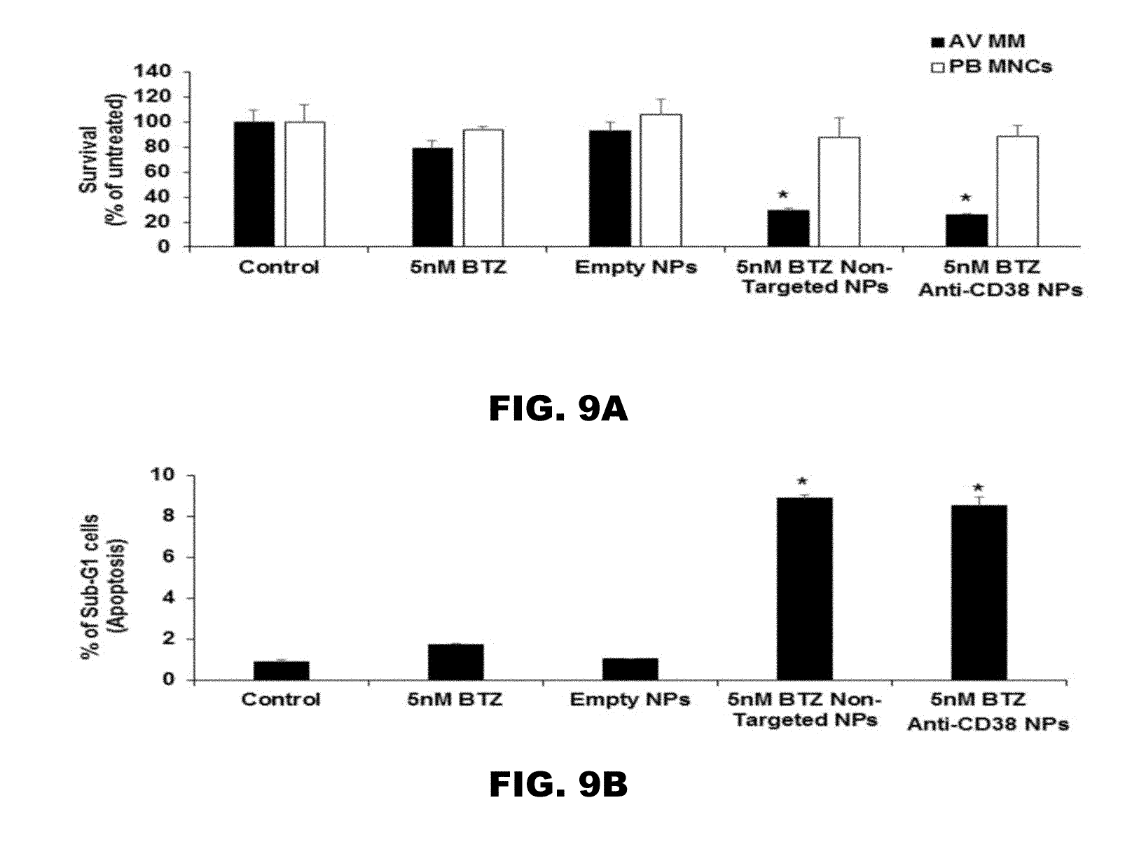

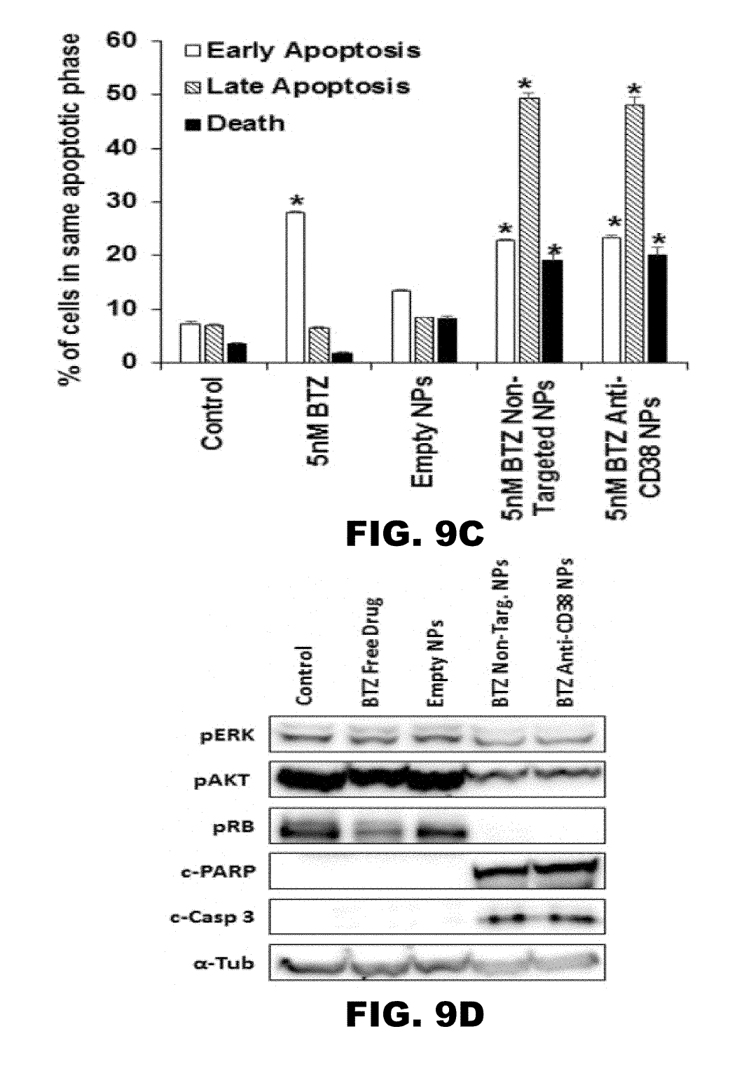

The effect of vehicle (control), BTZ as a free drug (5 nM), empty chitosan NPs, non-targeted chitosan NPs loaded with BTZ amounts to 5 nM, and anti-CD38 chitosan NPs loaded with BTZ amounts to 5 nM for 48 h on proliferation of MM1s, H929, RPMI cells and PB MNCs was analyzed by MTT. We found that BTZ 5 nM as a free drug have a modest effect (20% killing) on MM cells with no effect on normal mononuclear cells from peripheral blood (PB MNCs). While empty chitosan particles showed no effect on survival of MM cells or PB MNCs, BTZ encapsulated in non-targeted and anti-CD38 chitosan NPs (5 nM) had a robust effect (70% killing) in MM cells with no effect on PBMNCs (FIG. 9A). The effects on individual cell lines was consistent with low variability (FIG. 8A).

We further investigated the same therapy regimens on cell cycle and apoptosis of H929 cells. We found that BTZ as a free drug induced G0-G1 arrest, which was demonstrated also by BTZ encapsulated in non-targeted and targeted NPs, while empty chitosan NPs showed no effect (Fig. S4.B). However, BTZ encapsulated in non-targeted and targeted NPs revealed an important increase in subG1 cells (apoptosis) compared to empty NPs and free BTZ (FIG. 5.B). BTZ as a free drug induced increase early apoptosis and no effect on late apoptosis and death, non-targeted and targeted NPs showed a significant increase in the fraction of early and late apoptosis, and cell death compared to free drug (FIG. 9C).

Finally, these effects were confirmed on survival, cell cycle, and apoptosis signaling molecules and we found that BTZ encapsulated in non-targeted and targeted NPs suppressed more potently the phosphorylation levels pAKT and pERK, induced down-regulation of the expression of protein involved in cell cycle transition, such as pRb, and increased cleaved caspase 3 and PARP compared to free drug, while vehicle control and empty NPs did not have an effect (FIG. 9D).

### Experiment 7. Enhanced Bortezomib Uptake Lead to Improved Proteasome Activity Inhibition.

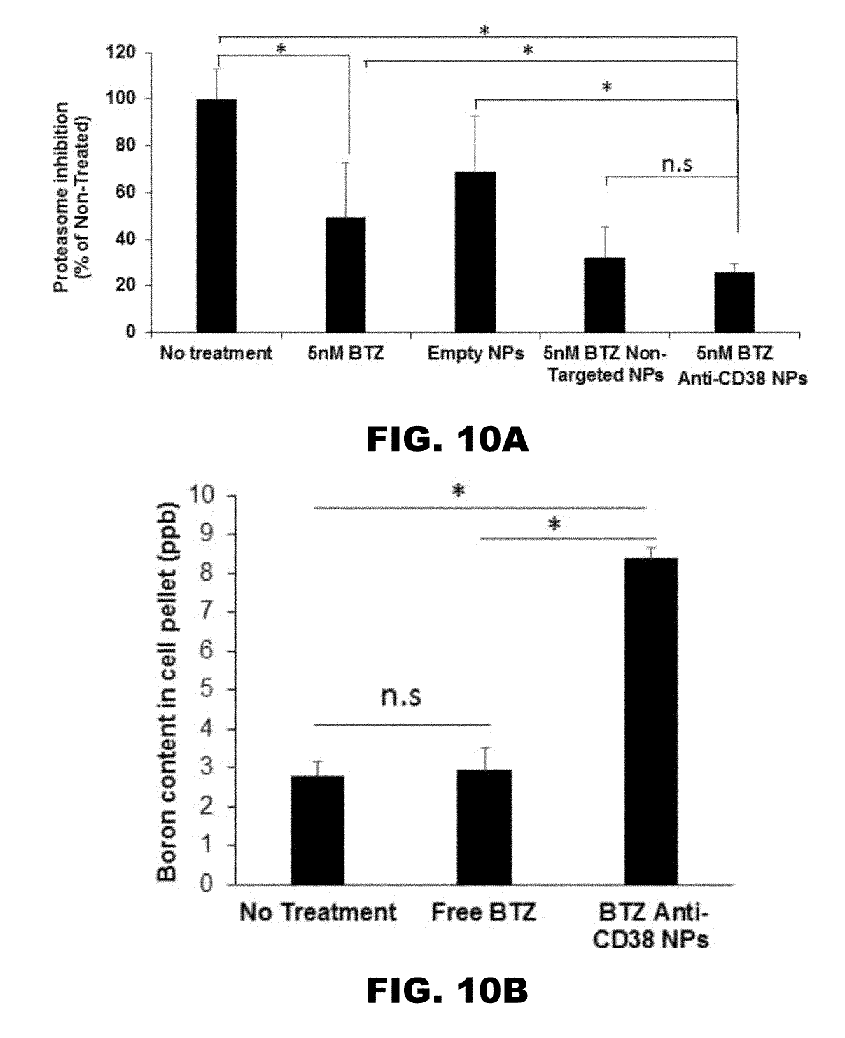

To explain the enhanced effect of the encapsulated BTZ, we tested the effect of the BTZ encapsulation in the NPs on its ability to inhibit the proteasome activity. The effect of BTZ loaded anti-CD38 NPs and non-targeted NPs on proteasome activity inhibition was compared to free BTZ. We found that BTZ as a free drug have a moderate effect (50% inhibition) on MM cells; while BTZ encapsulated in non-targeted and targeted NPs had a more robust effect (68% and 75% inhibition, respectively) (FIG. 10A). These results are in agreement with our previous results showing a more robust effect of the NPs on survival and apoptosis of MM cells compared to free drug (FIG. 9A-D).

To explain the effect on the proteasome activity, we tested the effect of BTZ encapsulation in NPs on its accumulation in the cells, as reflected by the boron content of the cells using elemental analysis of the cells by ICP-OES. Elemental analysis showed that the boron content of cells treated with BTZ encapsulated in anti-CD38 NPs showed a significant (3-fold) higher than the boron content after treatment with free BTZ (FIG. 10B). It is worth notice that the accumulation of BTZ as a free drug was very similar to the levels measured in control, reflecting clearly the low uptake of free BTZ into the cells.

### Experiment 8. Enhanced Proteasome Activity Inhibition by Anti-CD38 Chitosan NPs Endocytic Internalization.

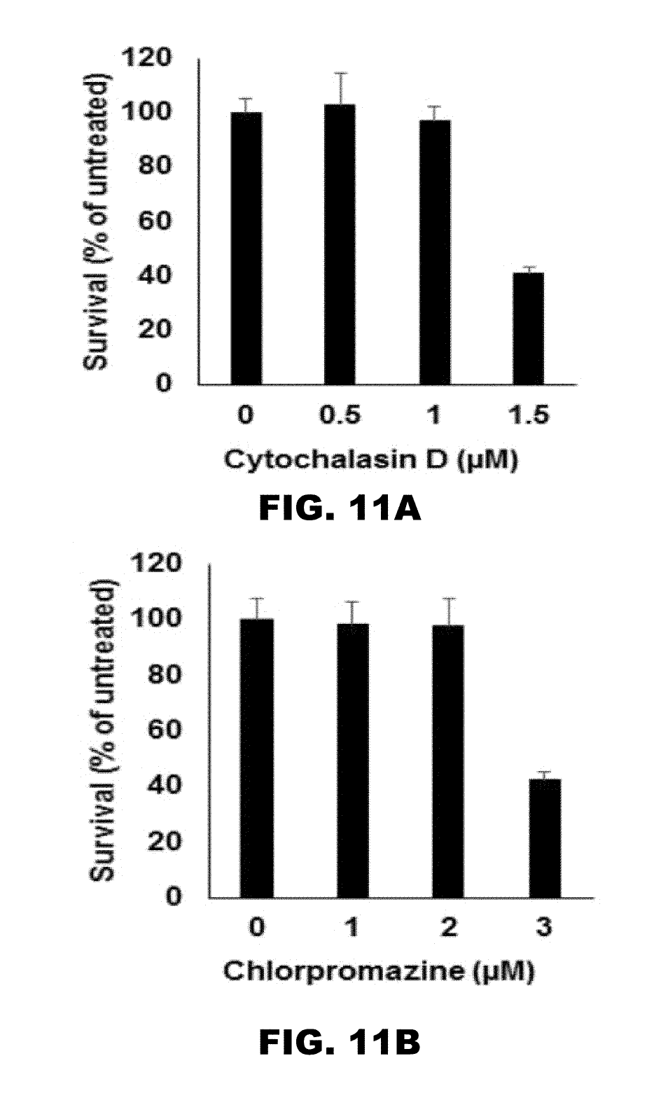

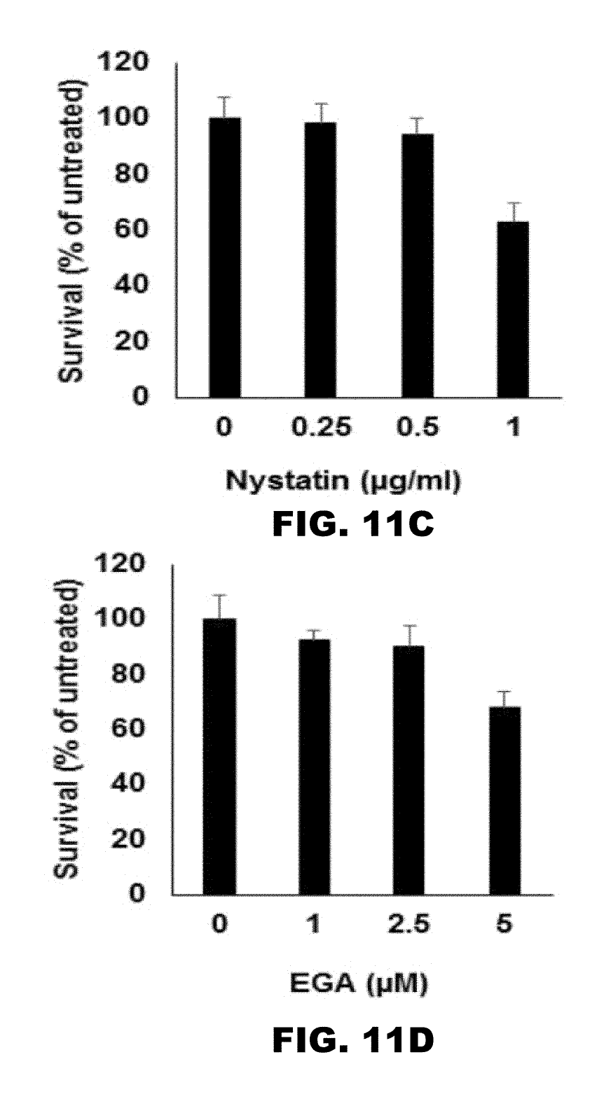

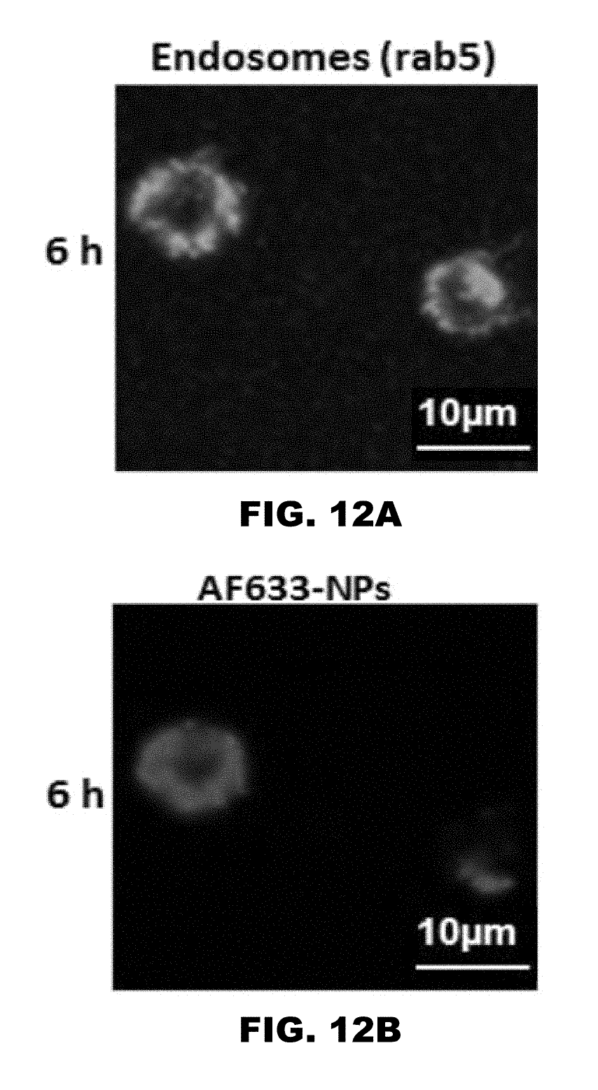

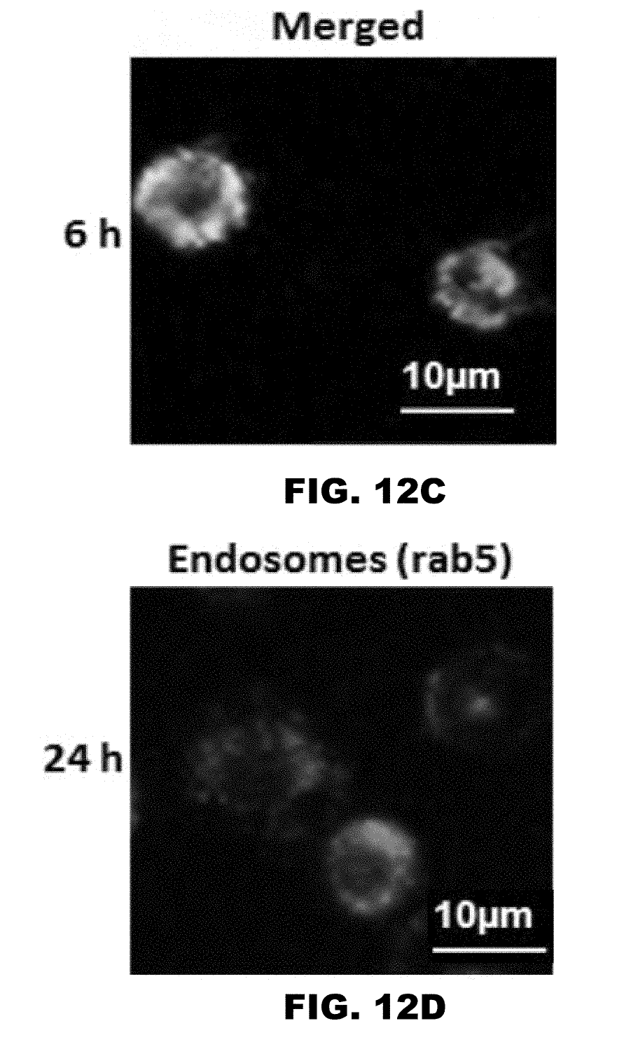

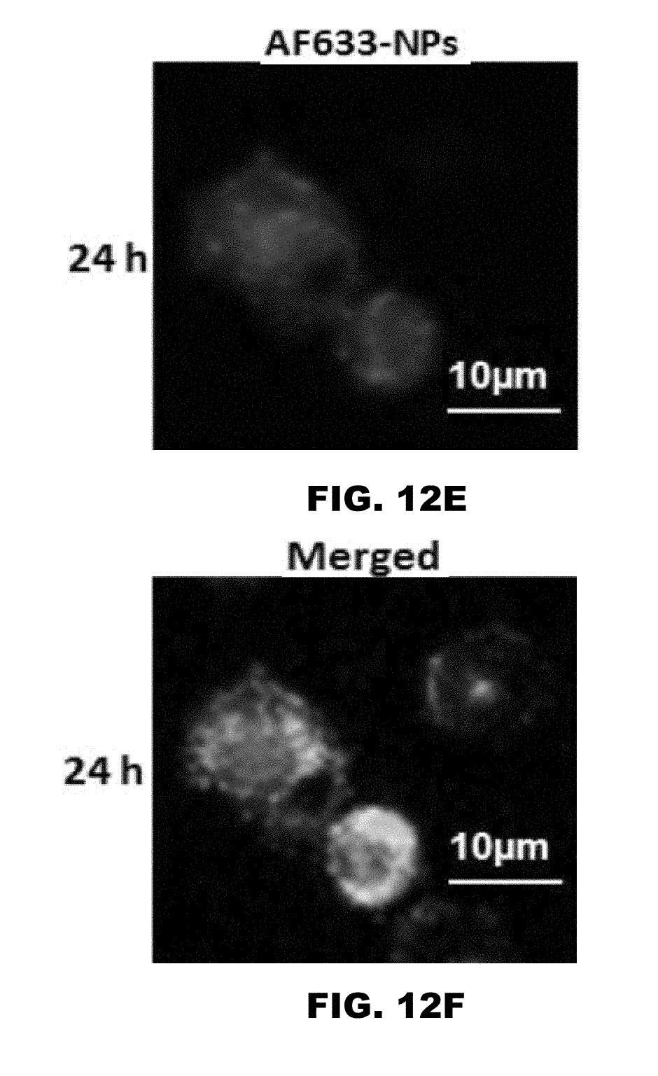

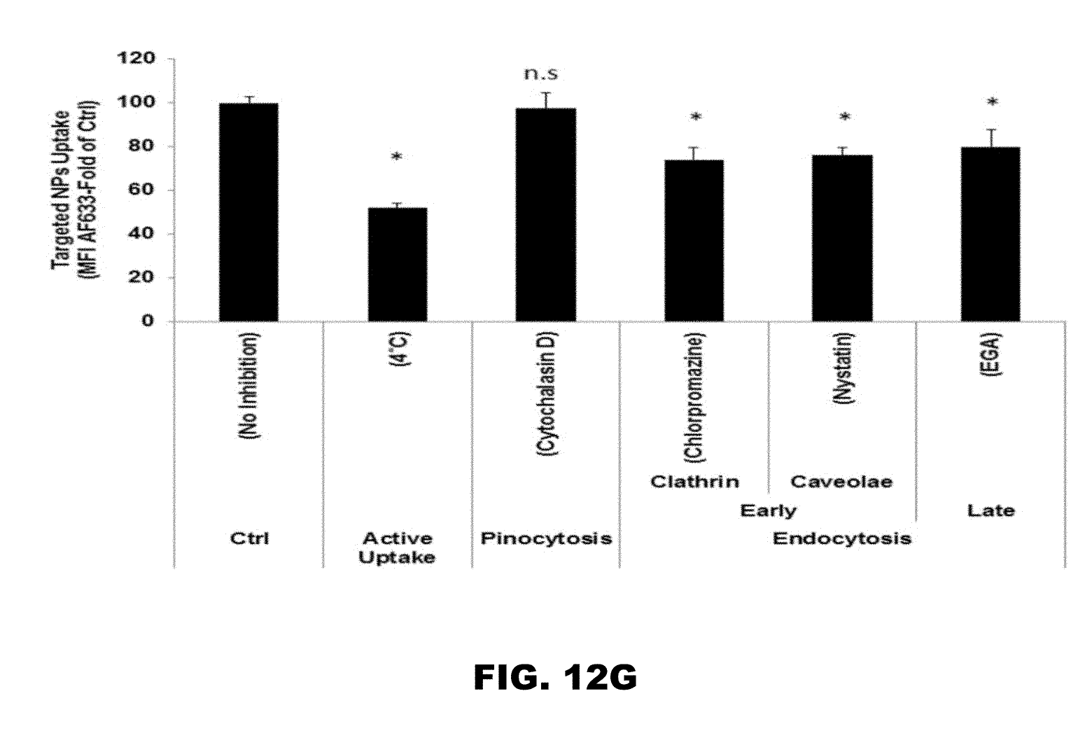

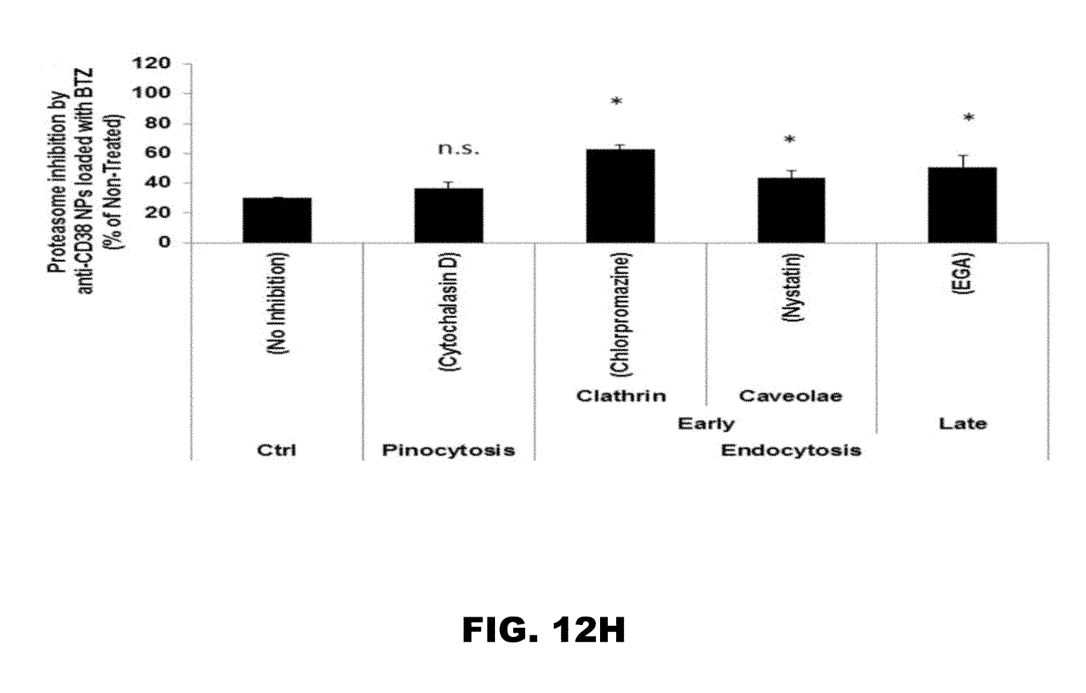

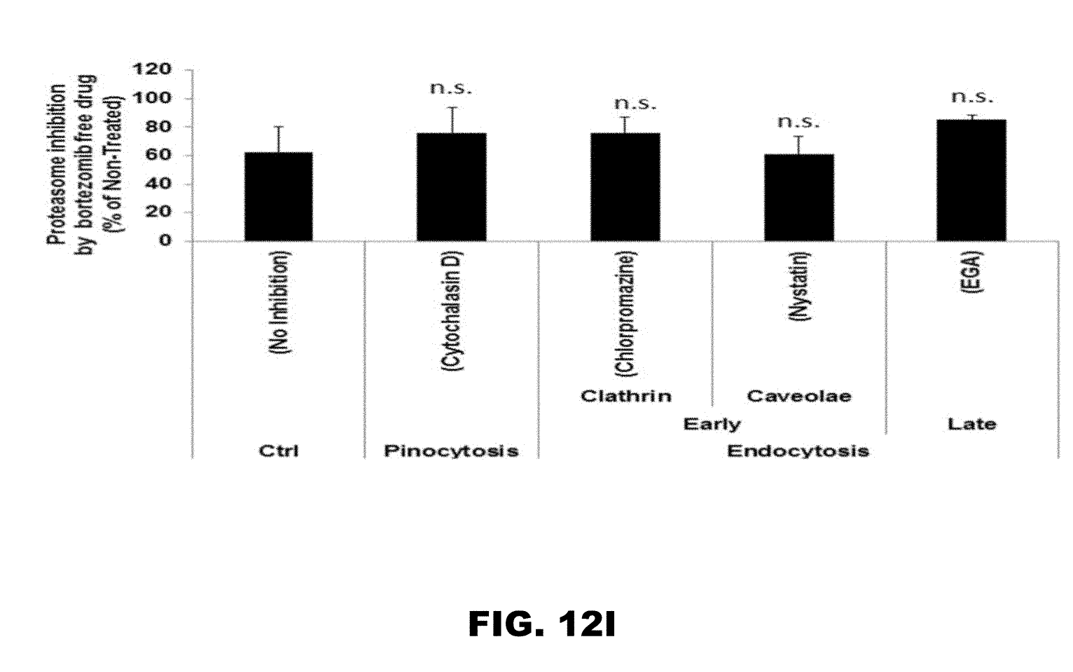

To understand the mechanism of the uptake of anti-CD38 chitosan NPs we tested the sub-cellular co-localization of the NPs with the endosomal markers (rab5+). We found that anti-CD38 chitosan NPs (red) were taken up by the MM cells via endocytosis and transferred into early endosomes (rab5+) at 6 h and still co-localization was detected by 24 h (FIG. 12A-F),Incubation of MM cells at 4° C. before treatment with anti-CD38 NPs significantly (50%) decreased their uptake, indicating it is an active uptake process (FIG. 12G). To further investigate the mechanism of active uptake of the anti-CD38 NPs, we tested the contribution of macropinocytosis, early endocytosis pathways (mediated by clathrin or caveolae), and late endocytosis internalization, by specific inhibitors of the different uptake routs [30, 31]. We preliminary screened all the inhibitors on survival of MM cells to determine a concentration that does not induce cell death. Pre-incubation at 37° C. with cytochalasin D 1 μM (FIG. 11A), chlorpromazine 2 μM (FIG. 11B), nystatin 0.5 μg/ml (FIG. 11C), and EGA 2.5 μM (FIG. 11D) for 30 min was found to have no effect on cell survival. Then, we found that while inhibition of macropinocytosis (by Cytochalasin D) did not affect the uptake of anti-CD38 NPs, inhibition of early endocytosis clathrin-mediated (by chlorpromazine) or caveolae-mediated (by nystatin), and inhibition of late endocytosis (by EGA) significantly (about 25% each) decreased the uptake of the anti-CD38 NPs (FIG. 12G).

We further corroborated the effect of inhibition of each of the uptake routes on the proteasome inhibition induced by the BTZ-loaded anti-CD38 NPs. It was shown that MM cells treated with BTZ-loaded anti-CD38 NPs (without inhibition of the uptake routs) induced about 70% inhibition in the proteasome activity. While inhibition of macropinocytosis did not affect the proteasome inhibition induced by the BTZ-loaded anti-CD38 NPs, inhibition of the early (clathrin- and caveolae-mediated) and late endocytosis interfered with proteasome inhibition activity of the BTZ-loaded anti-CD38 NPs (FIG. 12H). We also confirmed that MM cells treated with free BTZ (without inhibition of the uptake routs) induced about 40% inhibition in the proteasome activity and demonstrated that the inhibition of macropinocytosis or endocytosis pathways led to any significant effect on proteasome activity (FIG. 12I).

### Experiment 9. Effect of Bortezomib-Loaded Anti-CD38 Chitosan NPs on Drug Resistance Induced by the Tumor Microenvironment.

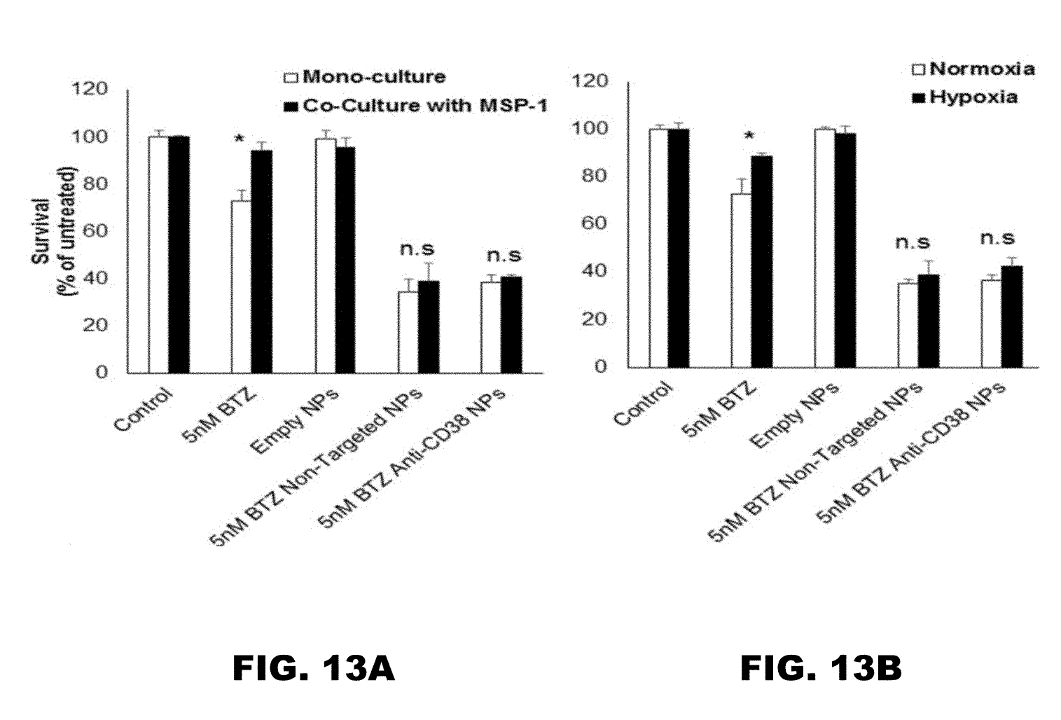

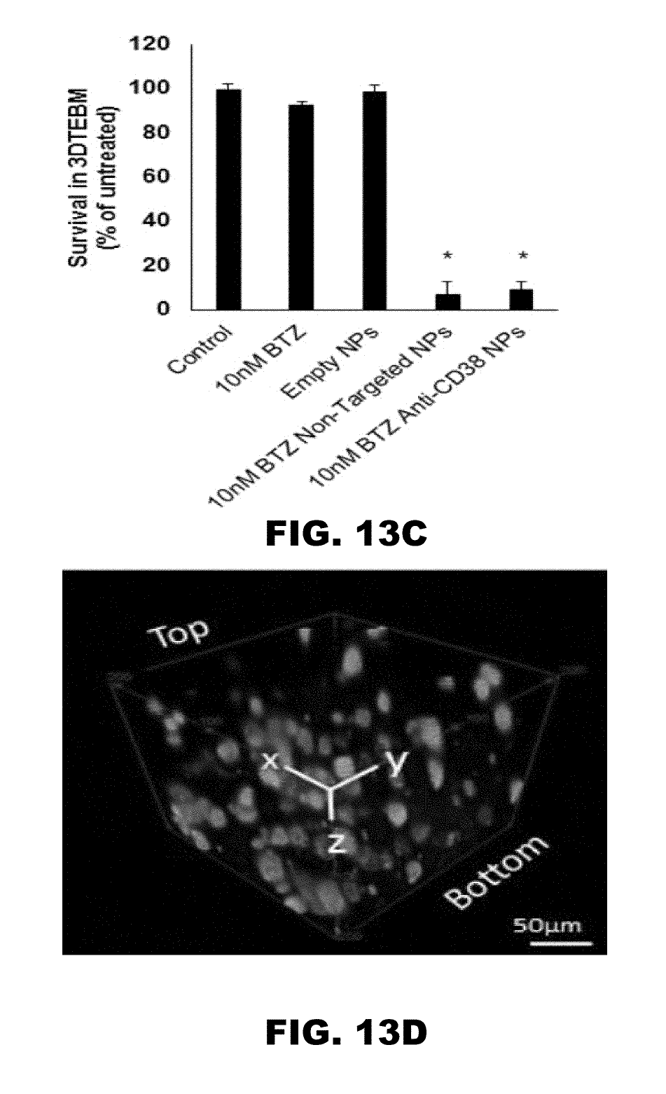

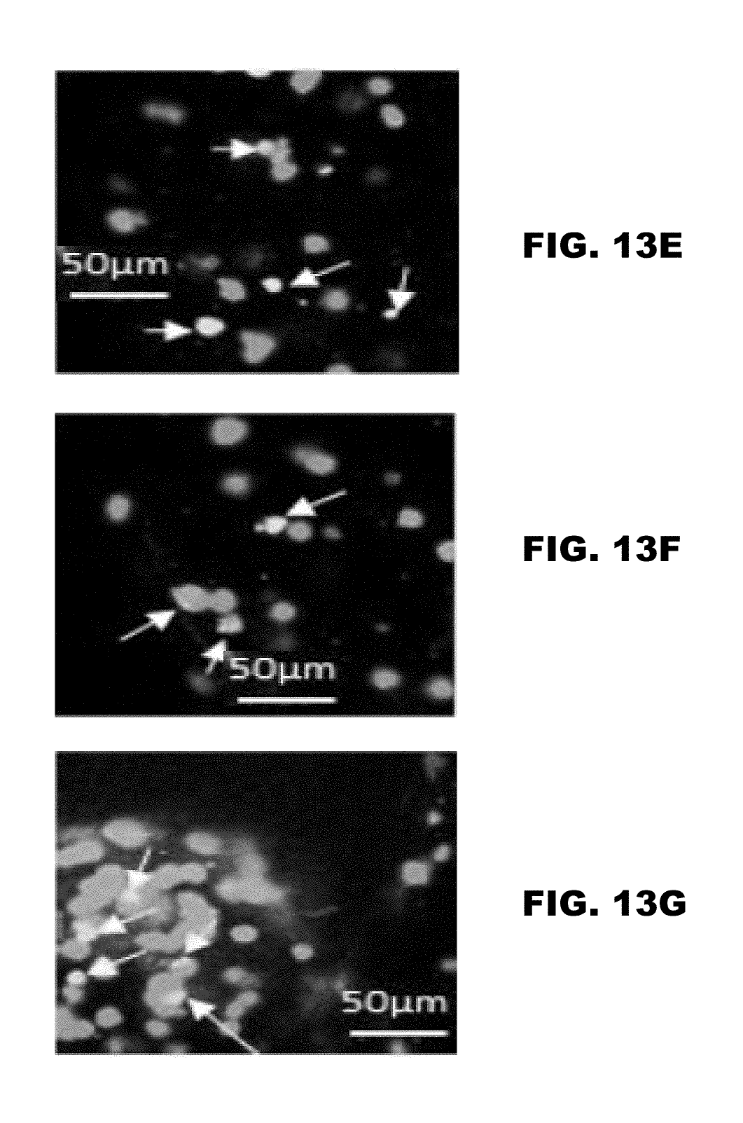

Stromal cells play a critical role in cell adhesion-mediated drug resistance (CAM-DR) in MM [17]. Co-culture of MM cells with stromal cells derived from MM patients induced resistance in the MM cells to treatment with BTZ (5 nM) as a free drug, compared to mono-culture of MM cells alone. In contrast, encapsulated BTZ in anti-CD38 NPs induced a significantly more profound killing effect in the MM mono-culture, and overcame the stroma-induced resistance in MM cells (FIG. 13A).

Similarly, hypoxia in the microenvironment was shown to play an important role in cell drug resistance in MM [25]. Incubation of MM cells in hypoxic conditions (1% O2) induced resistance in the MM cells to treatment with BTZ (5 nM) as a free drug, compared to MM cells cultured in normoxic conditions (21% O2). In contrast, encapsulated BTZ in anti-CD38 NPs induced a significantly more profound killing effect in the normoxic MM cells, and overcame the hypoxia-induced resistance in MM cells (FIG. 13B).

To better mimic the tumor microenvironment, we have previously shown that a more realistic 3D tissue engineered bone marrow (3DTEBM) culture model, derived from the BM of MM patients, can recreate better the pathophysiology and drug resistance of MM which resembles tissue depth with oxygen and drug gradients, as well as, recreated the tumor microenvironment [20, 21]. BTZ (10 nM) as free drug had a very modest effect on survival of MM cells (8% killing). In contrast, encapsulated BTZ induced a significant and robust effect with a 93% killing in the 3DTEBM (FIG. 13C). We further detected the co-localization of NPs (red) and the MM cells (green) at different depths in the 3D cultures, indicating that the NP can penetrate the 3D culture and deliver BTZ (FIG. 13D-G).

### Experiment 10. Effect of the Anti-CD38 Chitosan NPs on the Efficacy in Inhibition of Tumor Progression and Reduction of the Side Effects of Bortezomib In Vivo.

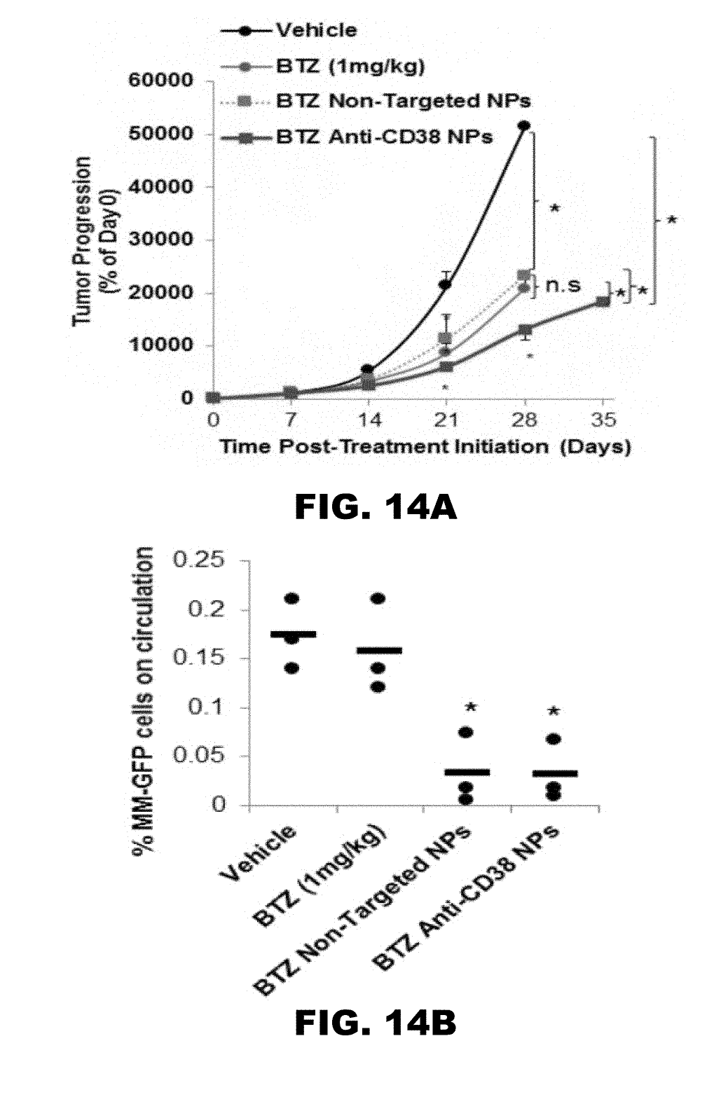

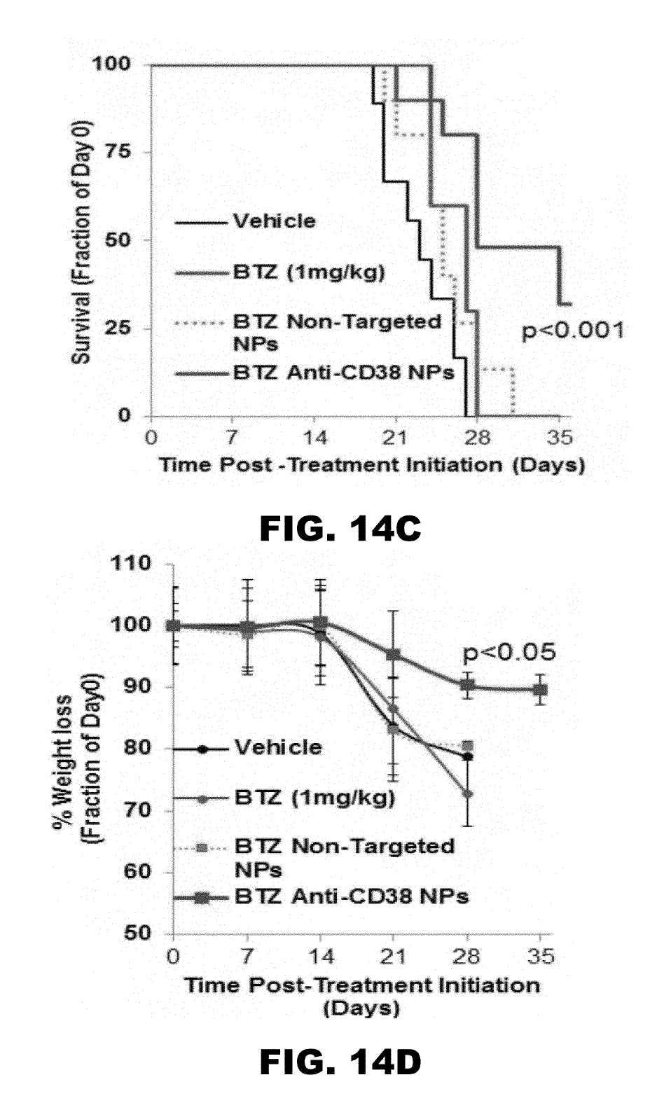

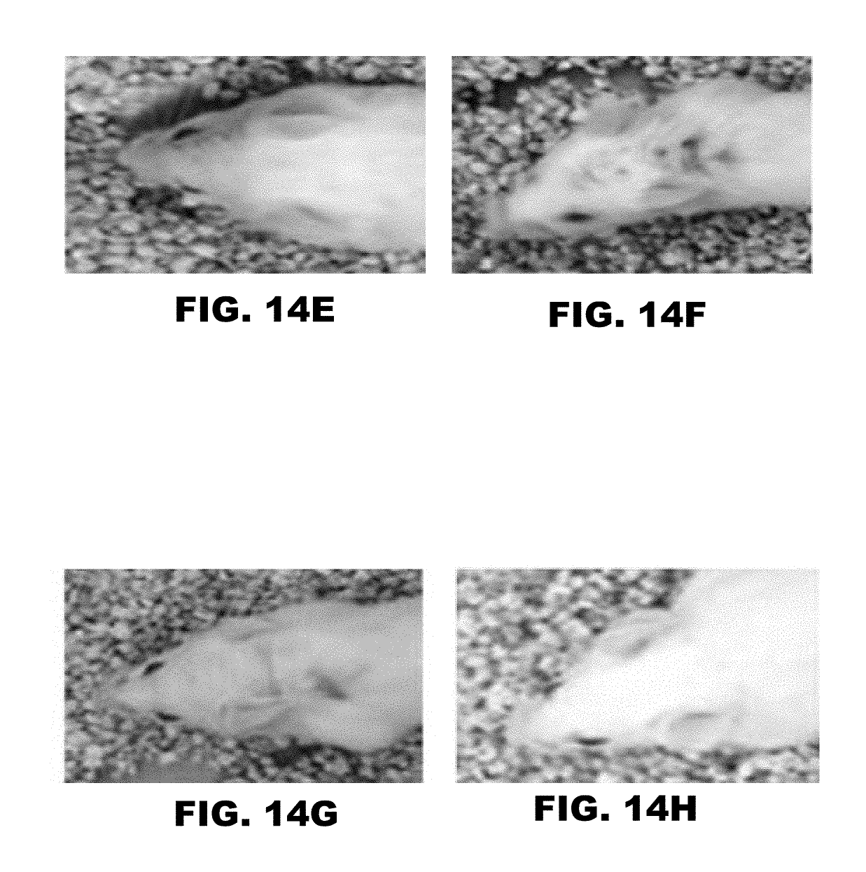

Tumor bearing mice were treated with vehicle, BTZ as a free drug (1 mg/kg once a week), non-targeted NPs loaded with BTZ (1 mg/kg once a week), and anti-CD38 NPs loaded with BTZ (1 mg/kg once a week). Tumor progression was followed in the 4 groups for 5 weeks by BLI. BTZ treatment was efficacious in reducing tumor size in all the treatment-groups (free drug, BTZ-loaded non-targeted NPs and BTZ-loaded anti-CD38 NPs) compared to vehicle treatment. However, treatment with BTZ-loaded anti-CD38 NPs reduced the tumor size to significantly lower tumor size compared to non-targeted NPs and free drug-groups. And no significant difference was observed between BTZ as free-drug and BTZ-loaded in non-targeted NPs (FIG. 14A).

In addition, the number of circulating MM cells in a blood sample taken from each group at day 25 after the beginning of the treatment revealed a significant reduction of MM cells in the BTZ-loaded NPs groups compared to free drug (FIG. 14B). Importantly, treatment with BTZ-loaded anti-CD38 NPs improved overall survival of the MM-bearing mice (32% surviving at day 35), compared to vehicle group (all the group died by day 28), the free BTZ (all the group died by day 29), and BTZ-loaded non-targeted NPs (all the group died by day 31) (FIG. 14C).

Moreover, we tested the effect of different formulations on the weight loss in the animals. Treatment anti-CD38 NPs resulted in 10% loss in body mass during the 5 weeks treatment period, whereas the free BTZ and non-targeted NPs-groups demonstrated >20% weight loss, and they were consequently sacrificed (FIG. 14D). Moreover, in both the free BTZ and non-targeted NPs-groups a significant hair loss was observed compared to the vehicle-treated (control) group, while such hair loss was not observed in the anti-CD38 NPs group (FIG. 14E-H).

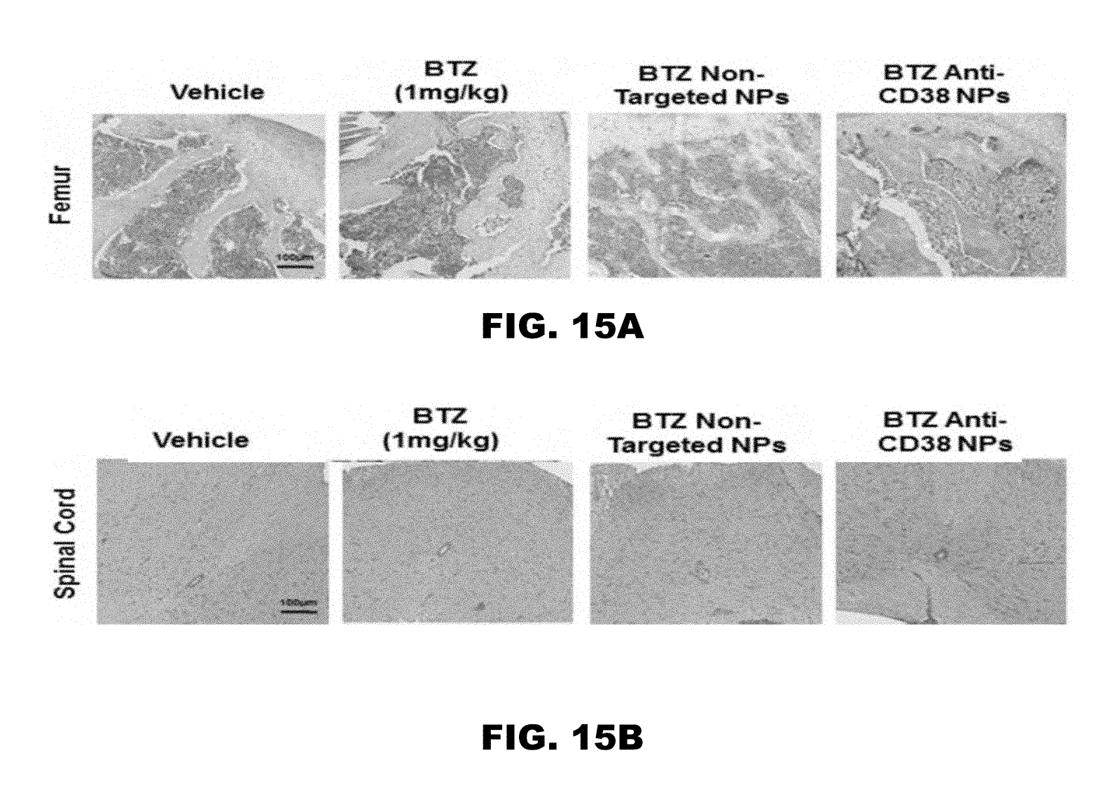

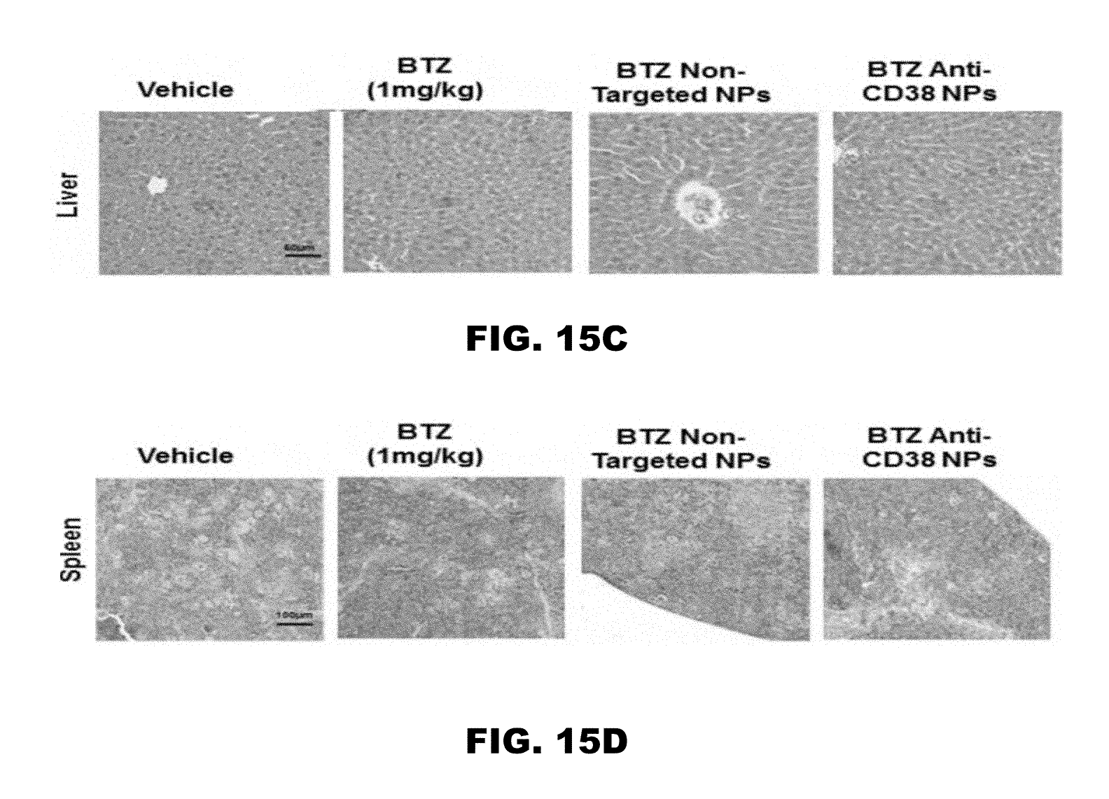

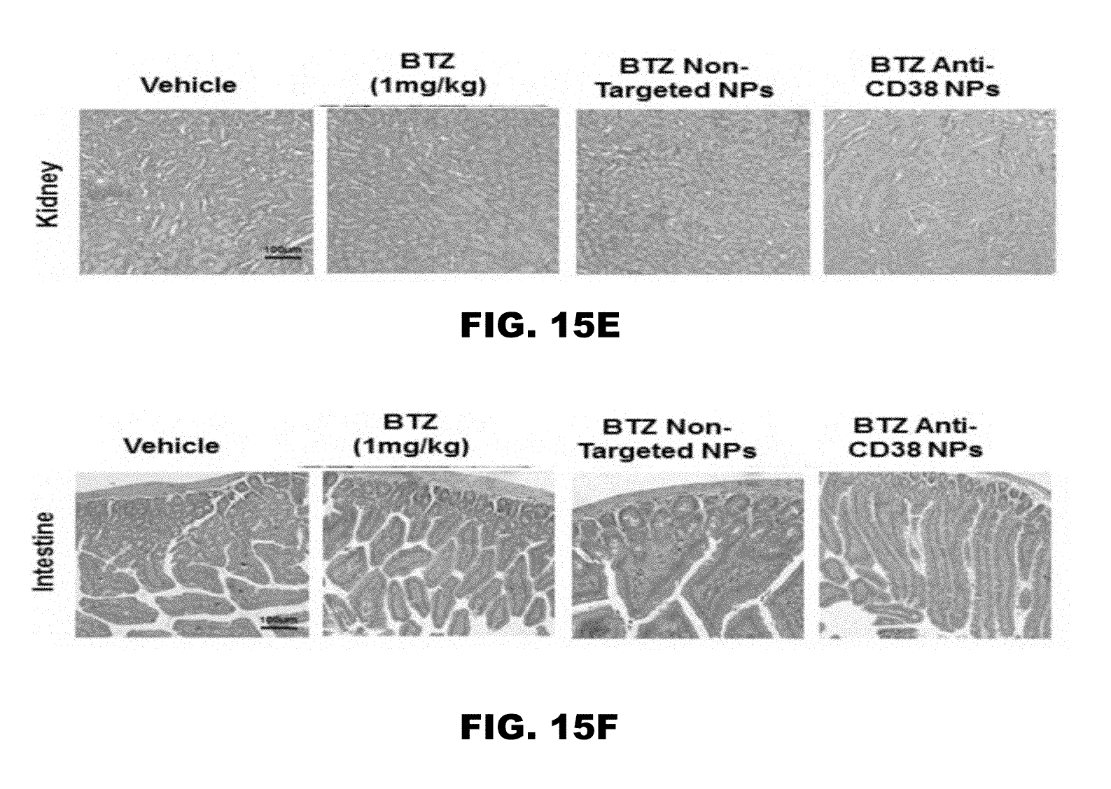

In addition, 3 mice were taken from each group at day 25 post treatment, and specimens of the BM, spleen, liver, brain, spinal-cord and intestine were fixed and pathologically evaluated for histological tissue damage. No apparent histopathologic changes in the tissues, including femurs, spinal cord, liver, spleen, kidney and intestine was observed in any of the groups (FIG. 15A-F).

### Materials and Methods for Examples

### Materials and Reagents

Unless stated otherwise, all materials were purchased from Sigma (St. Louis, Mo., USA). Chitosan low molecular weight, acetic acid 99.7%, and sodium tripolyphosphate (TPP) were used for the preparation of the chitosan NPs. Alexa Fluor 633 (AF633) was purchased from Life Technologies (Carlsbad, Calif., USA). Streptavidin conjugation kit (abcam, Cambridge, Mass., USA), Mix-n-Stain Biotin labeling kit, and human anti-CD38 (Clone 240742, R&D systems, Minneapolis, Minn., USA) were used to prepared targeted anti-CD38 NPs.

### Cells

The MM cell lines MM.1S, H929, RPMI8226, and U266 were purchased from ATCC, OPM1 and MM1s-GFP-Luc were a kind gift from Dr. Irene Ghobrial (Dana-Farber Cancer Institute, Harvard Medical School, Boston, Mass.). MSP-1 cell line was developed in our lab and used as myeloma-derived stromal cell line for co-cultures [17]. Bone Marrow mononuclear cells (BM MNCs) from 5 different patient samples were purchased from Allcells (Alameda, Calif.). Peripheral blood mononuclear cells (PB MNCs) were isolated from pheresis leukopaks from the Siteman Cancer Center, Washington University in Saint Louis. 1× RBC Lysis Buffer was added to whole blood, gently vortex and incubated at room temperature, protected from light, for 10-15 minutes. Cell were washed and cultured in RPMI completed media. Primary CD138+ cells were isolated from BM aspirates of MM patients from the Siteman Cancer Center, Washington University in Saint Louis, by magnetic-bead sorting, as previously described [18]. Informed consent was obtained from all patients with an approval from the Washington University Medical School IRB committee and in accord with the Declaration of Helsinki. All cells were cultured at 37° C., 5% CO2; MM cells in RPMI-1640 media (Corning CellGro, Mediatech, Manassas, Va.) supplemented with 10% fetal bovine serum (FBS, Gibco, Life technologies, Grand island, N.Y.), 2 mmol/l of L-glutamine, 100 U/ml penicillin, and 100 μg/ml streptomycin (Corning CellGro), and stromal cells in Dulbecco's Modified Eagle's Medium (DMEM, Corning CellGro) supplemented with 20% FBS, L-glutamine, penicillin, and streptomycin.

### Preparation of Chitosan NPs

Chitosan NPs were obtained by ionic crosslinking of a 2 mg/ml chitosan solution dissolved in 2.1% acetic acid with TPP solution (0.25-1 mg/ml). Chitosan solution was added drop-wise with a 30G needle to a TPP solution and kept with constant medium stirring for 15 minutes (Ratio 5:1 in volume). After NPs stabilization, the NPs were ultracentrifuge at 40.000g for 30 minutes at 10° C. (Rotor 25.50, Avanti J-E, Beckman Coulter, Indianapolis, Ind., USA). AF633 chitosan polymer was elaborated for in vivo, confocal, and flow cytometry studies and used for preparation of AF633 chitosan NPs. Chitosan NPs were conjugated with streptavidin using streptavidin-conjugation kit, according to the manufacturer's instructions. Anti-CD38 monoclonal antibody was labeled with biotin using biotin labeling kit according to manufacturer's instructions. Then, the streptavidin-chitosan NPs were mixed with the biotin-anti-CD38 overnight at 4° C. to obtain anti-CD38 targeted chitosan NPs. Moreover, chitosan NPs from the same batch without conjugation to streptavidin and biotin-antibody were used as non-targeted NPs.

### Characterization of Size and Stability of Anti-CD38 Chitosan NPs

The effect of TPP concentration (0.25-1 mg/ml), BTZ loading (intermediate (50 μM) and high-dose (1 mM)) and storage time (over a month) on particle size and ζ-potential (the potential difference between the dispersion medium and the surface of the nanoparticle) were determined by dynamic light scattering (DLS) using a Malvern Zetasizer Nano ZS (ZEN3600) (Malvern, Herrenberg, Germany) with a 633-nm He—Ne laser operating at a 173° angle. Malvern's Dispersion Technology Software version 6.01 was used to collect and analyze the data.

### HPLC Assay for Detection of Bortezomib

Bortezomib was analyzed using high performance liquid chromatography (HPLC, Agilent 1100 series, Santa Clara, Calif.) with a reverse phase C-18 column (Agilent Zorbax Eclipse XDB C18). A 50% acetonitrile solution in water containing 0.1% trifluoroacetic acid was used as the mobile phase at a flow rate of 1 mL/min. A calibration curve was formed by plotting the area under curve (AUC) of the BTZ HPLC peak (at retention time=2 minutes, λ=270 nm) for a concentration range of 0 to 1.5 mM. A linear correlation for the curve was obtained a R2=0.9995, with a limit of detection of approximately 25 μM.

### Bortezomib Encapsulation Efficiency in Anti-CD38 Chitosan NPs

The effect of intermediate (50 μM) and high-dose (1 mM) BTZ on the encapsulation efficiency of BTZ in NPs was assessed by the aforementioned HPLC assay (1 h BTZ incubation at 4° C. in 10 mg/ml NPs solution in PBS solution or incorporated into TPP solution before crosslinking). BTZ solutions (50 μM and 1 mM) were used as controls for each study group. After ultracentrifugation, supernatants were collected from each condition and AUC values were used in the calibration curve to determine BTZ concentration. The encapsulation efficiency (EE) was calculated as follows: % EE=100−[(Concentration BTZ-NPs supernatant/Concentration BTZ Control)*100].

In Vitro Drug Release from Anti-CD38 Chitosan NPs in Normal and Tumor Environments

MM cell lines (MM.1S, H929 and RPMI) and normal mononuclear cells (PB MNCs) were cultured in RPMI media with 10% FBS for 72 hours, conditioned media samples were centrifuged and supernatants were obtained. The pH of the media samples was tested and divided into two vials. The first vial was used as is (conditioned media), while the pH in the other vial was adjusted back to the pH of media (8.4) (conditioned media-Corrected pH), with non-MM cultured used as a control. Chitosan NPs loaded with doxorubicin (model fluorescent drug) were incubated in the different media samples (non-cultured media, normal mononuclear cells, and MM-media cultured with and without adjustment of the pH) for 24 h at 37° C. Samples were then centrifuged, supernatant extracted, and analyzed by fluorescence plate reader (Exc 480 nm/Em 580 nm).

### Characterization of the Kinetics of the Binding of the Anti-CD38 to MM Cells

First, we detected the expression of CD38 in three MM cell lines (MM.1S, H929, and RPMI8226) with the monoclonal antibody APC-anti-CD38 and analyzed the expression by detection of the mean fluorescent intensity (MFI) of APC in the MM cells by flow cytometry. Then, MM cell lines were treated with the anti-CD38 chitosan NPs pre-labeled with AF633 (1 mg/ml) for increasing times (0, 30 minutes, 2, 4, 6, 16 and 24 h), then cells were washed and analyzed for the MFI of AF633 by flow cytometry (to ascertain quantitatively the total binding). In addition, the cells were imaged using FV1000 confocal microscope with an XLUMPLFLN 20XW/1.0 immersion objective lens (Olympus, Pa., USA), with excitation of 633 nm and the emission filter of 650 long pass, to determine the sub-cellular distribution of the nanoparticle. To characterize the kinetics of potential dissociation, MM cells were treated with the AF633 anti-CD38 chitosan NPs for 2 hrs, then excess particles were washed from the cells, and the cells were put back to culture and tested for the fluorescence intensity at 0, 2, 6, 12 and 24 h after the washing by flow cytometry.

### Characterization of Specificity of the Binding of the Anti-CD38 Chitosan NPs to MM and Normal Cells In Vivo

MM cell lines (n=5, MM.1S, H929, RPMI8226, and U266), primary CD138+ MM cells isolated from MM patients (n=5), normal mononuclear cells isolated from the peripheral blood of normal subjects (n=5), and normal plasma cells isolated from the BM of normal subjects (n=5) were incubated with the targeted AF633 anti-CD38 chitosan NPs or with AF633 non-targeted chitosan NPs for 2 h. In addition, non-labeled non-targeted chitosan NPs were used as control. Cells were washed and analyzed for the MFI of AF633 by flow cytometry.

To further confirm the mechanism of binding of the targeted and non-targeted particles, MM cell lines (MM1s, H929, and RPMI8226) were treated with or without free unlabeled anti-CD38 monoclonal antibody (for blocking the epitopes), and then incubated with the targeted and the non-targeted NPs for 2 h, washed, and analyzed by flow cytometry.

### Characterization of Specificity and Biodistribution of Anti-CD38 NPs In Vivo

Approval for these studies was obtained from the Ethical Committee for Animal Experiments at Washington University in St. Louis Medical School. MM1s-GFP-Luc cells were injected into 20 female, 7-week old SCID mice (Taconic Farms, Hudson, N.Y.) intravenously (i.v.) at the concentration of 2×106 cells per mouse and tumor progression was confirmed using bioluminescent imaging (BLI) at 4 weeks post cell injection. Mice were then randomized to 4 groups of 5 mice each, and treated with (a) vehicle control, (b) free deactivated AF633 (5 mg/kg), (c) non-targeted NPs with a dye content equivalent to 5 mg/kg, (d) anti-CD38 NPs loaded with a dye content equivalent to 5 mg/kg. Mice were sacrificed 24 h post-injection of each treatment and organs were harvested (femurs (BM), blood, heart, kidney, liver, lung, and spleen). MM cells (GFP+ cells) were detected by flow cytometry and analyzed for anti-CD38 chitosan NPs uptake as MFI of AF633.

### The Effect of Bortezomib-Loaded Anti-CD38 Chitosan NPs on Cell Viability of MM Cells and Normal MNCs In Vitro

MM cell lines (MM.1S, H929, and RPMI8826) and PB MNCs were cultured with vehicle (control), BTZ as a free drug (5 nM), empty NPs, non-targeted NPs loaded with BTZ equivalent amounts to 5 nM, and anti-CD38 NPs loaded with BTZ equivalent amounts to 5 nM for 48 h. Cell viability was assessed using MTT solution followed by absorbance reading at 570 nm using a spectrophotometer as previously described [19]. Briefly, the MTT solution was added to the cells 48 h after starting the treatment, and 2-4 h later the stop solution was added.

In addition, cell proliferation assay of MM.1S cells co-culture with or without MM-derived MSP-1 stromal cell line with vehicle (control), BTZ as a free drug (5 nM), empty chitosan NPs, non-targeted chitosan NPs loaded with BTZ equivalent amounts to 5 nM, and anti-CD38 chitosan NPs loaded with BTZ equivalent amounts to 5 nM for 48 h was analyzed.

Furthermore, the effect of vehicle (control), BTZ as a free drug (5 nM), empty chitosan NPs, non-targeted chitosan NPs loaded with BTZ equivalent amounts to 5 nM, and anti-CD38 chitosan NPs loaded with BTZ equivalent amounts to 5 nM on proliferation of MM cells cultured in normoxic (21% O2) or hypoxic (1% O2) conditions for 48 h was measured.

MM.1S (pre-labeled with Invitrogen cell tracers DiO (10 μg/ml) for 1 h) were cultured in relevant three-dimensional tissue engineered bone marrow (3DTEBM) cultures through crosslinking of fibrinogen as previously described [20-24]. The effect of vehicle (control), BTZ as a free drug (10 nM), empty chitosan NPs, non-targeted chitosan NPs loaded with BTZ equivalent amounts to 10 nM, and anti-CD38 chitosan NPs loaded with BTZ equivalent amounts to 10 nM for 48 h was analyzed by flow cytometry. Higher BTZ doses (10 nM) are used in the 3DTEBM based on the known drug resistance of the cells in 3DTEBM cultures [20]. Cell proliferation assays were performed by digestion of 3DTEBM cultures with type I collagenase (25 mg/ml for 2-3 h at 37° C.), followed by flow cytometry analysis using MACSQuant Analyzer (Miltenyi Biotec), and the data was analyzed using FlowJo program v10 (Ashland, Oreg.). Moreover, 3DTEBM cultures treated with AF633 anti-CD38 chitosan NPs loaded with BTZ equivalent amounts to 10 nM were imaged to determine NPs distribution by confocal microscopy after 24 h incubation.

### The Effect of Bortezomib Loaded Anti-CD38 Chitosan NPs on Cell Cycle of MM Cells

H929 cells (1×106 cell/ml) were cultured with vehicle (control), BTZ as a free drug (5 nM), empty chitosan NPs, non-targeted chitosan NPs loaded with BTZ equivalent amounts to 5 nM, and anti-CD38 chitosan NPs loaded with BTZ equivalent amounts to 5 nM for 48 h, and cell cycle was analyzed as previously described [25]. Briefly, cells were washed, fixed with 70% ethanol, and washed again with PBS. RNA was degraded by incubation in RNAase for 30 minutes at 37° C., and the DNA was stained with PI solution for 10 minutes, then cells were analyzed by flow cytometry.

### The Effect of Bortezomib Loaded Anti-CD38 Chitosan NPs on Apoptosis of MM Cells

H929 cells (1×106 cell/ml) were cultured with vehicle (control), BTZ as a free drug (5 nM), empty chitosan NPs, non-targeted chitosan NPs loaded with BTZ equivalent amounts to 5 nM, and anti-CD38 chitosan NPs loaded with BTZ equivalent amounts to 5 nM for 48 h, and apoptosis was analyzed as previously described [26]. Cells were washed and resuspended in 1× Annexin binding buffer, incubated with Annexin V for 15 minutes followed by staining with PI for extra 15 minutes, 1× binding buffer was added, and the cells were analyzed with flow cytometry.

### The Effect of Bortezomib Loaded Anti-CD38 Chitosan NPs on Proliferation, Cell Cycle and Apoptosis Cell Signaling in MM Cells

To test the effect of BTZ as a free drug or encapsulated in chitosan NPs on proliferation, cell cycle, and apoptosis signaling, H929 cells were treated with vehicle (control), BTZ as a free drug (5 nM), empty chitosan NPs, non-targeted chitosan NPs loaded with BTZ equivalent amounts to 5 nM, and anti-CD38 chitosan NPs loaded with BTZ equivalent amounts to 5 nM for 6 h (survival) and 12 h (cell cycle and apoptosis). Cells were washed and lysed with 1× PMSF for

15 minutes. Protein concentration in the cell lysates was normalized and 50 μg of protein was loaded per lane. Electrophoresis was performed using NuPAGE 4%-12% Bis-Tris gels (Novex, Life Technologies, Grand Island, N.Y.) and transferred to a nitrocellulose membrane using iBlot (Invitrogen, Life Technologies). Membranes were blocked with 5% non-fat milk in Tris-Buffered Saline/Tween20 (TBST) buffer and incubated with primary antibodies overnight at 4° C. for proliferation signaling with pAKT and pERK1/2; for cell cycle with pRb; and for apoptosis with cleaved Caspase-3 and cleaved PARP. α-Tubulin was used as a loading control. The membranes were washed with TBST for 30 minutes, incubated for 1 hr at room temperature with HRP-conjugated secondary antibody, washed, and developed using Novex ECL Chemiluminescent Substrate Reagent Kit (Invitrogen). Antibodies were purchased from Cell Signaling Technology (Danvers, Mass.). Phospho-Erk1/2 (Thr202/Tyr204) (D13.14.4E) XP® (rabbit mAb #4370), phospho-Akt (Ser473) (D9E) XP® (rabbit mAb #4060), pRb (Ser807/811) (rabbit mAb #9308), cleaved-Caspase-3 (Asp175) (5A1E) (rabbit mAb #9664), cleaved-PARP (Asp214) (D64E10) (rabbit mAb #5625), and a-Tubulin (11H10) (rabbit mAb #2125) were used at a dilution of 1:1000.

### The Effect of Bortezomib Loading in Anti-CD38 Chitosan NPs on Proteasome Inhibition Activity.

Cell extracts were prepared in NP-40 lysis buffer from control and treated cells (BTZ as a free drug (5 nM), empty chitosan NPs, non-targeted chitosan NPs loaded with BTZ equivalent amounts to 5 nM, and anti-CD38 NPs loaded with BTZ equivalent amounts to 5 nM) retrieved after 2 h treatment with BTZ. Proteasome activity was determined using the AMC-tagged peptide substrate in a Proteasome Activity Assay Kit (abcam, Cambridge, Mass., USA) according to the manufacturer's protocol. Fluorescence was determined in a SpectraMax i3 Multi-Mode Detection Platform (Molecular Devices, Sunnyvale, Calif., USA) and proteasome activity was expressed as the amount of proteasome which generates 1.0 nmol of AMC per minute at 37° C.

### The Effect of Bortezomib Loading in Anti-CD38 Chitosan NPs on Intracellular Delivery of Bortezomib by Detection of Boron Cellular Accumulation Using ICP-OES

MM.1S cells (40×106 cell/ml) were cultured with vehicle (control), BTZ as a free drug (100 nM), and anti-CD38 chitosan NPs loaded with BTZ equivalent amounts to 100 nM for 1.5 h. After the treatment the cells were spun down, washed, and cell pellet was digested in nitric acid. After dilution with deionized water, samples were microwave digested and then analyzed by ICP-OES for boron content.

### Investigation of the Anti-CD38 Chitosan NPs Uptake Mechanism

Endocytosis is critical to the uptake of NPs in order to allow toxic effects in cells. MM cells were plated on glass bottom 8 well chambers and allowed to adhere overnight. CellLight Early Endosomes-GFP BacMam 2.0 reagent (targeting rab5) was added to result in a final concentration of 30 particles per cells and allowed to incubate with the cells for 18-24 h. Cells were washed twice with PBS and incubated with AF633 anti-CD38 chitosan NPs for 24 h. Cells were again washed twice with PBS and allowed to stay in phenol red free medium for imaging live under culture conditions of 37° C. and 5% CO2 at specified time points using FV1000 confocal microscope. To understand the process of internalization of anti-CD38 chitosan NPs, MM cells were pre-treated with inhibitors of macropinocytosis and phagocytosis (cytochalasin D), clathrin-mediated endocytosis (chlorpromazine), caveolae-mediated endocytosis (nystatin), or late endocytosis (EGA), prior to exposure to anti-CD38 chitosan NPs. All the inhibitors were tested on survival of MM cells to determine a concentration that does not induce cell death by MTT. MM cells (1×106 cell/ml) were pre-incubated at 37° C. with no inhibitor (Ctrl), cytochalasin D 1 μM, chlorpromazine 2 μM, nystatin 0.5 μg/ml, and EGA 2.5 μM or at 4° C. as control of the active uptake for 30 min. Cells were washed with PBS and incubated with AF633 anti-CD38 chitosan NPs for 2 h. Then, cells were washed and analyzed by flow cytometry for the fluorescence intensity of AF633.

Proteasome activity inhibition was also measured in combination with inhibitors of macropinocytosis and phagocytosis (cytochalasin D), clathrin-mediated endocytosis (chlorpromazine), caveolae-mediated endocytosis (nystatin), or late endocytosis (EGA), prior to exposure to anti-CD38 chitosan NPs loaded with BTZ equivalent amounts to 5 nM. MM cells were pre-incubated at 37° C. with no inhibitor (Ctrl), cytochalasin D 1 μM, chlorpromazine 2 μM, nystatin 0.5 μg/ml, and EGA 2.5 μM for 30 min. Cells were washed with PBS and incubated with anti-CD38 chitosan NPs loaded with BTZ equivalent amounts to 5 nM, then proteasome Activity Assay Kit was followed as in the previous experiment.

### The Effect of Bortezomib-Loaded Anti-CD38 NPs on MM Tumor Progression In Vivo.

MM1s-GFP-Luc cells were injected into forty female, 7-week old SCID mice i.v. at the concentration of 2×106 cells per mouse and tumor progression was evaluated using BLI. After 4 weeks, tumor bearing mice were randomized into 4 groups of 10 animals each and treated with: (i) vehicle, (ii) BTZ as a free drug (1 mg/kg once a week), (iii) non-targeted NPs loaded with BTZ equivalent amounts to 1 mg/kg (once a week), (iv) and anti-CD38 NPs loaded with BTZ equivalent amounts to 1 mg/kg (once a week). Tumor progression was followed in the 4 groups twice a week (for 5 weeks) by BLI; moreover the survival, weight, and general health of the animals was followed. In addition, 3 mice were taken from each group 3 weeks after the beginning of the treatment, and specimens of the BM, blood smear, spleen, liver, kidney, brain, spinal-cord and intestine were fixed and pathologically evaluated for treatment efficacy and histological tissue damage.

### Discussion for Examples 1-10

The establishment of more effective treatments that can circumvent chemoresistance in MM is a priority. BTZ, the first proteasome inhibitor approved by the FDA for the treatment of MM, remains one of the most potent proteasome inhibitors available [32]. MM cells exhibit a greater sensitivity to proteasome activity inhibition compared to healthy cells, resulting in an accumulation of pro-apoptotic proteins, cyclins, and cyclin-dependent kinase inhibitors, while decreasing NF-KB activity within tumor cells, which ultimately results in cell cycle arrest and apoptosis [33, 34]. However, despite its success in the clinic, BTZ still possesses limitations related to dose limiting side effects [35, 36]. Therefore, novel approaches that reduce the systemic toxicity of BTZ while enhancing or improving its anti-tumor efficacy is of utmost importance.

In this study, we developed NPs-mediated targeted delivery of BTZ to MM cells specifically to improve the therapeutic index relative to free drug; though increased efficacy and specificity to MM cells and reducing the side effects in normal tissue; by targeting the NPs to CD38 which is overexpressed on MM cells.

NPs drug delivery systems allow the delivery of larger doses of chemotherapies and increase the drug bioavailability into targeted areas, thus sparing healthy tissues [13]. Chitosan has been widely used in drug delivery systems. NPs synthesized from chitosan have gained prominence due to their large drug loading capacity, superior adsorption capabilities, and long shelf life. Chitosan also possesses an abundance of hydroxyl and amino functional groups, allowing for NPs to be synthesized by physical and/or chemical crosslinking [37]. Instead of using harsh conditions in the formulation of chitosan NPs, our method is determined by ionotropic gelation, which simply involves the interaction of an ionic polymer with oppositely charge ion to initiate crosslinking [38]. TPP is a polyanion, which interacts with the cationic chitosan by electrostatic forces [39]. These chitosan NPs were further rationally targeted to MM cells by streptavidin-biotin linkage to anti-CD38 antibody (FIG. 1A-G).

We have fine-tuned the production of anti-CD38 chitosan NPs with a small size (50 nm) to allow better penetration to tumors [14, 15, 40], and we showed that these NPs were stable at different storage conditions and periods (FIG. 1A-G).

The chitosan NPs showed preferential drug release in MM tumor microenvironment compared to normal tissue microenvironment, and this release was pH dependent (FIG. 3A-E). We have previously shown that chitosan swelling (as a first step for drug release) is pH dependent, in which chitosan swelling is increased in acidic pH and enhance release for its encapsulated materials [41], due to hydration of the protonated amine groups under acidic conditions [42]. Tumor microenvironment is reported to be more acidic than normal healthy tissue [27-29], and our model demonstrated similar pH in the MM cultures to these reported in the literature This explains our findings that the chitosan NPs in this study had preferential release in the acidic tumor micro environment in MM compared with normal microenvironment. This is an interesting phenomenon which demonstrates another aspect of specificity of the NPs to tumor environment.

Several strategies have been developed to improve the delivery of chemotherapies to MM by targeting different moieties expressed on MM cells, including: (1) Very Late Antigen-4 (VLA-4), an integrin receptor expressed on cancer of hematopoietic origin such as MM, has led to very promising results with several different drug combinations [43-45]. However, the limitation with targeting VLA4-4 stems from its heterogeneous expression on myeloma cells, with even negligible expression in some MM cells, as well as its high expression in many other normal cells [43, 45]. (2) ATP-binding cassette (ABC) drug transporters, such as ABCG2 (breast cancer resistance protein) was used to target MM cancer stem cells and deliver placitaxel. These NPs inhibited tumor growth, increased survival by inducing apoptotic pathways, and showed less toxic side effects in comparison with the placitaxel treatment [46, 47]. However, the expression of the ABC in cancer cells is variable [48]. (3) Targeting of the bone marrow microenvironment by using alendronate PLGA-PEG NPs, which did not target the MM cell but the general bone microenvironment. This approach failed to showed specificity to MM, in which non-targeted and targeted NPs showed the same efficacy in vivo [49].

CD38 is a cell surface marker with low expression on various hematopoietic cells, but it was shown to be highly expressed on malignant MM cells [50]. Due to its high expression on MM cells, it is used as a marker for identification of MM cells [51-53], and there are several indications supporting the notion that CD38 plays significant roles in the progression of MM [50, 54]. Moreover, CD38 has been used as a therapeutic target in MM; anti-CD38 monoclonal antibodies are showing promising results of selective and efficient treatment of MM in preclinical studies and in early clinical trials [55-59]. Moreover, we have recently shown that while the expression of several plasma cell markers such as CD138, CD56, and CD20 changed in different stages of the disease, CD38 is constantly expressed on all forms of MM cells including differentiated MM cells in progressive MM models, as well as on stem cell-like MM cells in minimal residual disease models [60]. We are the first to propose the use of CD38 as a targeting moiety for a nanoparticle drug delivery system.

The in vitro binding studies demonstrated that CD38 expression was high throughout different MM cells lines and primary MM cells, and that the anti-CD38 chitosan NPs were effectively uptaken by all MM cells, with correlation to the expression of CD38, with minimal dissociation after binding (FIG. 5A-H). The uptake of the anti-CD38 chitosan NPs to MM cells (cell lines and primary) was significantly higher than their uptake in normal cells (4-fold of BM MNCs, and 10-fold of PB MNC), and higher than the binding of the non-targeted NPs (3-fold in vitro, 4.5-fold in bone marrow, and 1.5 to 3-fold in other organs), demonstrating that the anti-CD38 chitosan NPs are selective and specific to MM (FIG. 7A-E).

The in vitro cellular studies with BTZ-loaded anti-CD38 chitosan NPs demonstrated enhanced cytotoxicity and apoptosis on MM cells compared to similar concentration of BTZ as free drug; demonstrated by induction of more profound cell cycle arrest, and apoptosis leading to cell death through inactivation of proliferative cell signaling (MAPK, PI3K pathways, cell cycle) and activation of pro-apoptotic pathways (PARP and Caspase-3) (FIG. 9A-D).

To further investigate the mechanism of the increased efficacy of the BTZ-loaded anti-CD38 chitosan NPs, we evaluated the effect of BTZ-loaded NPs compared to free drug in proteasome activity inhibition. We found that BTZ-loaded NPs were significantly more potent in the proteasome activity inhibition than free BTZ. To study this phenomenon, we evaluated first the accumulation of BTZ in MM cells by testing their elemental boron content (since BTZ includes a boron atom) and found that boron content in MM cells treated with BTZ-loaded anti-CD38 NPs was 3-fold higher than MM cells treated with BTZ as free drug (FIG. 10A-B). This explains the results of higher proteasome inhibition, and consequently the higher cell death induced by the BTZ loaded NPs.

In order to explain the higher bortezomib content of the cells when treated with BTZ-loaded NPs, we investigated the mechanism of uptake of the anti-CD38 NPs. We hypothesized that endocytosis is the preferential internalization route for the anti-CD38 NPs. Endocytosis is a general term for the internalization of different components by the invagination of the plasma membrane and the formation of vesicles and vacuoles through membrane fission [61]. To confirm that the process involved in the internalization of the anti-CD38 chitosan NPs was endocytosis, we have shown that the NPs distribution in the cells co-localized with the distribution of endosomes (detected by rab5). Moreover, endocytosis is an active energy-dependent process [62, 63], and stopping the energy production in the cells by pre-incubation at 4° C. induced significant 50% reduction of the anti-CD38 NPs uptake, again as a confirmation that it is endocytosis-dependent (FIG. 12A-I). These results are in agreement with previously reported in the literature, demonstrating the active uptake of other types of NPs through endocytosis [64-66].

To further investigate the specific routes of uptake by endocytosis, we tested the contribution of macropinocytosis, early endocytosis pathways (clathrin or caveolae-mediated), and late endocytosis internalization. We found that macropinocytosis did not play a role in the uptake of anti-CD38 NPs. Rather, we found that the anti-CD38 NPs were uptaken through clathrin-mediated and caveolae-mediated early endocytosis which progressed to late endocytosis and delivery of the NPs content into the cells (FIG. 12G). It has been previously characterized that after CD38 ligation to mAbs internalization through endocytosis pathways takes place, where specifically CD38 intracellular movements go from early endosomes to late endosomes [67]. Since the anti-CD38 chitosan NPs tested in our work have a specific surface functionalization, it is conceivable that they entered cells by a receptor-mediated endocytic pathway.

Finally, we linked the internalization through endocytic pathways of anti-CD38 NPs to the enhanced proteasome activity inhibition of these NPs in MM cells. Treatment of MM cells with clathrin and caveolae-mediated early endocytosis, or late endocytosis inhibitors (but not macropinocytosis inhibitors) significantly reduced the proteasome inhibition of BTZ-loaded anti-CD38 chitosan NPs (FIG. 12H). When BTZ is administered as a free drug, it penetrated through passive diffusion into MM cells as well as in normal cells, and inhibition of the different endocytosis routes did not induce any change in its ability to inhibit the proteasome (FIG. 12I).

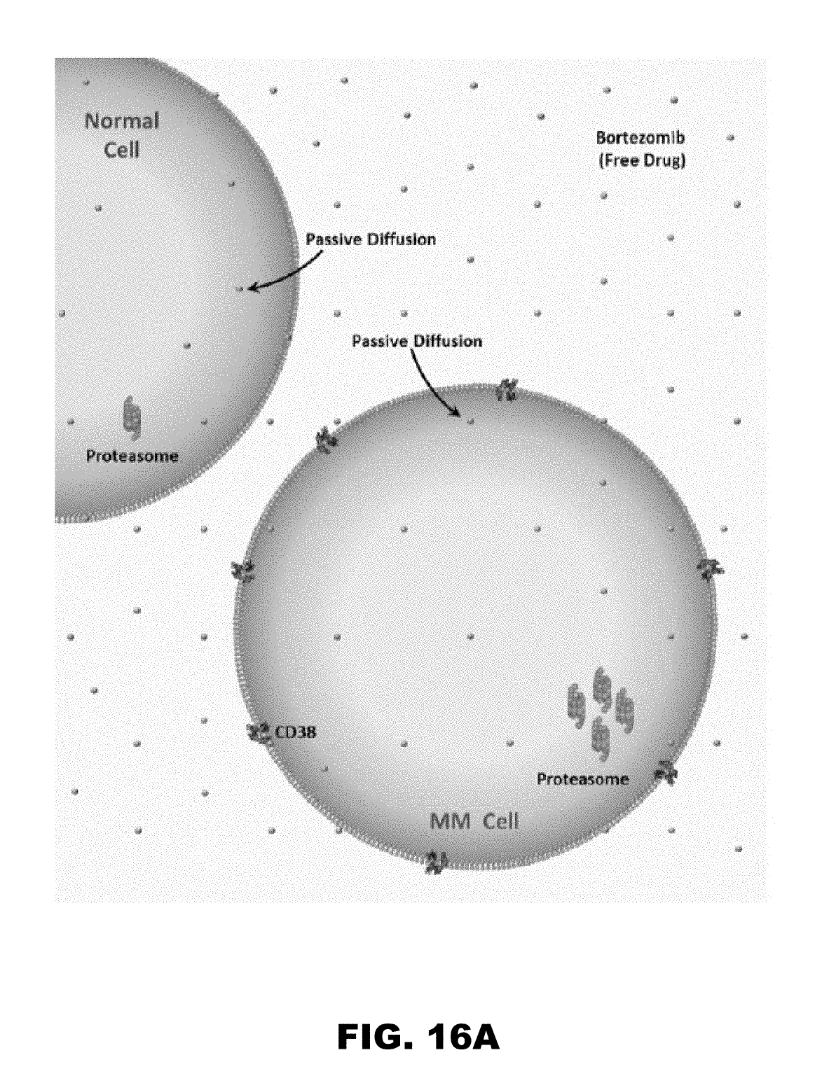

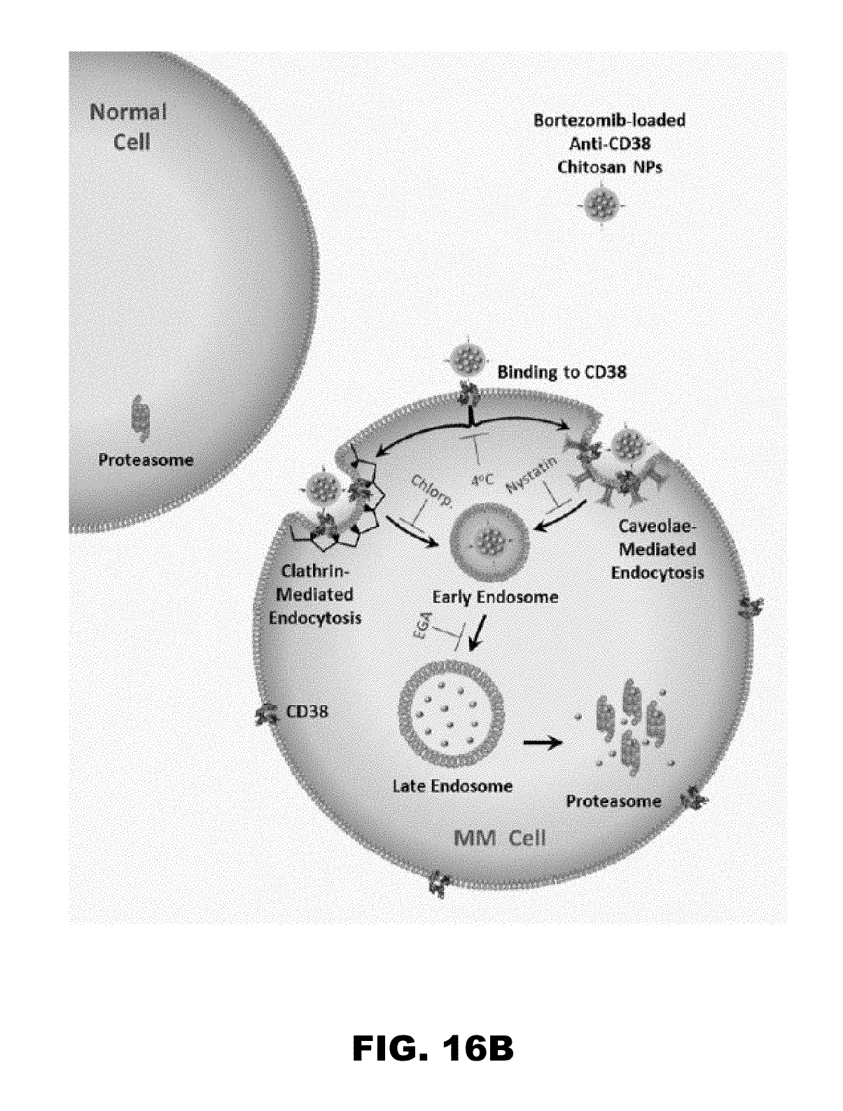

It is noteworthy that, this is the first time that internalization of a nanoparticle delivery system through receptor-mediated endocytosis is linked to the proteasome inhibition in MM, which adds another level of specificity to the effect of bortezomib compared to the free-penetration by passive diffusion of the free drug to both MM and normal cells. Once the NPs are internalized, they released the drug in the proteasome, making it more available and resulting in a more effective proteasome inhibition (a summary of that suggested mechanism is illustrated in the cartoon in FIG. 16A-B). The interaction between the proteasome and the endocytic pathway has been described before in the context of internalization and delivery of content of viruses such as Kaposi's Sarcoma-associated herpesvirus, influenza virus, and adeno-associated virus [68-70].

Furthermore, two recent papers have investigated the cytotoxic effect of NPs and their direct effect on proteasome activity. Phukan et al have shown that high doses (1.0 μg/μl) of silica-coated magnetic nanoparticles were reported to be cytotoxic in neurons by induction of ROS generation and proteasome activity reduction [71]. Armand et al have also shown that long-term exposure (2 months) of titanium dioxide nanoparticles to A549 epithelial alveolar cells modifies the cellular content of proteins involved in intra- and extracellular trafficking and proteasome activity [72]. However, our current study is the first to describe the impact of the interaction between endocytosis and proteasome, in the context of endocytosis-driven delivery of NPs for the enhancement of the efficacy and specificity of proteasome inhibitors.

Then, we tested the role of NPs drug delivery on drug resistance induced by the tumor microenvironment. We have previously reported that the tumor microenvironment (including CAM-DR and hypoxia) plays a crucial role in the resistance of MM cells to BTZ [53, 73, 74]. In this study, we found that BTZ-loaded anti-CD38 NPs proved to be more efficacious than free BTZ and overcame CAM-DR and hypoxia-mediated drug resistance (FIGS. 13A and 13B). In addition, 3D culture systems are gaining strength as in vitro systems to assess and predict drug sensitivity in myeloma [20, 75, 76]. Therefore, we used a relevant 3D model made from bone marrow supernatants of MM patients, which recreates the tumor microenvironment, to evaluate NPs as drug delivery systems and predict better the in vivo performance of NPs. The 3DTEBM model corroborated the increase cytotoxicity of anti-CD38 chitosan NPs on MM cells relative to free drug and allowed to corroborated co-localization of the NPs in the MM cells (FIGS. 13C and 13D). These results establish the significance of targeting MM cells as well as their interactions with the microenvironment in the design of more effective novel therapeutic approaches. However, it is important to mention that both BTZ-loaded chitosan NPs (anti-CD38 chitosan NPs and non-targeted chitosan NPs) were equally effectively in vitro increasing cytotoxicity, apoptosis, and overcoming drug resistance. This is most likely due to the long incubation periods needed for the cytotoxic effects in vitro, which eliminates the kinetic advantage of the binding of the anti-CD38 NPs over the non-targeted NPs in the in vitro stationary system. This is in the contrast to the in vivo system where the fast binding kinetics is crucial, due to elimination of the unbound particles.

Indeed, anti-CD38 NPs were further supported in the in vivo studies which demonstrated that although both NPs were equally effective in vitro, the anti-CD38 targeted NPs had a markedly improved tumor growth inhibition, improved overall survival, and reduction of side effects compared to the non-targeted NPs (FIG. 14A-H). For our in vivo study, we used the MM.1S injected model to develop MM-bearing mice with tumors mainly in the bone marrow recreating the pathophysiology of the disease including the BM microenvironment; with the treatment performed once a week at a dose 1 mg/kg BTZ intravenously similar to human clinical protocols. In vivo, the BTZ-loaded anti-CD38 NPs were more efficacious in delaying tumor progression in the BM and circulating tumor cells, improved overall survival, and reduction of systemic side effects measured by body weight loss compared to non-targeted chitosan NPs and free drug, and lower toxicity such as hair loss (FIG. 14A-H), with no histological toxicity in any the organs.

The major limitation of our studies was than although anti-CD38 chitosan NPs were able to show significant inhibition of tumor growth and improved overall survival compared to free drug, the results could be improved. It should be taken into consideration that due to the low toxicity of the BTZ-loaded anti-CD38 NPs, higher doses could be used in future experiments to achieve a better tumor growth inhibition and overall survival, as well as, initiate treatment at earlier times points to improve therapeutic outcome.

While this study focuses solely on the encapsulation of BTZ into anti-CD38 chitosan NPs, the methodology presented could also be applied to other therapeutic or diagnostic molecules, especially other proteasome inhibitors for improved efficacy and safety profile.

In conclusion, we have developed and characterized anti-CD38 chitosan NPs for the delivery of BTZ in MM showing preferential BTZ release in tumor-microenvironment, specific binding to MM cells, and an improved drug cellular uptake through endocytosis, which translated in enhanced proteasome inhibition and robust cytotoxic effect on MM cells. Furthermore, the anti-CD38 chitosan NPs specifically delivered therapeutic agents to MM cells improving therapeutic efficacy and reducing side effects in vivo. We are the first to report the enhancement of the efficacy and specificity of proteasome inhibitors due to endocytosis-driven delivery of NPs. The findings in this manuscript are the basis for a provisional patent application, an IND application, and future clinical trials to test the BTZ-loaded anti-CD38 NPs as a novel therapeutic approach in MM.

### REFERENCES

[1] R. L. Siegel, K. D. Miller, A. Jemal, Cancer statistics, 2016, CA: a cancer journal for clinicians, 66 (2016) 7-30.
[2] R. A. Kyle, S. V. Rajkumar, Multiple myeloma, The New England journal of medicine, 351(2004) 1860-1873.
[3] W. I. Gonsalves, P. Milani, D. Derudas, F. K. Buadi, The next generation of novel therapies for the management of relapsed multiple myeloma, Future oncology (London, England), 13 (2017) 63-75.
[4] W. I. Gonsalves, S. V. Rajkumar, M. A. Gertz, A. Dispenzieri, M. Q. Lacy, F. K. Buadi, D. Dingli, R. S. Go, N. Leung, P. Kapoor, S. R. Hayman, J. A. Lust, S. J. Russell, S. R. Zeldenrust, Y. L. Hwa, T. V. Kourelis, R. A. Kyle, S. K. Kumar, Clinical course and outcomes of patients with multiple myeloma who relapse after autologous stem cell therapy, Bone marrow transplantation, 51 (2016) 1156-1158.
[5] P. de la Puente, B. Muz, F. Azab, M. Luderer, A. K. Azab, Molecularly Targeted Therapies in Multiple Myeloma, Leukemia Research and Treatment, 2014 (2014) 976567.
[6] P. de la Puente, A. K. Azab, Contemporary drug therapies for multiple myeloma, Drugs of today (Barcelona, Spain: 1998), 49 (2013) 563-573.
[7] J. Adams, The proteasome: a suitable antineoplastic target, Nature reviews. Cancer, 4 (2004) 349-360.
[8] R. LeBlanc, L. P. Catley, T. Hideshima, S. Lentzsch, C. S. Mitsiades, N. Mitsiades, D. Neuberg, O. Goloubeva, C. S. Pien, J. Adams, D. Gupta, P. G. Richardson, N. C. Munshi, K. C. Anderson, Proteasome Inhibitor PS-341 Inhibits Human Myeloma Cell Growth in Vivo and Prolongs Survival in a Murine Model, Cancer Research, 62 (2002) 4996.
[9] A. Field-Smith, G. J. Morgan, F. E. Davies, Bortezomib (Velcadetrade mark) in the Treatment of Multiple Myeloma, Therapeutics and clinical risk management, 2 (2006) 271-279.
[10] B. Cao, J. Li, X. Mao, Dissecting bortezomib: development, application, adverse effects and future direction, Current pharmaceutical design, 19 (2013) 3190-3200.
[11] J. F. S. Miguel, P. Richardson, P. Sonneveld, M. Schuster, D. Irwin, E. Stadtmauer, T. Facon, J. Harousseau, D. Ben-Yehuda, S. Lonial, H. Goldschmidt, D. Reece, J. Blade, M. Boccadoro, J. Cavenagh, W. Dalton, A. Boral, D. Schenkein, K. Anderson, Frequency, Characteristics, and Reversibility of Peripheral Neuropathy (PN) in the APEX Trial, Blood, 106 (2005) 366-366.
[12] J. D. Ashley, J. F. Stefanick, V. A. Schroeder, M. A. Suckow, T. Kiziltepe, B. Bilgicer, Liposomal bortezomib nanoparticles via boronic ester prodrug formulation for improved therapeutic efficacy in vivo, Journal of medicinal chemistry, 57 (2014) 5282-5292.
[13] P. de la Puente, A. K. Azab, Nanoparticle delivery systems, general approaches and their implementation in Multiple Myeloma, European journal of haematology, (2017).
[14] H. Maeda, The enhanced permeability and retention (EPR) effect in tumor vasculature: the key role of tumor-selective macromolecular drug targeting, Advances in enzyme regulation, 41 (2001) 189-207.
[15] R. K. Jain, T. Stylianopoulos, Delivering nanomedicine to solid tumors, Nature reviews. Clinical oncology, 7 (2010) 653-664.
[16] J. Fang, H. Nakamura, H. Maeda, The EPR effect: Unique features of tumor blood vessels for drug delivery, factors involved, and limitations and augmentation of the effect, Advanced drug delivery reviews, 63 (2011) 136-151.
[17] P. de la Puente, N. Quan, R. S. Hoo, B. Muz, R. C. Gilson, M. Luderer, J. King, S. Achilefu, N. N. Salama, R. Vij, A. K. Azab, Newly established myeloma-derived stromal cell line MSP-1 supports multiple myeloma proliferation, migration, and adhesion and induces drug resistance more than normal-derived stroma, Haematologica, 101 (2016) e307-311.
[18] A. K. Azab, P. Quang, F. Azab, C. Pitsillides, B. Thompson, T. Chonghaile, J. T. Patton, P. Maiso, V. Monrose, A. Sacco, H. T. Ngo, L. M. Flores, C. P. Lin, J. L. Magnani, A. L. Kung, A. Letai, R. Carrasco, A. M. Roccaro, I. M. Ghobrial, P-selectin glycoprotein ligand regulates the interactionof multiple myeloma cells with the bone marrow microenvironment, Blood, 119 (2012) 1468-1478.
[19] P. de la Puente, B. Muz, A. Jin, F. Azab, M. Luderer, N. N. Salama, A. K. Azab, MEK inhibitor, TAK-733 reduces proliferation, affects cell cycle and apoptosis, and synergizes with other targeted therapies in multiple myeloma, Blood cancer journal, 6 (2016) e399.
[20] P. de la Puente, B. Muz, R. C. Gilson, F. Azab, M. Luderer, J. King, S. Achilefu, R. Vij, A. K. Azab, 3D tissue-engineered bone marrow as a novel model to study pathophysiology and drug resistance in multiple myeloma, Biomaterials, 73 (2015) 70-84.
[21] P. de la Puente, A. K. Azab, 3D tissue-engineered bone marrow: what does this mean for the treatment of multiple myeloma?, Future oncology (London, England), 12 (2016) 1545-1547.
[22] P. de la Puente, D. Ludena, Cell culture in autologous fibrin scaffolds for applications in tissue engineering, Experimental cell research, 322 (2014) 1-11.
[23] P. de la Puente, D. Ludena, A. Fernandez, J. L. Aranda, G. Varela, J. Iglesias, Autologous fibrin scaffolds cultured dermal fibroblasts and enriched with encapsulated bFGF for tissue engineering, Journal of biomedical materials research. Part A, 99 (2011) 648-654.
[24] P. de la Puente, D. Ludena, M. Lopez, J. Ramos, J. Iglesias, Differentiation within autologous fibrin scaffolds of porcine dermal cells with the mesenchymal stem cell phenotype, Experimental cell research, 319 (2013) 144-152.
[25] P. de la Puente, F. Azab, B. Muz, M. Luderer, J. Arbiser, A. K. Azab, Tris DBA palladium overcomes hypoxia-mediated drug resistance in multiple myeloma, Leukemia & lymphoma, 57 (2016) 1677-1686.
[26] P. de la Puente, E. Weisberg, B. Muz, A. Nonami, M. Luderer, R. M. Stone, J. V. Melo, J. D. Griffin, A. K. Azab, Identification of ILK as a novel therapeutic target for acute and chronic myeloid leukemia, Leukemia research, (2015).
[27] J. W. Wojtkowiak, D. Verduzco, K. J. Schramm, R. J. Gillies, Drug resistance and cellular adaptation to tumor acidic pH microenvironment, Molecular pharmaceutics, 8 (2011) 2032-2038.
[28] N. Raghunand, R. J. Gillies, pH and drug resistance in tumors, Drug resistance updates: reviews and commentaries in antimicrobial and anticancer chemotherapy, 3 (2000) 39-47.
[29] S. Y. Abdullah, M. K. Ali, M. M. Sabha, Type-B lactic acidosis associated with progressive multiple myeloma, Saudi medical journal, 36 (2015) 239-242.
[30] E. J. Gillespie, C. L. Ho, K. Balaji, D. L. Clemens, G. Deng, Y. E. Wang, H. J. Elsaesser, B. Tamilselvam, A. Gargi, S. D. Dixon, B. France, B. T. Chamberlain, S. R. Blanke, G. Cheng, J. C. de la Torre, D. G. Brooks, M. E. Jung, J. Colicelli, R. Damoiseaux, K. A. Bradley, Selective inhibitor of endosomal trafficking pathways exploited by multiple toxins and viruses, Proceedings of the National Academy of Sciences of the United States of America, 110 (2013) E4904-4912.
[31] D. Dutta, J. G. Donaldson, Search for inhibitors of endocytosis: Intended specificity and unintended consequences, Cellular Logistics, 2 (2012) 203-208.
[32] L. R. Dick, P. E. Fleming, Building on bortezomib: second-generation proteasome inhibitors as anti-cancer therapy, Drug discovery today, 15 (2010) 243-249.
[33] N. Mitsiades, C. S. Mitsiades, V. Poulaki, D. Chauhan, G. Fanourakis, X. Gu, C. Bailey, M. Joseph, T. A. Libermann, S. P. Treon, N. C. Munshi, P. G. Richardson, T. Hideshima, K. C. Anderson, Molecular sequelae of proteasome inhibition in human multiple myeloma cells, Proceedings of the National Academy of Sciences of the United States of America, 99 (2002) 14374-14379.
[34] T. Hideshima, C. Mitsiades, M. Akiyama, T. Hayashi, D. Chauhan, P. Richardson, R. Schlossman, K. Podar, N. C. Munshi, N. Mitsiades, K. C. Anderson, Molecular mechanisms mediating antimyeloma activity of proteasome inhibitor PS-341, Blood, 101 (2003) 1530-1534.
[35] M. P. Curran, K. McKeage, Bortezomib: a review of its use in patients with multiple myeloma, Drugs, 69 (2009) 859-888.
[36] S. Arastu-Kapur, J. L. Anderl, M. Kraus, F. Parlati, K. D. Shenk, S. J. Lee, T. Muchamuel, M. K. Bennett, C. Driessen, A. J. Ball, C. J. Kirk, Nonproteasomal targets of the proteasome inhibitors bortezomib and carfilzomib: a link to clinical adverse events, Clinical cancer research: an official journal of the American Association for Cancer Research, 17 (2011) 2734-2743.
[37] T. Lopez-Leon, E. L. Carvalho, B. Seijo, J. L. Ortega-Vinuesa, D. Bastos-Gonzalez, Physicochemical characterization of chitosan nanoparticles: electrokinetic and stability behavior, Journal of colloid and interface science, 283 (2005) 344-351.
[38] I. A. Alsarra, Evaluation of crosslinked chitosan hydrogel beads as a carrier for prolonged delivery of diclofenac sodium: in vitro and in vivo studies, Bollettino chimico farmaceutico, 143 (2004) 359-364.
[39] Y. Kawashima, T. Handa, A. Kasai, H. Takenaka, S. Y. Lin, Y. Ando, Novel method for the preparation of controlled-release theophylline granules coated with a polyelectrolyte complex of sodium polyphosphate-chitosan, Journal of pharmaceutical sciences, 74 (1985) 264-268.
[40] V. Torchilin, Tumor delivery of macromolecular drugs based on the EPR effect, Advanced drug delivery reviews, 63 (2011) 131-135.
[41] A. K. Azab, B. Orkin, V. Doviner, A. Nissan, M. Klein, M. Srebnik, A. Rubinstein, Crosslinked chitosan implants as potential degradable devices for brachytherapy: in vitro and in vivo analysis, Journal of controlled release: official journal of the Controlled Release Society, 111 (2006) 281-289.
[42] D. R. Bhumkar, V. B. Pokharkar, Studies on effect of pH on cross-linking of chitosan with sodium tripolyphosphate: a technical note, AAPS PharmSciTech, 7 (2006) E50.
[43] T. Kiziltepe, J. D. Ashley, J. F. Stefanick, Y. M. Qi, N. J. Alves, M. W. Handlogten, M. A. Suckow, R. M. Navari, B. Bilgicer, Rationally engineered nanoparticles target multiple myeloma cells, overcome cell-adhesion-mediated drug resistance, and show enhanced efficacy in vivo, Blood cancer journal, 2 (2012) e64.
[44] J. D. Ashley, J. F. Stefanick, V. A. Schroeder, M. A. Suckow, N. J. Alves, R. Suzuki, S. Kikuchi, T. Hideshima, K. C. Anderson, T. Kiziltepe, B. Bilgicer, Liposomal carfilzomib nanoparticles effectively target multiple myeloma cells and demonstrate enhanced efficacy in vivo, J Control Release, 196 (2014) 113-121.
[45] D. Soodgupta, D. Pan, G. Cui, A. Senpan, X. Yang, L. Lu, K. N. Weilbaecher, E. V. Prochownik, G. M. Lanza, M. H. Tomasson, Small Molecule MYC Inhibitor Conjugated to Integrin-Targeted Nanoparticles Extends Survival in a Mouse Model of Disseminated Multiple Myeloma, Mol Cancer Ther, 14 (2015) 1286-1294.
[46] C. Yang, F. Xiong, J. Wang, J. Dou, J. Chen, D. Chen, Y. Zhang, S. Luo, N. Gu, Anti-ABCG2 monoclonal antibody in combination with paclitaxel nanoparticles against cancer stem-like cell activity in multiple myeloma, Nanomedicine (Lond), 9 (2014) 45-60.
[47] C. Yang, X. He, L. Song, X. Zhan, Y. Zhang, J. Dou, N. Gu, Gamma-Fe2O3 nanoparticles increase therapeutic efficacy of combination with paclitaxel and anti-ABCG2 monoclonal antibody on multiple myeloma cancer stem cells in mouse model, Journal of biomedical nanotechnology, 10 (2014) 336-344.
[48] G. Szakacs, J. P. Annereau, S. Lababidi, U. Shankavaram, A. Arciello, K. J. Bussey, W. Reinhold, Y. Guo, G. D. Kruh, M. Reimers, J. N. Weinstein, M. M. Gottesman, Predicting drug sensitivity and resistance: profiling ABC transporter genes in cancer cells, Cancer cell, 6 (2004) 129-137.
[49] A. Swami, M. R. Reagan, P. Basto, Y. Mishima, N. Kamaly, S. Glavey, S. Zhang, M. Moschetta, D. Seevaratnam, Y. Zhang, J. Liu, M. Memarzadeh, J. Wu, S. Manier, J. Shi, N. Bertrand, Z. N. Lu, K. Nagano, R. Baron, A. Sacco, A. M. Roccaro, O. C. Farokhzad, I. M. Ghobrial, Engineered nanomedicine for myeloma and bone microenvironment targeting, Proceedings of the National Academy of Sciences of the United States of America, 111 (2014) 10287-10292.
[50] A. Chillemi, G. Zaccarello, V. Quarona, M. Lazzaretti, E. Martella, N. Giuliani, R. Ferracini, V. Pistoia, A. L. Horenstein, F. Malavasi, CD38 and bone marrow microenvironment, Front Biosci (Landmark Ed), 19 (2014) 152-162.
[51] A. C. Rawstron, A. Orfao, M. Beksac, L. Bezdickova, R. A. Brooimans, H. Bumbea, K. Dalva, G. Fuhler, J. Gratama, D. Hose, L. Kovarova, M. Lioznov, G. Mateo, R. Morilla, A. K. Mylin, P. Omede, C. Pellat-Deceunynck, M. Perez Andres, M. Petrucci, M. Ruggeri, G. Rymkiewicz, A. Schmitz, M. Schreder, C. Seynaeve, M. Spacek, R. M. de Tute, E. Van Valckenborgh, N. Weston-Bell, R. G. Owen, J. F. San Miguel, P. Sonneveld, H. E. Johnsen, N. European Myeloma, Report of the European Myeloma Network on multiparametric flow cytometry in multiple myeloma and related disorders, Haematologica, 93 (2008) 431-438.
[52] J. F. San Miguel, N. C. Gutierrez, G. Mateo, A. Orfao, Conventional diagnostics in multiple myeloma, European journal of cancer (Oxford, England: 1990), 42 (2006) 1510-1519.
[53] B. Muz, P. de la Puente, F. Azab, M. J. Luderer, J. King, R. Vij, A. K. Azab, A CD138-independent strategy to detect minimal residual disease and circulating tumour cells in multiple myeloma, British journal of haematology, 173 (2016) 70-81.
[54] F. Malavasi, S. Deaglio, A. Funaro, E. Ferrero, A. L. Horenstein, E. Ortolan, T. Vaisitti, S. Aydin, Evolution and function of the ADP ribosyl cyclase/CD38 gene family in physiology and pathology, Physiological reviews, 88 (2008) 841-886.
[55] A. Flemming, Deal watch: J&J and Genmab deal to push forward CD38 as a blood cancer target, Nature reviews. Drug discovery, 11 (2012) 822.
[56] M. de Weers, Y. T. Tai, M. S. van der Veer, J. M. Bakker, T. Vink, D. C. Jacobs, L. A. Oomen, M. Peipp, T. Valerius, J. W. Slootstra, T. Mutis, W. K. Bleeker, K. C. Anderson, H. M. Lokhorst, J. G. van de Winkel, P. W. Parren, Daratumumab, a novel therapeutic human CD38 monoclonal antibody, induces killing of multiple myeloma and other hematological tumors, Journal of immunology (Baltimore, Md.: 1950), 186 (2011) 1840-1848.
[57] M. S. van der Veer, M. de Weers, B. van Kessel, J. M. Bakker, S. Wittebol, P. W. Parren, H. M. Lokhorst, T. Mutis, Towards effective immunotherapy of myeloma: enhanced elimination of myeloma cells by combination of lenalidomide with the human CD38 monoclonal antibody daratumumab, Haematologica, 96 (2011) 284-290.
[58] D. J. Green, N. N. Orgun, J. C. Jones, M. D. Hylarides, J. M. Pagel, D. K. Hamlin, D. S. Wilbur, Y. Lin, D. R. Fisher, A. L. Kenoyer, S. L. Frayo, A. K. Gopal, J. J. Orozco, T. A. Gooley, B. L. Wood, W. I. Bensinger, O. W. Press, A preclinical model of CD38-pretargeted radioimmunotherapy for plasma cell malignancies, Cancer research, 74 (2014) 1179-1189.
[59] J. Deckert, M. C. Wetzel, L. M. Bartle, A. Skaletskaya, V. Goldmacher, F. Vallee, Q. Zhou-Liu, P. Ferrari, S. Pouzieux, C. Lahoute, C. Dumontet, A. Plesa, M. Chiron, P. Lejeune, T. Chittenden, P. U. Park, V. Blanc, SAR650984, a novel humanized CD38-targeting antibody, demonstrates potent anti-tumor activity in models of multiple myeloma and other CD38+ hematologic malignancies, Clinical cancer research: an official journal of the American Association for Cancer Research, (2014).
[60] B. Muz, P. de la Puente, F. Azab, M. Luderer, A. Azab, Hypoxia promotes stem cell-like phenotype in multiple myeloma cells, Blood cancer journal, In press (2014).
[61] J. Huotari, A. Helenius, Endosome maturation, The EMBO journal, 30 (2011) 3481-3500.
[62] L. A. Mulcahy, R. C. Pink, D. R. Carter, Routes and mechanisms of extracellular vesicle uptake, Journal of extracellular vesicles, 3 (2014).
[63] D. Dutta, J. G. Donaldson, Search for inhibitors of endocytosis: Intended specificity and unintended consequences, Cell Logist, 2 (2012) 203-208.
[64] J. Panyam, W. Z. Zhou, S. Prabha, S. K. Sahoo, V. Labhasetwar, Rapid endo-lysosomal escape of poly(DL-lactide-co-glycolide) nanoparticles: implications for drug and gene delivery, FASEB journal: official publication of the Federation of American Societies for Experimental Biology, 16 (2002) 1217-1226.
[65] J. Gilleron, W. Querbes, A. Zeigerer, A. Borodovsky, G. Marsico, U. Schubert, K. Manygoats, S. Seifert, C. Andree, M. Stoter, H. Epstein-Barash, L. Zhang, V. Koteliansky, K. Fitzgerald, E. Fava, M. Bickle, Y. Kalaidzidis, A. Akinc, M. Maier, M. Zerial, Image-based analysis of lipid nanoparticle-mediated siRNA delivery, intracellular trafficking and endosomal escape, Nature biotechnology, 31 (2013) 638-646.
[66] Q. Wang, P. Liu, T. Gong, X. Sun, Y. Duan, Z. Zhang, Uptake mechanism and endosomal fate of drug-phospholipid lipid nanoparticles in subcutaneous and in situ hepatoma, Journal of biomedical nanotechnology, 10 (2014) 993-1003.
[67] A. Funaro, M. Reinis, O. Trubiani, S. Santi, R. Di Primio, F. Malavasi, CD38 functions are regulated through an internalization step, Journal of immunology (Baltimore, Md.: 1950), 160 (1998) 2238-2247.
[68] I. Widjaja, E. de Vries, D. M. Tscherne, A. Garcia-Sastre, P. J. Rottier, C. A. de Haan, Inhibition of the ubiquitin-proteasome system affects influenza A virus infection at a postfusion step, Journal of virology, 84 (2010) 9625-9631.
[69] W. Greene, W. Zhang, M. He, C. Witt, F. Ye, S. J. Gao, The ubiquitin/proteasome system mediates entry and endosomal trafficking of Kaposi's sarcoma-associated herpesvirus in endothelial cells, PLoS pathogens, 8 (2012) e1002703.
[70] A. M. Douar, K. Poulard, D. Stockholm, O. Danos, Intracellular trafficking of adenoassociated virus vectors: routing to the late endosomal compartment and proteasome degradation, Journal of virology, 75 (2001) 1824-1833.
[71] G. Phukan, T. H. Shin, J. S. Shim, M. J. Paik, J.-K. Lee, S. Choi, Y. M. Kim, S. H. Kang, H. S. Kim, Y. Kang, S. H. Lee, M. M. Mouradian, G. Lee, Silica-coated magnetic nanoparticles impair proteasome activity and increase the formation of cytoplasmic inclusion bodies in vitro, Scientific Reports, 6 (2016) 29095.
[72] L. Armand, M. Biola-Clier, L. Bobyk, V. Collin-Faure, H. Diemer, J.-M. Strub, S. Cianferani, A. Van Dorsselaer, N. Herlin-Boime, T. Rabilloud, M. Carriere, Molecular responses of alveolar epithelial A549 cells to chronic exposure to titanium dioxide nanoparticles: A proteomic view, Journal of Proteomics, 134 (2016) 163-173.
[73] A. K. Azab, J. M. Runnels, C. Pitsillides, A. S. Moreau, F. Azab, X. Leleu, X. Jia, R. Wright, B. Ospina, A. L. Carlson, C. Alt, N. Burwick, A. M. Roccaro, H. T. Ngo, M. Farag, M. R. Melhem, A. Sacco, N. C. Munshi, T. Hideshima, B. J. Rollins, K. C. Anderson, A. L. Kung, C. P. Lin, I. M. Ghobrial, CXCR4 inhibitor AMD3100 disrupts the interaction of multiple myeloma cells with the bone marrow microenvironment and enhances their sensitivity to therapy, Blood, 113 (2009) 4341-4351.
[74] B. Muz, P. de la Puente, F. Azab, M. Luderer, A. K. Azab, The role of hypoxia and exploitation of the hypoxic environment in hematologic malignancies, Molecular cancer research: MCR, 12 (2014) 1347-1354.
[75] J. Kirshner, K. J. Thulien, L. D. Martin, C. Debes Marun, T. Reiman, A. R. Belch, L. M. Pilarski, A unique three-dimensional model for evaluating the impact of therapy on multiple myeloma, Blood, 112 (2008) 2935-2945.
[76] T. Calimeri, E. Battista, F. Conforti, P. Neri, M.T. Di Martino, M. Rossi, U. Foresta, E. Piro, F. Ferrara, A. Amorosi, N. Bahlis, K.C. Anderson, N. Munshi, P. Tagliaferri, F. Causa, P. Tassone, A unique three-dimensional SCID-polymeric scaffold (SCID-synth-hu) model for in vivo expansion of human primary multiple myeloma cells, Leukemia, 25 (2011) 707-711.

1. A composition for treating multiple myeloma (MM), the composition comprising: chitosan nanoparticles; at least one MM cell targeting antibody conjugated to the surface of the nanoparticles; and at least one therapeutic agent encapsulated in the nanoparticles.

2. The composition of claim 1, wherein the chitosan nanoparticles are crosslinked nanoparticles, wherein chitosan is crosslinked with sodium tripolyphosphate (TPP).

3. The composition of claim 1, wherein the MM cell targeting antibody is an anti-CD38 antibody, wherein the chitosan nanoparticles conjugated to the anti-CD38 antibody, target MM cells expressing CD38.

4. The composition of claim 1, wherein the therapeutic agent encapsulated in the chitosan nanoparticles is one or more of a proteasome inhibitor or immunomodulatory drug.

5. (canceled)

6. The composition of claim 4, wherein the therapeutic agent is one or more of doxorubicin, bortezomib, carfilzomib, marizomib, ixazomib, and MLN9708.

7. (canceled)

8. (canceled)

9. (canceled)

10. The composition of claim 1, wherein the average diameter of the chitosan nanoparticles is at least 30 nm and up to 100 nm.

11. The composition of claim 1, wherein the average diameter of the chitosan nanoparticles is 50±11 nm.

12. The composition of claim 1, wherein the zeta potential of the chitosan nanoparticles is at least 30 mV.

13. The composition of claim 1, wherein the zeta potential of the chitosan nanoparticles is at least 30 mV and up to 53.3 mV.

14. (canceled)

15. A method of making anti-CD38 bortezomib (BTZ) loaded chitosan nanoparticles, the method comprising producing chitosan nanoparticles, encapsulating BTZ in the chitosan nanoparticles, and conjugating the chitosan nanoparticles with anti-CD38 antibody.

16. (canceled)

17. The method of claim 15, wherein the chitosan nanoparticles are produced by dissolving a 2 ml/ml chitosan solution in an about 0.25 mg/ml to 1 mg/ml TPP solution.

18. The method of claim 17, wherein the volume of chitosan solution to TPP solution is in the ratio of 5:1.

19. (canceled)

20. (canceled)

21. The method of claim 15, wherein an about 50 uM to 1 mM solution of BTZ is incubated with chitosan nanoparticles to encapsulate BTZ in the chitosan nanoparticles.

22. The method of claim 15, wherein an about 50 uM to 1 mM solution of BTZ is incorporated into the TPP solution before crosslinking chitosan with TPP to encapsulate BTZ in the chitosan nanoparticles.

23. A method of specifically delivering a therapeutic composition of anti-CD38 BTZ loaded chitosan nanoparticles to tumor cells of a subject, the method comprising of administering an effective amount of the therapeutic composition to the subject.

24. The method of claim 23, wherein the tumor cells are multiple myeloma (MM) cells.

25. (canceled)

26. The method of claim 23, wherein the chitosan nanoparticles target CD38 on MM cells.

27. The method of claim 26, wherein the chitosan nanoparticles enter the MM cells by an endocytic pathway.

28. The method of claim 26, wherein the chitosan nanoparticles enter the MM cells by macropinocytosis.

29. The method of claim 26, wherein BTZ from the anti-CD38 BTZ loaded chitosan nanoparticles is specifically released in the acidic tumor environment.
